(19) United States
(12) Patent Application Publication
(10) Pub. No.: US 2020/0333357 A1
(43) Pub. Date: Oct. 22, 2020

(54) NOVEL TFEB PATHWAY AGONISTS FOR METABOLIC DISEASES AND AGEING

(71) Applicant: The Board of Regents of the University of Texas System, Austin, TX (US)

(72) Inventors: Jinming GAO, Plano, TX (US); Michael A. WHITE, Dallas, TX (US); Chensu WANG, Dallas, TX (US); Yang LI, Dallas, TX (US); Bruce POSNER, Dallas, TX (US); Gang HUANG, Plano, TX (US)

(73) Assignee: The Board of Regents of the University of Texas System, Austin, TX (US)

(21) Appl. No.: 16/763,518

(22) PCT Filed: Nov. 13, 2018
(86) PCT No.: PCT/US2018/060674

§ 371 Date: May 12, 2020

Related U.S. Application Data
(60) Provisional application No. 62/585,333, filed on Nov. 13, 2017.

(51) Int. Cl. G01N 33/68; C08F 293/00; A61K 49/00

(52) U.S. Cl. CPC G01N 33/6872; G01N 2500/10; A61K 49/0054; C08F 293/00

(57) ABSTRACT

The present disclosure is directed to a nanotechnology-enabled screening strategy to identify small molecule TFEB agonists that shift maturation of autophagosomes to degradative autolysosomes.

### PRIORITY CLAIM

This application claims benefit of priority to U.S. Provisional Application Ser. No. 62/585,333, filed Nov. 13, 2017, the entire contents of which are hereby incorporated by reference.

### BACKGROUND

This invention was made with government support under R01CA71443 and R01CA176284 awarded by the National Institutes of Health. The government has certain rights in the invention.

### 1. FIELD OF THE DISCLOSURE

The present disclosure relates generally to the fields of chemistry, biology, drug discovery and medicine. More specifically, it relates to a nanotechnology-enabled screening strategy to identify small molecule TFEB agonists that shift maturation of autophagosomes to degradative autolysosomes.

### 2. BACKGROUND

Autophagosome-lysosome biogenesis is a major adaptive catabolic process that both generates nutrients and energy during starvation and maintains homeostasis under nutrient-rich conditions. Impairment of this process is mechanistically associated with metabolic disorders and ageing. In metabolic syndromes such as obesity^{1,2} and fatty liver disease^{2,3}, excess nutrients increase demand for degradative autophagy-lysosome machinery and challenge the adaptive response capacity. Ineffective digestion of macromolecules (lipids, proteins and glycogen) and impaired organelle turnover compromise metabolic activity at the tissue level, provoke intracellular stresses, and exacerbate collateral defects in insulin action or other metabolic pathologies. During ageing and within age-related disorders⁴⁻⁶, a steady decline in productive autophagy impairs clearance of defective organelles leading pathological accumulation of pro-apoptotic factors and reactive oxygen species. Therefore, pharmacological interventions that enhance lysosome function are emerging as a promising strategy to ameliorate metabolic symptoms and promote longevity.

The transcription factor EB (TFEB) positively modulates lipid catabolism⁷ and promotes longevity⁸. This is a consequence of direct induction of the ‘coordinated lysosomal expression and regulation’ (CLEAR) network⁹, which includes genes that control autophagy, lysosome biogenesis and lipolysis^{7,10-13}. TFEB belongs to microphthalmia-associated transcription factor (MITF)/transcriptional factor E (TFE) family (MiT) of basic helix-loop-helix leucine zipper transcriptional factors that includes MITF, TFEB, transcription factor E3 (TFE3) and transcription factor EC (TFEC)¹⁴⁻¹⁶. TFEB and TFE3 share extensively overlapping functions and regulatory mechanisms^{10,17-19}. Notably, TFEB/TFE3 overexpression in the liver is sufficient to mimic many transcriptional changes that occur during starvation^{7,20}. In Caenorhabditis elegans (C. elegans), overexpression of the TFEB homolog HLH-30 also increases lifespan, likely through induction of macroautophagy⁸. Consequently, TFEB/TFE3 agonists are of interest for potential therapeutic intervention for some metabolic disorders and/or ageing.

### SUMMARY

Described herein in certain embodiments are methods of screening for an agonist of a basic helix-loop-helix leucine zipper transcriptional factor of the microphthalmia-associated transcription factor (MITF)/transcriptional factor E (TFE) family (MiT), comprising (a) incubating a cell expressing a fluorescent-labeled autophagy-related polypeptide with an ultra-pH sensitive (UPS) nanoparticle solution for a first time period sufficient for an autophagy-associated organelle within the cell to uptake the UPS nanoparticle; (b) contacting the UPS nanoparticle-treated cell with a molecule for a second time period sufficient for the cell to uptake the molecule; (c) measuring a fluorescence signal of the fluorescent-labeled autophagy-related polypeptide; and (d) comparing the fluorescence signal with a control, wherein a decrease in fluorescence signal indicates the molecule is an agonist against a basic helix-loop-helix leucine zipper transcriptional factor of the MITF/TFE family.

In some embodiments, there are provided methods of screening for an agonist of a basic helix-loop-helix leucine zipper transcriptional factor of the microphthalmia-associated transcription factor (MITF)/transcriptional factor E (TFE) family (MiT), comprising (a) incubating a cell expressing a fluorescent-labeled LC3 polypeptide with a UPS nanoparticle solution for a first time period sufficient for an autophagosome within the cell to uptake the UPS nanoparticle; (b) contacting the UPS nanoparticle-treated cell with a molecule for a second time period sufficient for the cell to uptake the molecule; (c) measuring a fluorescence signal of the fluorescent-labeled LC3 polypeptide; and (d) comparing the fluorescence signal with a control, wherein a decrease in fluorescence signal indicates the molecule has an agonist activity against a basic helix-loop-helix leucine zipper transcriptional factor of the MITF/TFE family.

In some embodiments, there are provided methods of screening for a transcription factor EB (TFEB) agonist, comprising (a) incubating a cell expressing a fluorescent-labeled autophagy-related polypeptide with a UPS nanoparticle solution for a first time period sufficient for an autophagy-associated organelle within the cell to uptake the UPS nanoparticle; (b) contacting the UPS nanoparticle-treated cell with a molecule for a second time period sufficient for the cell to uptake the molecule; (c) measuring a fluorescence signal of the fluorescent-labeled autophagy-related polypeptide; and (d) comparing the fluorescence signal with a control, wherein a decrease in fluorescence signal indicates the molecule is a transcription factor EB (TFEB) agonist.

In some embodiments, there are provided methods of screening for a transcription factor E3 (TFE3) agonist, comprising (a) incubating a cell expressing a fluorescent-labeled autophagy-related polypeptide with a UPS nanoparticle solution for a first time period sufficient for an autophagy-associated organelle within the cell to uptake the UPS nanoparticle; (b) contacting the UPS nanoparticle-treated cell with a molecule for a second time period sufficient for the cell to uptake the molecule; (c) measuring a fluorescence signal of the fluorescent-labeled autophagy-related polypeptide; and (d) comparing the fluorescence signal with a control, wherein a decrease in fluorescence signal indicates the molecule is a transcription factor E3 (TFE3) agonist.

Also described herein in certain embodiments, there is provided a cell composition comprising (a) an engineered cell expressing a fluorescent-labeled autophagy-related polypeptide; (b) a UPS nanoparticle solution that buffers an autophagy-associated organelle within the engineered cell to a pH range of from about pH 4.4 to about pH 4.7; and (c) a molecule incubated with the engineered cell, wherein the molecule is incubated with the engineered cell to determine whether it is an agonist against a basic helix-loop-helix leucine zipper transcriptional factor of the microphthalmia-associated transcription factor (MITF)/transcriptional factor E (TFE) family (MiT) expressed in the engineered cell.

In additional embodiments, there is provided a cell composition comprising (a) an engineered cell expressing a fluorescent-labeled LC3 polypeptide; (b) a UPS nanoparticle solution that buffers an autophagosome within the engineered cell to a pH range of from about pH 4.4 to about pH 4.7; and (c) a molecule incubated with the engineered cell, wherein the molecule is incubated with the engineered cell to determine whether it is capable of an agonist activity against a basic helix-loop-helix leucine zipper transcriptional factor of the microphthalmia-associated transcription factor (MITF)/transcriptional factor E (TFE) family (MiT) expressed in the engineered cell.

Also provided are methods method of treating a metabolic disease or indication in a subject in need thereof, methods of treating diabetes or a diabetes related disease in a subject in need thereof, methods of treating metabolic related obesity in a subject in need thereof, methods of treating nonalcoholic fatty liver disease (NAFLD) in a subject in need thereof, methods of treating nonalcoholic steatohepatitis (NASH) in a subject in need thereof, methods of treating an aging-related disease or disorder in a subject in need thereof, or methods of modulating an immune response due to a pathogenic infection in a subject in need thereof, each comprising administering a therapeutically effective amount of a TFEB agonist, such as the molecules described herein.

Also provided herein in certain embodiments is a composition comprising a molecule identified by a method of any one of the methods set forth in the claims below and a block copolymer capable of forming a micelle.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C. A UPS-enabled compound screen design that allows for discovery of agents that promote maturation of autophagosomes to degradative autolysosomes. (FIG. 1A) A schematic showing the design of a quantitative high-throughput cell-based assay for agents that promote maturation of autophagosomes to degradative autolysosomes. Under normal conditions, GFP-LC3 is degraded in autolysosomes resulting in a mild decrease in total fluorescent intensity of GFP signals. UPS_{4.4}-buffered autolysosomes have a pH environment that is not optimal for hydrolase activation. In consequence, GFP-LC3 accumulates in the cytosol resulting in a detectable increase in GFP fluorescence intensity as compared to controls. Compounds that promote autophagic flux and lysosomal function overcome the buffering effect of UPS_{4.4} and transition the accumulated defective autolysosomes into degradative autolysosomes. This results in a reduction in GFP fluorescence intensity that is reproducibly detectable in a high-throughput setting. (FIG. 1B) Buffer capacity (b) of UPS_{4.4}, NH₄Cl, chloroquine (CQ) and polyethylenimine (PEI) was plotted as a function of pH. (FIG. 1C) Representative images showing the effect of UPS_{4.4}-TMR treatment and a subsequent nutrient-starvation on GFP-LC3 puncta accumulation. Scale bar, 20 μm.

FIGS. 2A-C. A high throughput screen for small-molecule agonists of TFEB. (FIG. 2A) Pie charts showing the composition of the top 30 hits from the autophagy screen (left) and the top 18 hits from the TFEB screen (right). The top 18 hits include 3 FDA-approved drugs (digoxin, proscillaridin A and digoxingenin), 11 natural product fractions and 4 synthetic small molecules (including alexidine dihydrochloride and cycloheximide). (FIG. 2B) Robust Z score plot of the top 18 chemicals in the TFEB screen that overlap with the top 30 hits from the autophagy screen. (FIG. 2C) Representative images of GFP-LC3 and GFP-TFEB HeLa cells treated with 370 nM DG, 3.3 μM AD and IKA and 50 nM bafilomycin A1 (Baf A1). GFP-LC3 HeLa cells were pretreated with UPS_{4.4} prior to a 4 hr compound exposure. Baf A1, which blocks autolysosomal degradation through inhibition of vacuolar ATPases, was used as a negative control. In GFP-TFEB HeLa cells, the same concentration of compounds was used without UPS₄₄, while Baf A1 was used as a positive control. Scale bars, 20 μm.

FIGS. 3A-E. Target-dependent activation of TFEB and inhibition of mTORC1 by DG, AD and IKA. (FIGS. 3A-D) siRNA-mediated depletion of the α1 subunit of Na⁺—K⁺-ATPase (FIG. 3A) and PTPMT1 (FIG. 3C) mimics the molecular weight shift of TFEB and inhibition of mTORC1 as seen in the immunoblots of DG-treated (FIG. 3A) and AD-treated (FIG. 3C) and nutrient-deprived cells (positive controls). Representative confocal images of GFP-TFEB HeLa cells treated with DG, siATP1A1 (FIG. 3B), AD, siPTPMT1 (FIG. 3D) and their corresponding controls. siRNA against LON peptidase N-terminal domain and ring finger 1 (LONRF1) was used as a negative control siRNA. The graphs represent the percentage of cells with GFP-TFEB translocation under these conditions (mean±s.d. for n=3 independent experiments, ****p<0.0001 by two-way ANOVA). Scale bar, 20 μm. (FIG. 2E) Compound-mediated inhibition of mTORC1 dependent on negative regulator TSC2 in p53^{-/-} and p53^{-/-,} TSC2^{-/-} mouse embryonic fibroblasts (MEFs). Endogenous TFEB in cells treated with 370.4 nM DG, 3.3 μM AD or IKA or DMSO examined by immunofluorescent staining. TFEB translocation percentage was quantified in the bar graph (mean±s.d. for n=3 independent experiments, ****p<0.0001 by two-way ANOVA). Scale bars, 20 μm.

FIGS. 4A-G. Engagement of distinct Ca²⁺ pathways by small-molecule agonists of TFEB. (FIG. 4A) Intracellular Ca²⁺ concentration measured in wild-type HeLa cells treated with 5 μM BAPTA-AM, 370 nM DG, 3.3 μM AD and IKA using Fura-2-AM, and the concentration difference between compound-treated and DMSO-treated cells was normalized to the Ca²⁺ concentration in DMSO-treated cells (n=3 independent experiments). (FIG. 4B) Confocal images of GFP-TFEB HeLa cells treated with 5 μM BAPTA-AM, 5 μM FK506, 10 μM compound C (Cmpd C) and 25 μM STO-609 together with 370 nM DG, 3.3 μM AD and IKA for 4 hr. Scale bar, 20 μm. (FIG. 4C) The graph represents the percentage of cells with GFP-TFEB translocation in FIG. 4B (mean±s.d. for n=3 independent experiments, ****p<0.0001 by two-way ANOVA). (FIG. 4D) Representative images of GFP-TFEB HeLa cells treated with DG, AD and IKA together with control siRNA (siLONRF1) treatment, siRNA-mediated inhibition of PPP3CB (siPPP3CB) or in combination with 5 μM calcineurin inhibitor FK506 for 4 hr. Scale bar, 20 μm. (FIG. 4E) The graph represents the percentage of cells with GFP-TFEB translocation in FIG. 4D (mean±s.d. for n=3 independent experiments, ****p<0.0001 by two-way ANOVA). Inset shows the immunoblot of HeLa cells treated with siLONRF1 or siPPP3CB. (FIG. 4F) Cell-permeable pyruvate shown toreverse the TFEB nuclear translocation induced by AD and IKA, but not DG. Scale bar, 20 μm. (FIG. 4G) The graph represents the percentage of cells with GFP-TFEB translocation in f (mean±s.d. for n=3 independent experiments, ****p<0.0001 by two-way ANOVA).

FIGS. 5A-D. Distinct Ca²⁺-dependence of small-molecular agonists of TFEB. (FIGS. 5A-B) GFP-TFEB HeLa cells before and after a 30-min treatment of 200 μM GPN (upper panel of FIG. 5A) or 300 nM TG (upper panel of FIG. 5B) and cells pre-treated 30 min with GPN followed by a treatment with DG, AD and IKA (lower panel). The graph represents the percentage of cells with GFP-TFEB translocation under these conditions (mean±s.d. for n=3 independent experiments, ****p<0.0001 by one-way ANOVA). Scale bar, 20 μm. (FIG. 5C) Representative images of GFP-TFEB HeLa cells treated with DG, AD and IKA together with control siRNA (siLONRF1) treatment, siRNA-mediated inhibition of MCOLN1 (siMCOLN1) or in combination with 5 μM FK506 (4 hr) and 300 nM TG (30 min pretreatment). The graph represents the percentage of cells with GFP-TFEB translocation under these conditions (mean±s.d. for n=3 independent experiments, ****p<0.0001 by two-way ANOVA). Scale bar, 20 μm. Inset shows the immunoblot of cells treated with siLONRF1 or siMCOLN1. (FIG. 5D) Schematics of proposed mechanism of actions of DG, AD and IKA. Cardiac glycosides, such as DG, promote binding of their molecular target (Na⁺—K⁺-ATPase) to IP3R. IP3R-dependent ER Ca²⁺ release then recharges lysosomal Ca²⁺ stores through an unclear mechanism, enabling lysosomal Ca²⁺ release through mucolipin-1 (MCOLN1). AD targets PTPMT1 in mitochondria to perturb mitochondrial function and induce ROS release. The lysosomal Ca²⁺ channel mucolipin-1responds to elevated ROS, which results in a lysosomal Ca²⁺ release. This activates calcineurin and likely additional unknown phosphatases, which de-phosphorylate TFEB and promote nuclear translocation. Furthermore, mTORC1 maintains inhibitory TFEB phosphorylation under nutrient-rich conditions. AD and IKA both increase cytosolic Ca²⁺ levels resulting in CaMKKβ and AMPK pathway activation, which in turn negatively regulates mTORC1 to promote TFEB activation. DG also inhibits the activity of mTORC1 through an unknown mechanism. Activation of TFEB promotes lysosomal biogenesis and autophagy and upregulates genes promoting lipid metabolism. DG-, AD- and IKA-related proteins/pathways were coded in purple, green and light blue.

FIGS. 6A-K. Small-molecule agonists of TFEB promote lipid metabolism and extend lifespan in vivo. (FIG. 6A) Bright-field images showing oil red O (ORO)-stained HepG2 cells treated with 1 mM oleic acid (OA) in combination with 370 nM DG, 3.3 μM AD and IKA. The graph was obtained by absorbance reading of ORO using plate reader (bars represent mean±s.d. *p<0.05, **p<0.01, ***p<0.001 by two-way ANOVA). Scale bar, 50 μm. (FIG. 6B) Food uptake (open symbols and right y-axis) and body weight change (solid symbols and left y-axis) of mice fed with regular diet (RD), high-fat diet with oral injection of DG solvent (HFD-oral ctrl) and HFD with DG oral injection (HFD-DG) three times a week starting from Day 35 as indicated by the arrows (bars represent mean±s.d. *p<0.05, **p<0.01, ***p<0.001, ****p<0.0001, HFD-DG compared with HFD-oral ctrl group by using two-way ANOVA). (c) Whole body composition analysis (EchoMRI) of the same mice as in FIG. 6B and SFIGS. 6C-D after 3 weeks of treatment with compounds or their corresponding controls. (FIGS. 6D-G) Total serum triglyceride, cholesterol, glucose and insulin levels in compound-treated mice or their corresponding control mice after 3 weeks of treatment with compounds or their corresponding controls. (FIG. 6H) Glucose levels at indicated time points after glucose challenge (left panels) and insulin challenge (right panels). In FIGS. 6B-H, n=3-5 mice per group, bars represent mean±s.d. *p<0.05, **p<0.01, ***p<0.001, ****p<0.0001 by two-way ANOVA. (FIG. 6I) Haematoxylin and eosin (H&E) staining, ORO staining and immunohistochemistry staining against p62 of liver sections isolated from mice after 3 weeks of treatment with or without compounds. HFD-i.v. ctrl, mice injected with empty PEG-PLA nanoparticles. Scale bars, 100 μm. (FIG. 6J) Representative images of HLH-30::GFP nuclear translocation in dal-1(dt2300); sqIs19 [hlh-30p::hlh-30::GFP] C. elegans treated with 5 μM IKA or DMSO. Insets show enlarged images from the white boxes in the main images. Yellow arrows denote nuclear localized HLH30::GFP. Scale bar, 100 μm and 10 μm (insets). (FIG. 6K) The Kaplan-Meier curves of dal-(2300);fem-1(hc17ts) mutant C. elegans treated with 5 μM IKA or DMSO (n=2 independent experiments, ****p<0.0001 by log-rank test).

SFIGS. 1A-H. A UPS-enabled autophagy assay that broadens dynamic activity window. (SFIG. TA) pH titration of solutions containing UPS_{4.4} nanoparticles using 0.4 M HCl. Chloroquine (CQ, pKa=8.3 and 10.4) and NH₄Cl, two small molecular bases, and polyethyleneimines (PEI) included for comparison. (SFIG. 1B) Number of GFP-LC3 puncta per cell counted after various time of incubation with UPS_{4.4} nanoprobes (n=20-30 cells from 3 independent experiments). (SFIG. 1C) Chemical structure of UPS_{4.4} polymers (poly(ethylene oxide)-b-poly(2-(dipentylamino)ethyl methacrylate), PEO-b-PD5A) with or without tetramethylrhodamine (TMR) conjugation. (SFIG. 1D) Immunofluorescent images of GFP-LC3 HeLa cells pretreated with UPS_{4.4} labeled with a fluorescent dye TMR (UPS_{4.4}-TMR) for 18 hours in DMEM (upper panel) or followed by EBSS treatment for 0.5 hours. LAMP1 was used as a lysosomal marker. Scale bar=20 μm. (SFIG. 1E) Time-course images showing the effect of UPS_{4.4}-TMR treatment on GFP-LC3 puncta accumulation and clearance in Dulbecco's Modified Eagle Medium (DMEM) and a subsequent nutrient-starvation in Earle's Balanced Salt Solution (EBSS) for indicated time. Scale bar=20 μm. (SFIG. 1F) Confocal fluorescent images of GFP-LC3 HeLa cells pretreated with UPS_{4.4}-TMR for 18 hours before being treated 100 nM baf A1 in DMEM or EBSS for 4 hours. Scale bar=μm. (SFIGS. 1G-H) GFP-LC3 HeLa cells were seeded on a 384-well plate in DMEM and were treated with (SFIG. 1H) or without (SFIG. 1G) UPS_{4.4} for 18 hours before they were transferred into EBSS or DMEM with 100 nM baf A1. Cells that stayed in the original DMEM with UPS_{4.4} being washed off (if applicable) were used as a control. GFP and Hoechst fluorescence were read from a plate reader. GFP/Hoechst ratio was calculated after subtraction of saline background. The -fold change of the GFP/Hoechst signal was calculated against the DMEM control group (mean±s.d. for n=3 independent experiments).

SFIGS. 2A-X. A cell-based screen for small-molecule TFEB agonists. (SFIG. 2A) A pie chart showing the composition of the chemical library. (SFIG. 2B) Schematic of the autophagy screen and the workflow of it. (SFIG. 2C) Distribution of all the screened chemicals over the GFP/Hoechst ratios after background correction. Two red lines indicate the positions of the positive control (wild-type HeLa cells) and the negative control (GFP-LC3 HeLa cells treated with UPS_{4.4} only), respectively. (SFIG. 2D) Robust Z score plot of all compounds in the autophagy screen. (SFIG. 2E) Robust Z score plot of the 80 primary hits of the autophagy screen in a triplicate confirmation assay. (SFIG. 2F) Schematic of the TFEB screen. (SFIG. 2G) Representative images from the high-content TFEB screen. Bafilomycin A1 was used as a positive control in the screen. Scale bar, 50 μm. (SFIG. 2H) Workflow of the TFEB screen and a schematic showing the overlapping compounds between the top 30 hit lists from the two screens. (SFIG. 2I) The % CV (coefficient of variation) value and the Z-factor was calculated for all the plates of the autophagy (buffered with UPS_{4.4}, left) and TFEB screen (right), and the individual and averaged results were as shown. (SFIG. 2J) Chemical structure of DG, and AD. (SFIG. 2K) The ¹H NMR spectrum of purified IKA at 500 MHz in DMSO-d6. (1) The ¹³C NMR spectrum of purified IKA at 100 MHz in DMSO-d6. (SFIG. 2M) Dose-response curves of DG, AD and IKA in GFP-TFEB HeLa cells. (SFIG. 2N) Dose-response curves of DG, AD and IKA in GFP-LC3 HeLa cells treated with UPS_{4.4} showing the clearance of LC3 puncta by these compounds. (SFIG. 2O) Autophagic flux and p62/SQSTM1 protein level changes were measured in HeLa cells treated with 370.4 nM DG, 3.3 μM AD and IKA in the presence or absence of 100 nM baf A1 for 4 hr (mean±s.d. for n=2 independent experiments). Untreated cells in DMEM and nutrient-deprived cells in EBSS were used as controls. LC3-II/GAPDH (middle) and p62/GAPDH (right) ratios were quantified from immunoblots using ImageJ. (SFIG. 2P) Western blot of wild-type HeLa cells treated with various doses of DG, AD and IKA (left panel). p62/SQSTM1 and GAPDH (glyceraldehyde-3-phosphate dehydrogenase) protein levels and the dose-response curves were quantified and simulated using ImageJ (mean±s.d. for n=2 independent experiments, right panel). (SFIG. 2Q) Quantitative polymerase chain reaction (qPCR) was used to quantify relative abundance of mRNA levels in HeLa cells treated with different doses (EC₁₀, EC₅₀, EC₉₀) of compounds. DMSO was used as a control (mean±s.d. for n=3 independent experiments, *p<0.05, ***p<0.001, ****p<0.0001). (SFIG. 2R) Western blot of wild-type HeLa cells treated with various doses of DG, AD and IKA. Cytosolic and nuclear TFEB and the corresponding loading controls were blotted. (SFIG. 2S) qPCR analysis result of MEFs treated with 370.4 nM DG, 3.3 μM AD and IKA for 4 hr (mean±s.d. for n=3 independent experiments, *p<0.05, ***p<0.001, ****p<0.0001). (SFIG. 2T) Endosomal maturation rate was measured in HeLa cells treated with DG, AD and IKA for 4 hr using 100 μg mL-1 always-ON/OFF-ON UPS₅₃ nanoprobes. (SFIG. 2U) Dose-response curves of cathepsin B activity in cells treated with various doses of DG, AD and IKA. Immunoblots of wild-type HeLa cells under DG, AD and IKA treatment (EC₉₀) with control siRNA (siLONRF1), siTFEB or siTFEB in combination with siTFE3 are shown in the upper panel of (SFIG. 2V) and (SFIG. 2W). Corresponding quantification of p62 protein is shown in the lower panels of (SFIG. 2V) and (SFIG. 2W) (mean±s.d., n=2, *p<0.05, **p<0.01, ***p<0.001, ****p<0.0001). (SFIG. 2X) A qPCR analysis was done on HeLa cells under the same treatment conditions as used in SFIG. 2V or SFIG. 2W). Significance testing between the siControl and siTFEB/siTFEB+siTFE3 groups in compound-treated cells was performed by two-way ANOVA (Tukey test, mean±s.d., n=3, *p<0.05, **p<0.01, ***p<0.001, ****p<0.0001).

SFIGS. 3A-D. DG and IKA inhibit the activity of mTORC1. (SFIG. 3A) Immunoblots of the indicated proteins in HeLa cells treated with indicated doses of DG. (SFIGS. 3B-C) Immunoblots of p53^{-/-} or p53^{-/-} and TSC2^{-/-} MEFs treated with 370.4 nM DG, proscillaridin A (PA) and DMSO using whole-cell-lysate (WCL) (SFIG. 3B) and cytosolic/nuclear lysate (SFIG. 3C). (SFIG. 3D) Immunoblots of wide-type HeLa cells treated with various doses (starting from 3.3 μM with a 3-fold dilution, and right-most lane is DMSO) of IKA. Phosphorylation of p70-S6 kinase (S6K) was used as the readout of mTORC1 activity.

SFIGS. 4A-G. Small-molecule agonists activate TFEB through different pathways. (SFIG. 4A) HeLa cells were pretreated with or without 5 μM BAPTA-AM for 1 hr, washed off and treated with DG, AD and IKA for 2 hrs before lysates were collected. (SFIG. 4B) Representative images of GFP-TFEB HeLa cells treated with 3.3 μM AD or in combination with 5 μM FK506, 10 μM CsA or both. The graph (right panel) represents the percentage of cells with GFP-TFEB translocation under these conditions (mean±s.d. for n=3 independent experiments, ****p<0.0001). Scale bar, 20 μm. (SFIG. 4C) Representative images of GFP-TFEB HeLa cells treated with DG, AD and IKA at indicated intermediate doses (ED₅₀), and in combination with 5 μM BAPTA-AM, 5 μM FK506, 10 μM cyclosporine A (CsA) or both calcineurin inhibitors. The graph (right panel) represents the percentage of cells with GFP-TFEB translocation under these conditions (mean±s.d. for n=3 independent experiments, **p<0.01, ****p<0.0001). Scale bar, 20 μm. (SFIG. 4D) Endogenous NFAT nuclear translocation in cells treated with high (ED₉) or intermediate (ED₅₀) doses of DG, AD and IKA alone or with FK506. The graph shows the percentage of NFAT translocation (mean±s.d. for n=3 independent experiments, **p<0.01, ****p<0.0001). (SFIG. 4E) Known AMPK activators AICAR and metformin were sufficient to activate TFEB. The graph represents the percentage of cells with GFP-TFEB translocation under these conditions (mean±s.d. for n=3 independent experiments, ****p<0.0001). Scale bar, 20 μm. (SFIGS. 4F-G) Immunoblots of wide-type HeLa cells treated with various doses (starting from 3.3 μM with a 3-fold dilution, and right-most lane is DMSO) of AD and IKA.

SFIGS. 5A-I. Small-molecule activators of TFEB engage different sources of Ca. (SFIG. 5A) HeLa cells were pretreated with or without N-acetyl-cysteine (NAC) for 1 hr and then treated with tert-butyl hydroperoxide (TBHP) or AD for 4 hrs. TBHP and NAC were used as positive and negative controls. A ROS-sensitive DNA dye CellROX Green was used to detect cellular ROS levels after these treatments. Endogenous TFEB localization was also shown. (SFIG. 5B) Quantification of the fluorescent intensity of CellROX Green in cells treated as in a was done by a plate reader. (SFIG. 5C) Quantification of endogenous TFEB nuclear translocation in SFIG. 5A. (SFIG. 5D) Cells were pretreated with or without 25 μM IP3R inhibitor Xestosporine C (Xesto) for 1 hr and then treated with DG, AD and IKA for indicated time. Quantification of TFEB translocation was shown in SFIG. 5E-G (graphs represent mean±s.d. **p<0.01, ***p<0.001, ****p<0.0001). (SFIG. 5H-I) RNA-interference were used to knock down inositol 145-trisphosphate receptor type 1 (IP3R1) in GFP-TFEB cells before they were treated with DG, AD and IKA. siLONRF1 was used as a negative control. TFEB translocation percentage was quantified from images as shown in SFIG. 5I (mean±s.d. for n=3 independent experiments, ****p<0.0001). Scale bar, 20 μm.

SFIGS. 6A-L. Small-molecule activators of TFEB decrease the body weight of fat mice without induce toxicity in major organs and improved acute lipid-accumulation induced by short-term starvation and chloroquine (CQ) treatment. (SFIG. 6A) The body weight (left y-axis, solid symbols) and food intake (right y-axis, open symbols) measured in normal-diet-fed mice treated with vehicle or 2.5 mg/kg DG. (SFIG. 6B) Drug release curves of AD (upper panel) and IKA (lower panel) in PBS. (SFIGS. 6C-D) Food uptake (open symbols) and body weight changes (solid symbols) of mice feed with regular diet (RD), high-fat diet with intravenous injection of empty PEG-PLA nanoparticles (HFD-i.v. ctrl) and HFD with AD (HFD-AD) or IKA (HFD-IKA) i.v. injections three times a week starting from Day 35 as indicated by the arrows (bars represent mean±s.d. *p<0.05, **p<0.01, ***p<0.001, ****p<0.0001, HFD-AD or HFD-IKA compared with HFD-i.v. ctrl group). (SFIG. 6E) Known TFEB target genes were upregulated in the liver rather than in the muscle of HFD mice treated with AD or IKA. qPCR analysis of mRNA levels of some known TFEB target genes, including known TFEB targets Tfeb, Csta and Mcoln1, and key regulators of lipid metabolism Ppargc1α, Ppar1α and FGF21 in the liver (upper panel) and muscle (lower panel) samples from mice treated with AD, IKA or their corresponding controls (HFD-i.v. ctrl). (SFIG. 6F) Cells were treated with various doses DG, AD and IKA for 4 hours, and cell viability was measured immediately or after 72 hours. Dose response curves were simulated using ImageJ. (SFIG. 6G) H&E staining of heart, spleen and kidney sections from mice treated with DG, AD, IKA and their corresponding controls. Scale bar, 100 μm. (SFIG. 6H) H&E staining of liver and heart sections as well as ORO and p62/SQSTM1 IHC staining of liver sections from mice under fed and fast conditions with orally injected DG and intraperitoneally injected CQ, individually and jointly, or their corresponding control (Saline). Scale bar, 50 μm. (SFIG. 6I) The body weight of mice after the treatment described in SFIG. 6H. (SFIGS. 6J-L) Total serum levels of triglyceride, cholesterol and glucose of mice after the treatment described in SFIG. 6G.

### DESCRIPTION OF ILLUSTRATIVE EMBODIMENTS

Disclosed herein in certain embodiments are nanotechnology-enabled high-throughput screens to identify small-molecule agonists of TFEB that promote autophagolysosomal activity. In some embodiments, the TFEB agonists shift maturation of autophagosomes to degradative autolysosomes. In some embodiments, the nanotechnology-enabled high-throughput screens identified TFEB agonists including digoxin (DG); the marine-derived natural product, ikarugamycin (IKA); and the synthetic compound, alexidine dihydrochloride (AD), which act on a mitochondrial target. In some embodiments, the TFEB agonists identified in the nanotechnology-enabled high-throughput screens (e.g., digoxin, ikarugamycin, and/or alexidine dihydrochloride) activate TFEB via three distinct Ca²⁺-dependent mechanisms. In further embodiments, formulation of these compounds in liver-tropic biodegradable, biocompatible nanoparticles confers hepatoprotection against diet-induced steatosis. In further embodiments, small-molecule TFEB activators are used for the treatment of metabolic and age-related disorders.

### I. CHEMICAL DEFINITIONS

### A. Most Common Chemical Groups

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, the formula

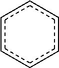

covers, for example,

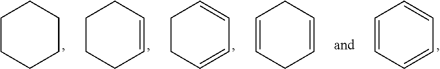

And it is understood that no one such ring atom forms part of more than one double bond. Furthermore, it is noted that the covalent bond symbol “—”, when connecting one or two stereogenic atoms, does not indicate any preferred stereochemistry. Instead, it covers all [symbol] stereoisomers as well as mixtures thereof. The symbol “ [symbol] ”, when drawn perpendicularly across a bond (e.g.,

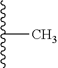

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

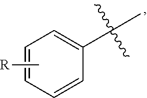

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

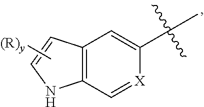

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂-(methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or

triple bonds, and no atoms other than carbon and hydrogen. The group

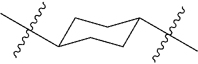 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

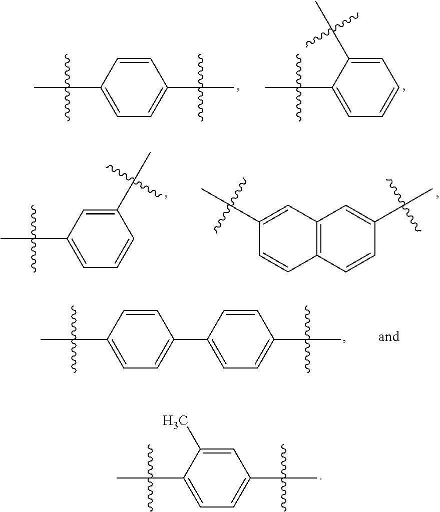

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —C₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —C₀₂H, —C₀₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —C₂H, —C₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —C₀₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier, one or more hydrogen atom has been independently replaced by —OH, —F, —Cl, —Br, —I, —NH₂, —NO₂, —C₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. When any of these terms is used with the “substituted” modifier, one or more hydrogen atom attached to a carbon atom has been independently replaced by —OH, —F, —Cl, —Br, —I, —NH₂, —NO₂, —C₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

### B. Less Common Chemical Groups

The term “heteroarenediyl” when used without the “substituted” modifier refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are be fused; however, the term heteroarenediyl does not preclude the presence of one or more alkyl or aryl groups (carbon number limitation permitting) attached to one or more ring atoms. Non-limiting examples of heteroarenediyl groups include:

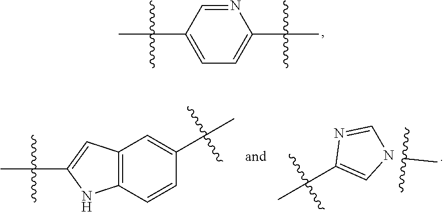

The term “heterocycloalkanediyl” when used without the “substituted” modifier refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

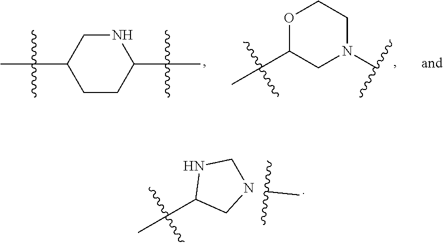

The terms “alkylsulfonyl” and “alkylsulfinyl” when used without the “substituted” modifier refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms is used with the “substituted” modifier, one or more hydrogen atom has been independently replaced by —OH, —F, —Cl, —Br, —I, —NH₂, —NO₂, —C₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylimino” when used without the “substituted” modifier refers to the divalent group ═NR, in which R is an alkyl, as that term is defined above.

The terms “phosphine” and “phosphane” are used synonymously herein. When used without the “substituted” modifier these terms refer to a compound of the formula PR₃, wherein each R is independently hydrogen, alkyl, cycloalkyl, alkenyl, aryl, or aralkyl, as those terms are defined above. Non-limiting examples include PMe₃, PPh₃, and PCy₃ (tricyclohexylphosphine). The terms “trialkylphosphine” and “trialkylphosphane” are also synonymous. Such groups are a subset of phosphine, wherein each R is an alkyl group. The term “diphosphine” when used without the “substituted” modifier refers to a compound of the formula R₂—P-L-P—R₂, wherein each R is independently hydrogen, alkyl, cycloalkyl, alkenyl, aryl, or aralkyl, and wherein L is alkanediyl, cycloalkanediyl, alkenediyl, or arenediyl. When any of these terms is used with the “substituted” modifier, one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “phosphine oxide” when used without the “substituted” modifier refers to a compound of the formula O═PR₃, wherein each R is independently hydrogen, alkyl, cycloalkyl, alkenyl, aryl, or aralkyl, as those terms are defined above. Non-limiting examples include OPMe₃ (trimethylphosphine oxide) and PPh₃O (triphenylphosphine oxide). When any of these terms is used with the “substituted” modifier, one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR′), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

### C. Common General Definitions

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylenebis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### D. Less Common Definitions

As used herein, average molecular weight refers to the weight average molecular weight (Mw) determined by static light scattering.

As used herein, a “chiral auxiliary” refers to a removable chiral group that is capable of influencing the stereoselectivity of a reaction. Persons of skill in the art are familiar with such compounds, and many are commercially available.

The term “epoxide” refers to a class of compounds of the formula:

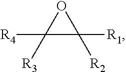

wherein R₁, R₂, and R₃ are each independently hydrogen, alkyl, and R₄ is hydrogen, alkyl, or aryl.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

“Substituent convertible to hydrogen in vivo” means any group that is convertible to a hydrogen atom by enzymological or chemical means including, but not limited to, hydrolysis and hydrogenolysis. Non-limiting examples include hydrolyzable groups, such as acyl groups, groups having an oxycarbonyl group, amino acid residues, peptide residues, o-nitrophenylsulfenyl, trimethylsilyl, tetrahydropyranyl, and diphenylphosphinyl. Non-limiting examples of acyl groups include formyl, acetyl, and trifluoroacetyl. Non-limiting examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, and β-(p-toluenesulfonyl)ethoxycarbonyl. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (omithine) and 0-Ala. Examples of suitable amino acid residues also include amino acid residues that are protected with a protecting group. Non-limiting examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), and tert-butoxycarbonyl groups (—C(O)OC(CH₃)₃). Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Non-limiting examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), and tert-butoxycarbonyl groups (—C(O)OC(CH₃)₃). Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

### II. PHARMACEUTICAL FORMULATIONS AND ROUTES OF ADMINISTRATION

For the purpose of administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound of the present disclosure formulated with one or more excipients and/or drug carriers appropriate to the indicated route of administration. In some embodiments, the compounds of the present disclosure are formulated in a manner amenable for the treatment of human and/or veterinary patients. In some embodiments, formulation comprises admixing or combining one or more of the compounds of the present disclosure with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. Pharmaceutical formulations may be subjected to conventional pharmaceutical operations, such as sterilization and/or may contain drug carriers and/or excipients such as preservatives, stabilizers, wetting agents, emulsifiers, encapsulating agents such as lipids, dendrimers, polymers, proteins such as albumin, or nucleic acids, and buffers, etc.

Pharmaceutical formulations may be administered by a variety of methods, e.g., orally or by injection (e.g. subcutaneous, intravenous, intraperitoneal, etc.). Depending on the route of administration, the compounds of the present disclosure may be coated in a material to protect the compound from the action of acids and other natural conditions which may inactivate the compound. To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds of the present disclosure may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (including, but not limited to, glycerol, propylene glycol, and liquid polyethylene glycol), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, including, but not limited to, parabens, chlorobutanol, phenol, ascorbic acid, and thimerosal. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds of the present disclosure can be administered orally, for example, with an inert diluent or an assimilable edible carrier. The compounds and other ingredients may also be enclosed in a hard or soft shell gelatin capsule, compressed into tablets, or incorporated directly into the subject's diet. For oral therapeutic administration, the compounds of the present disclosure may be incorporated with excipients and used in the form of ingestible tablets, buccal tablets, troches, capsules, elixirs, suspensions, syrups, wafers, and similar oral formulations. The percentage of the therapeutic compound in the compositions and preparations may, of course, be varied. The amount of the therapeutic compound in such pharmaceutical formulations is such that a suitable dosage will be obtained.

In some embodiments, the therapeutic compound may also be administered topically to the skin, eye, or mucosa. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In general a human equivalent dose (HED) in mg/kg can be calculated in accordance with the following formula (see, e.g., Reagan-Shaw et al., 2008, incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat.

### III. UPS NANOPARTICLES, MICELLE SYSTEMS AND COMPOSITIONS

pH is an important physiological parameter that plays a critical role in cellular and tissue homeostasis. Conventional small molecular pH sensors or buffers are limited by broad pH response. Recently, the inventors have developed a series of ultra-pH sensitive (UPS) nanoparticles based on nanoscale cooperativity as a result of supramolecular self-assembly. At pH above the apparent pKa, hydrophobic micellization dramatically sharpened the pH transitions of the block copolymers (PEO-b-PR, where PEO is poly(ethylene oxide) and PR is ionizable tertiary amine block). At pH below the pKa, micelles dissociate into cationic uimers in solution. When pH is at the pKa, the micelle nanoparticles displayed strong buffer capacity that is 70-300 folds higher than small molecular bases (e.g., chloroquine). The resulting library of micelle nanoparticles allowed an image and perturbation strategy to study organelle biology.

The systems and compositions disclosed herein utilize either a single micelle or a series of micelles tuned to different pH levels. Furthermore, the micelles have a narrow pH transition range. In some embodiments, the micelles have a pH transition range of less than about 1 pH unit. In various embodiments, the micelles have a pH transition range of less than about 0.9, less than about 0.8, less than about 0.7, less than about 0.6, less than about 0.5, less than about 0.4, less than about 0.3, less than about 0.25, or less than about 0.2. The narrow pH transition range advantageously provides a sharper pH response that can result in complete turn-on of the fluorophores with subtle changes of pH.

Accordingly, a single or series of pH-tunable, multicolored fluorescent nanoparticles having pH-induced micellization and quenching of fluorophores in the micelle core provide mechanisms for the independent control of pH transition (via polymers), fluorescence emission, or the use of fluorescence quenchers. The fluorescence wavelengths can be fine-tuned from, for example, violet to near IR emission range (400-820 nm). Their fluorescence ON/OFF activation can be achieved within no more than 0.25 pH units, which is much narrower compared to small molecular pH sensors. In some embodiments, a narrower range for fluorescence ON/OFF activation can be achieved such that the range is no more than 0.2 pH units. In some embodiments, the range is no more than 0.15 pH units. Furthermore, the use of a fluorescence quencher may also increase the fluorescence activation such that the difference between the associated and disassociated nanoparticle is greater than 50 times the associated nanoparticle. In some embodiments, the fluorescence activation is greater than 75 times higher than the associated nanoparticle This multicolored, pH tunable and activatable fluorescent nanoplatform provides a valuable tool to investigate fundamental cell physiological processes such as pH regulation in endocytic organelles, receptor cycling, and endocytic trafficking, which are related to cancer, lysosomal storage disease, and neurological disorders.

The size of the micelles will typically be in the nanometer scale (i.e., between about 1 nm and 1 μm in diameter). In some embodiments, the micelle has a size of about 10 to about 200 nm. In some embodiments, the micelle has a size of about 20 to about 100 nm. In some embodiments, the micelle has a size of about 30 to about 50 nm.

### IV. FLUORESCENCE DETECTION

Various aspects of the present disclosure relate to the direct or indirect detection of a fluorescent signal. Techniques for detecting fluorescent signals from fluorescent dyes are known to those in the art. For example, fluorescence confocal microscopy as described in the Examples below is one such technique.

Flow cytometry, for example, is another technique that can be used for detecting fluorescent signals. Flow cytometry involves the separation of cells or other particles, such as microspheres, in a liquid sample. The basic steps of flow cytometry involve the direction of a fluid sample through an apparatus such that a liquid stream passes through a sensing region. The particles should pass one at a time by the sensor and may categorized based on size, refraction, light scattering, opacity, roughness, shape, fluorescence, etc.

The measurements described herein may include image processing for analyzing one or more images of cells to determine one or more characteristics of the cells such as numerical values representing the magnitude of fluorescence emission at multiple detection wavelengths and/or at multiple time points.

### V. SPECT AND PET

Radionuclide imaging modalities (positron emission tomography, (PET); single photon emission computed tomography (SPECT)) are diagnostic cross-sectional imaging techniques that map the location and concentration of radionuclide-labeled radiotracers. Although CT and MRI provide considerable anatomic information about the location and the extent of tumors, these imaging modalities cannot adequately differentiate invasive lesions from edema, radiation necrosis, grading or gliosis. PET and SPECT can be used to localize and characterize tumors by measuring metabolic activity.

PET and SPECT provide information pertaining to information at the cellular level, such as cellular viability. In PET, a patient ingests or is injected with a slightly radioactive substance that emits positrons, which can be monitored as the substance moves through the body. In one common application, for instance, patients are given glucose with positron emitters attached, and their brains are monitored as they perform various tasks. Since the brain uses glucose as it works, a PET image shows where brain activity is high.

Closely related to PET is single-photon emission computed tomography, or SPECT. The major difference between the two is that instead of a positron-emitting substance, SPECT uses a radioactive tracer that emits low-energy photons. SPECT is valuable for diagnosing coronary artery disease, and already some 2.5 million SPECT heart studies are done in the United States each year.

PET radiopharmaceuticals for imaging are commonly labeled with positron-emitters such as ¹¹C, ¹³N, ¹⁵O, ¹⁸F, ⁸²Rb, ⁶²Cu, and ⁶⁸Ga. SPECT radiopharmaceuticals are commonly labeled with positron emitters such as ^{99m}Tc, ²⁰¹T, and ⁶⁷Ga. Regarding brain imaging, PET and SPECT radiopharmaceuticals are classified according to blood-brain-barrier permeability (BBB), cerebral perfusion and metabolism receptor-binding, and antigen-antibody binding (Saha et al., 1994). The blood-brain-barrier SPECT agents, such as ^{99m}TcO4-DTPA, ²⁰¹T1, and [⁶⁷Ga]citrate are excluded by normal brain cells, but enter into tumor cells because of altered BBB. SPECT perfusion agents such as [¹²³I]IMP, [^{99w}Tc]HMPAO, [^{99m}Tc]ECD are lipophilic agents, and therefore diffuse into the normal brain. Important receptor-binding SPECT radiopharmaceuticals include [¹²³I]QNE, [¹²³I]IBZM, and [¹²³I]iomazenil. These tracers bind to specific receptors, and are of importance in the evaluation of receptor-related diseases.

### VI. CELL COMPOSITIONS

Disclosed herein in certain embodiments, is a cell composition comprising: an engineered cell expressing a fluorescent-labeled autophagy-related polypeptide; a UPS nanoparticle solution that buffers an autophagy-associated organelle within the engineered cell to a pH range from about pH 4.4 to about pH 4.7; and a molecule incubated with the engineered cell, wherein the molecule is incubated with the engineered cell to determine whether it is an agonist against a basic helix-loop-helix leucine zipper transcriptional factor of the microphthalmia-associated transcription factor (MITF)/transcriptional factor E (TFE) family (MiT) expressed in the engineered cell.

In some embodiments, is a cell composition comprising: an engineered cell expressing a fluorescent-labeled LC3 polypeptide; a UPS nanoparticle solution that buffers an autophagosome within the engineered cell to a pH range of from about pH 4.4 to about pH 4.7; and a molecule incubated with the engineered cell, wherein the molecule is incubated with the engineered cell to determine whether it is capable of an agonist activity against a basic helix-loop-helix leucine zipper transcriptional factor of the microphthalmia-associated transcription factor (MITF)/transcriptional factor E (TFE) family (MiT) expressed in the engineered cell.

In some embodiments of the cell composition, the basic helix-loop-helix leucine zipper transcriptional factor of the MITF/TFE family is transcription factor EB (TFEB), transcription factor E3 (TFE3), transcription factor EC (TFEC), or microphthalmia-associated transcription factor (MITF). In some embodiments of the cell composition, the basic helix-loop-helix leucine zipper transcriptional factor of the MITF/TFE family is transcription factor EB (TFEB). In some embodiments of the cell composition, the basic helix-loop-helix leucine zipper transcriptional factor of the MITF/TFE family is transcription factor E3 (TFE3). In some embodiments of the cell composition, the basic helix-loop-helix leucine zipper transcriptional factor of the MITF/TFE family is transcription factor EC (TFEC). In some embodiments of the cell composition, the basic helix-loop-helix leucine zipper transcriptional factor of the MITF/TFE family is microphthalmia-associated transcription factor (MITF).

In some embodiments of the cell composition, the UPS nanoparticle solution has a buffering capacity of about pH 4.4 to about 4.7. In some embodiments of the cell composition, the UPS nanoparticle solution has a buffering capacity of about pH 4.4, about pH 4.5, about pH 4.6, or about pH 4.7. In some embodiments of the cell composition, the UPS nanoparticle solution has a buffering capacity of about pH 4.7. In some embodiments, the UPS nanoparticle solution has a buffering capacity of about pH 4.4.

In some embodiments of the cell composition, the autophagy-associated organelle comprises autophagosome, amphisome, phagophore, endosome, or lysosome. In some embodiments of the cell composition, the autophagy-associated organelle comprises autophagosome. In some embodiments, the autophagy-associated organelle comprises amphisome.

In some embodiments of the cell composition, the UPS nanoparticle solution inhibits the formation of autolysosome by the autophagosome and/or amphisome. In some embodiments of the cell composition, the UPS nanoparticle solution inhibits the formation of autolysosome by the autophagosome. In some embodiments of the cell composition, the UPS nanoparticle solution inhibits the formation of autolysosome by the amphisome.

In some embodiments of the cell composition, the molecule is a small molecule compound, a protein, a peptide, a peptidomimetic, or a polynucleotide. In some embodiments of the cell composition, the molecule is a small molecule compound. In some embodiments of the cell composition, the molecule is a protein or a peptide. In some embodiments of the cell composition, the molecule is a peptidomimetic. In some embodiments of the cell composition, the molecule is a polynucleotide.

In some embodiments of the cell composition, the fluorescent-labeled autophagy-related polypeptide comprises LC3, p62, NBR1, or NDP52. In some embodiments of the cell composition, the fluorescent-labeled autophagy-related polypeptide comprises LC3. In some embodiments of the cell composition, the fluorescent-labeled autophagy-related polypeptide comprises p62. In some embodiments of the cell composition, the fluorescent-labeled autophagy-related polypeptide comprises NBR1. In some embodiments of the cell composition, the fluorescent-labeled autophagy-related polypeptide comprises NDP52.

In some embodiments of the cell composition, the fluorescent-labeled autophagy-related polypeptide comprises a fluorescent moiety. In some embodiments of the cell composition, the fluorescent moiety comprises a fluorescent molecule or a fluorescent protein. In some embodiments of the cell composition, the fluorescent-labeled autophagy-related polypeptide comprises a fluorescent protein.

In some embodiments of the cell composition, the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyanl, Midori-Ishi Cyan, TagCFP, mTFP1, enhanced yellow fluorescent protein (EYFP), Topaz, MCitrine, YPet, TagYFP, PhiYFP, ZsYellow 1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRed1, mRaspberry, dKeima-Tandem, mPlum, or AQ143.

In some embodiments of the cell composition, the autophagy-related polypeptide is exogenously labeled with a fluorescent moiety. In some embodiments of the cell composition, the autophagy-related polypeptide is labeled with a fluorescent protein.

In some embodiments of the cell composition, the autophagy-related polypeptide is a fusion protein comprising a fluorescent protein. In some embodiments of the cell composition, the autophagy-related polypeptide is a LC3 polypeptide. In some embodiments of the cell composition, the LC3 polypeptide is labeled with a fluorescent moiety. In some embodiments of the cell composition, the LC3 polypeptide is labeled with a fluorescent protein.

In some embodiments of the cell composition, the GFP-LC3 fusion polypeptide comprises a LC3-II polypeptide. In some embodiments of the cell composition, the GFP-LC3 fusion polypeptide comprises about 80%, 85%, 90%, 95%, 96%, 97%, 98%, or 99% sequence identity to a LC3 sequence as set forth in NCBI Accession number: NP 115903.1. In some embodiments of the cell composition, the GFP-LC3 fusion polypeptide comprises about 95%, 96%, 97%, 98%, or 99% sequence identity to a LC3 sequence as set forth in NCBI Accession number: NP 115903.1.

In some embodiments of the cell composition, the UPS nanoparticle solution comprises a first population of micelles, wherein the first population of micelles comprises a first block copolymer, wherein the first block copolymer is a block copolymer of Formula I or a block copolymer of Formula II. In some embodiments of the cell composition, the UPS nanoparticle solution comprises a first population of micelles, wherein the first population of micelles comprises a first block copolymer, wherein the first block copolymer is a block copolymer of Formula I. In some embodiments of the cell composition, the UPS nanoparticle solution comprises a first population of micelles, wherein the first population of micelles comprises a first block copolymer, wherein the first block copolymer is a block copolymer of Formula II.

In some embodiments of the cell composition, the first block copolymer is a block copolymer of Formula (I):

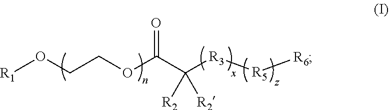

### Wherein:

R₁ is hydrogen, alkyl_{(c≤12)}, cyloalkyl_{(c≤12)}, substituted alkyl_{(c≤12)}, substituted cyloalkyl_{(c≤12)}, or

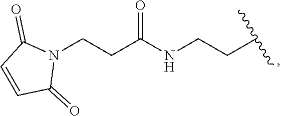 [SMILES: *CCNC(=O)CCN1C(=O)C=CC1=O]

or a metal chelating group;

R₂ and R₂′ are each independently selected from hydrogen, alkyl_{(c≤12)}, cyloalkyl_{(c≤12)}, substituted alkyl_{(c≤12)}, or substituted cyloalkyl_{(c≤12)};

n is an integer from 1 to 500;

R₃ is a group of the formula (Ia):

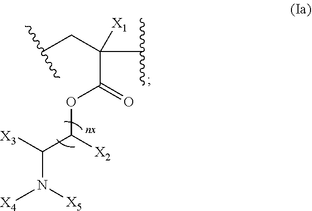

wherein:

nₓ is 1-10;

X₁, X₂, and X₃ are each independently selected from hydrogen, alkyl_{(c≤12)}, cycloalkyl_{(c≤12)}, substituted alkyl_{(c≤12)}, or substituted cycloalkyl_{(c≤12)}; and

X₄ and X₅ are each independently selected from alkyl_{(c≤12)}, cycloalkyl_{(c≤12)}, aryl_{(c≤12)}, heteroaryl(C₁₂, substituted alkyl_{(c≤12)}, substituted cycloalkyl_{(c≤12)}, substituted aryl_{(c≤12)}, or substituted heteroaryl_{(c≤12)}; or X₄ and X₅ are taken together and are alkanediyl_{(c≤12)}, alkoxydiyl_{(c≤12)}, alkylaminodiyl_{(c≤12)}, substituted alkanediyl_{(c≤12)}, substituted alkoxydiyl_{(c≤12)}, or substituted alkylaminodiyl_{(c≤12)};

x is an integer from 1 to 150;

R₅ is a group of the Formula (Ib):

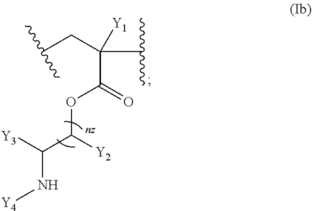

wherein:

n_{z} is 1-10;

Y₁, Y₂, and Y₃ are each independently selected from hydrogen, alkyl_{(c≤12)}, cycloalkyl_{(c≤12)}, substituted alkyl_{(c≤12)}, or substituted cycloalkyl_{(c≤12)}; and Y₄ is hydrogen, alkyl_{(c≤12)}, acyl_{(c≤12)}, substituted alkyl_{(c≤12)}, substituted acyl_{(c≤12)}, a dye, or a fluorescence quencher;

z is an integer from 0-6; and

R₆ is hydrogen, halo, hydroxy, alkyl_{(c≤12)}, or substituted alkyl_{(c≤12)},

wherein R₃ and R₅ can occur in any order within the polymer.

In some embodiments of the cell composition, the first block copolymer is a block copolymer of Formula II:

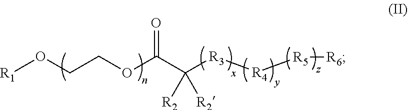

wherein:

R₁ is hydrogen, alkyl_{(c≤12)}, cycloalkyl_{(c≤12)}, substituted alkyl_{(c≤12)}, substituted cycloalkyl_{(c≤12)}, or

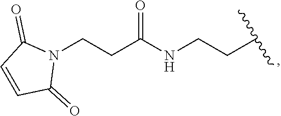 [SMILES: *CCNC(=O)CCN1C(=O)C=CC1=O]

or a metal chelating group;

n is an integer from 1 to 500;

R₂ and R₂′ are each independently selected from hydrogen, alkyl_{(c≤12)}, cycloalkyl_{(c≤12)}, substituted alkyl_{(c≤12)}, or substituted cycloalkyl_{(c≤12)};

R₃ is a group of the Formula (IIa):

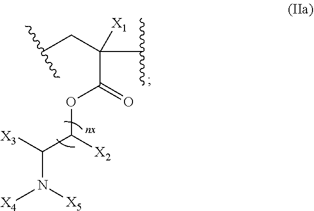

wherein:

nₓ is 1-10;

X₁, X₂, and X₃ are each independently selected from hydrogen, alkyl_{(c≤12)}, cycloalkyl_{(c≤12)}, substituted alkyl_{(c≤12)}, or substituted cycloalkyl_{(c≤12)}; and X₄ and X₅ are each independently selected from alkyl_{(c≤12)}, cycloalkyl_{(c≤12)}, aryl_{(c≤12)}, heteroaryl_{(c≤12)}, substituted alkyl_{(c≤12)}, substituted cycloalkyl_{(c≤12)}, substituted aryl_{(c≤12)}, or substituted heteroaryl_{(c≤12)}; or X₄ and X₅ are taken together and are alkanediyl_{(c≤12)}, alkoxydiyl_{(c≤12)}, alkylaminodiyl_{(c≤12)}, substituted alkanediyl_{(c≤12)}, substituted alkoxydiyl_{(c≤12)}, or substituted alkylaminodiyl_{(c≤12)};

x is an integer from 1 to 150;

R₄ is a group of the formula (IIb):

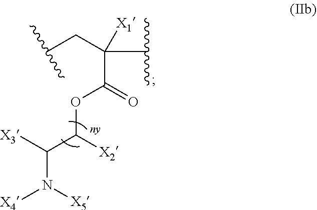

wherein:

n_{y} is 1-10;

X₁′, X₂′, and X₃′ are each independently selected from hydrogen, alkyl_{(c≤12)}, cycloalkyl_{(c≤12)}, substituted alkyl_{(c≤12)}, or substituted cycloalkyl_{(c≤12)}; and X₄′ and X₅′ are each independently selected from alkyles_{(c≤12)}, cycloalkyl_{(c≤12)}, aryl_{(c≤12)}, heteroaryl(C₁₂, substituted alkyl_{(c≤12)}, substituted cycloalkyl_{(c≤12)}, substituted aryl_{(c≤12)}, or substituted heteroaryl_{(c≤12)}; or X₄′ and X₅′ are taken together and are alkanediyl_{(c≤12)}, alkoxydiyl_{(c≤12)}, alkylaminodiyl_{(c≤12)}, substituted alkanediyl_{(c≤12)}, substituted alkoxydiyl_{(c≤12)}, or substituted alkylaminodiyl_{(c≤12)};

y is an integer from 1 to 150;

R₅ is a group of the Formula (IIc):

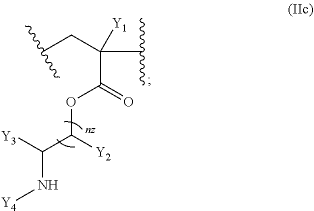

wherein:

n_{z} is 1-10;

Y₁, Y₂, and Y₃ are each independently selected from hydrogen, alkyl_{(c≤12)}, cycloalkyl_{(c≤12)}, substituted alkyl_{(c≤12)}, or substituted cycloalkyl_{(c≤12)}; and Y₄ is hydrogen, alkyl_{(c≤12)}, acyl_{(c≤12)}, substituted alkyl_{(c≤12)}, substituted acyl(2), a dye, or a fluorescence quencher;

z is an integer from 0-6; and

R₆ is hydrogen, halo, hydroxy, alkyl_{(c≤12)}, or substituted alkyl_{(c≤12)},

wherein R₃, R₄, and R₅ can occur in any order within the polymer, provided that R₃ and R₄ are not the same group.

In some embodiments of the cell composition, R₁ is alkyl_{(c≤6)}. In some embodiments of the cell composition, R₁ is methyl.

In some embodiments of the cell composition, R₂ is alkyl_{(c≤6)}. In some embodiments of the cell composition, R₂ is methyl.

In some embodiments of the composition, R₂′ is alkyl_{(c≤6)}. In some embodiments of the composition, R₂′ is methyl.

In some embodiments of the composition, R₃ is

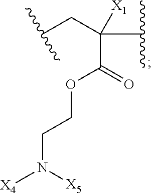

wherein X₁ is selected from hydrogen, alkyl_{(c≤8)}, or substituted alkyl_{(c≤8)}; and X₄ and X₅ are each independently selected from alkyl_{(c≤12)}, aryl_{(c≤12)}, heteroaryl_{(c≤12)}, substituted alkyl_{(c≤12)}, substituted aryl_{(c≤12)}, or substituted heteroaryl_{(c≤12)}; or X₄ and X₅ are taken together and are alkanediyl_{(c≤8)} or substituted alkanediyl_{(c≤8)}. In some embodiments of the composition, X₁ is alkyl_{(c≤6)}. In some embodiments of the composition, X₁ is methyl. In some embodiments of the method of screening, X₄ is alkyl_{(c≤8)}. In some embodiments of the composition, X₄ is methyl, ethyl, propyl, butyl, or pentyl. In some embodiments of the composition, X₅ is alkyl_{(c≤8)}. In some embodiments of the composition, X₅ is methyl, ethyl, propyl, butyl, or pentyl. In some embodiments of the composition, X₄ and X₅ are taken together and are alkanediyl(s) or substituted alkanediyl_{(c≤8)}.

In some embodiments of the composition, R₄ is

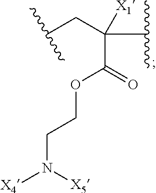

wherein X₁′ is selected from hydrogen, alkyl_{(c≤8)}, or substituted alkyl_{(c≤8)}; and X₄′ and X₅′ are each independently selected from alkyl_{(c≤12)}, aryl_{(c≤12)}, heteroaryl_{(c≤12)}, substituted alkyl_{(c≤12)}, substituted aryl_{(c≤12)}, or substituted heteroaryl_{(c≤12)}; or X₄′ and X₅′ are taken together and are alkanediyl_{(c≤8)} or substituted alkanediyl_{(c≤8)}. In some embodiments of the composition, X₁′ is alkyl_{(c≤6)}. In some embodiments of the composition, X₁ is methyl. In some embodiments of the composition, X₄′ is alkyl_{(c≤8)}. In some embodiments of the composition, X₄′ is methyl, ethyl, propyl, butyl, or pentyl. In some embodiments of the composition, X₅′ is alkyl_{(c≤8)}. In some embodiments of the composition, X₅′ is methyl, ethyl, propyl, butyl, or pentyl. In some embodiments of the method of composition, X₄′ and X₅′ are taken together and are alkanediyl_{(c≤8)} or substituted alkanediyl_{(c≤8)}.

In some embodiments of the composition, each R₃ is incorporated consecutively to form a block. In some embodiments of the composition, each R₄ is incorporated consecutively to form a block. In some embodiments of the composition, R₃ is present as a block and R₄ is present as a block. In some embodiments of the composition, R₃ and R₄ are randomly incorporated within the polymer.

In some embodiments of the composition, R₅ is

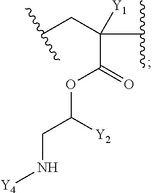

wherein Y₁ is selected from hydrogen, alkyl_{(c≤8)}, substituted alkyl_{(c≤8)}; and Y₄ is hydrogen, a dye, or a fluorescence quencher. In some embodiments of the method of screening, Y₁ is alkyl_{(c≤8)}, alkyl_{(c≤7)}, alkyl_{(c≤6)}, alkyl_{(c≤5)}, alkyl_{(c≤4)}, alkyl_{(c≤3)}, or alkyl_{(c≤2)}. In some embodiments of the composition, Y₁ is methyl.

In some embodiments of the composition, Y₄ is hydrogen or a dye. In some embodiments of the composition, Y₄ is hydrogen. In some embodiments of the composition, Y₄ is a dye. In some embodiments of the methods of screening, Y₄ is a fluorescent dye. In some embodiments of the composition, the fluorescent dye is a fluorescein, rhodamine, xanthene, BODIPY®, Alexa Fluor®, or a cyanine dye. In some embodiments of the composition, the fluorescent dye of Y₄ is indocyanine green, AMCA-x, Marina Blue, PyMPO, Rhodamine Green™, Tetramethylrhodamine, 5-carboxy-X-rhodamine, Bodipy493, Bodipy TMR-x, Bodipy630, Cyanine3.5, Cyanine5, Cyanine5.5, or Cyanine7.5. In some embodiments of the composition, the fluorescent dye is indocyanine green.

In some embodiments of the composition, Y₄ is a fluorescence quencher. In some embodiments of the composition, the fluorescence quencher is QSY7, QSY21, QSY35, BHQ1, BHQ2, BHQ3, TQ1, TQ2, TQ3, TQ4, TQ5, TQ6, or TQ7. In some embodiments of the composition, n is an integer from 75 to 150. In some embodiments of the methods of screening, n is an integer from 100 to 125.

In some embodiments of the composition, x is 1-120. In some embodiments of the composition, x is from 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, or any range derivable therein.

In some embodiments of the composition, y is 1-120. In some embodiments of the composition, y is from 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, or any range derivable therein.

In some embodiments of the composition, z is 0-6. In some embodiments of the methods of screening, z is 1-6. In some embodiments of the composition, z is from 0-2, 2-4, 4-6, or any range derivable therein.

In some embodiments of the composition; R₃, R₄, and R₅ occur in any order within the polymer. In some embodiments of the composition; R₃, R₄, and R₅ occur in any order described in Formula (II).

In some embodiments of the composition, R₃ is

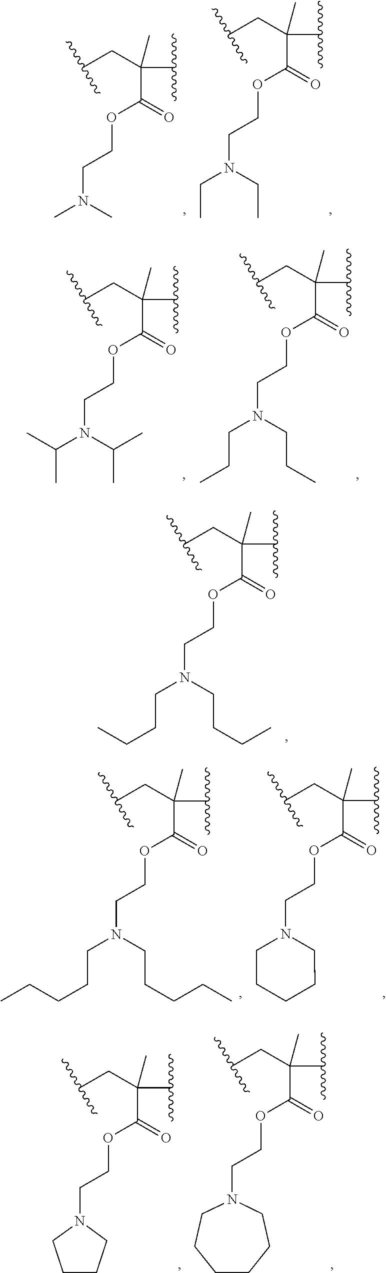

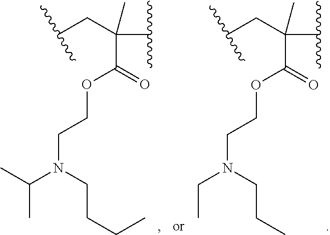 [SMILES: *CC(*)(C)C(=O)OCCN(CCCC)C(C)C ; *CC(*)(C)C(=O)OCCN(CC)CCC]

In some embodiments of the composition, R₄ is

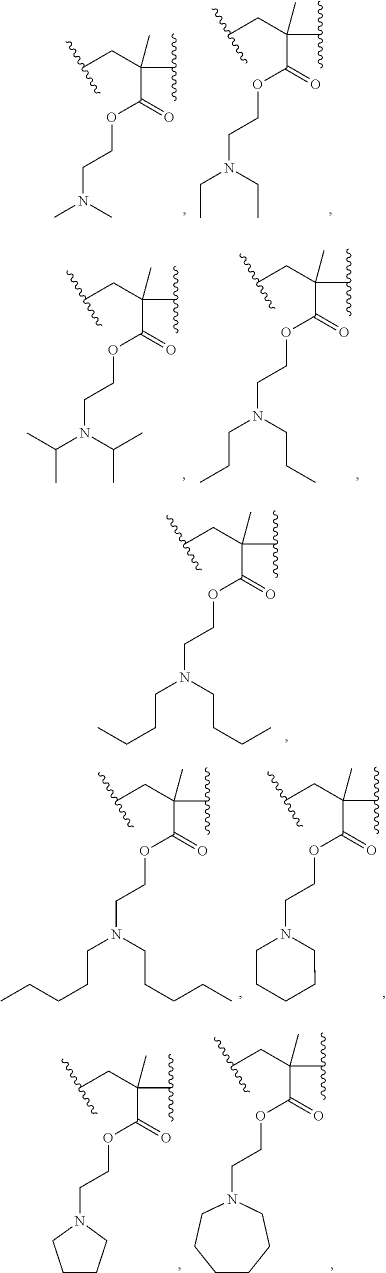

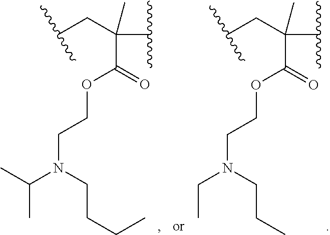 [SMILES: *CC(*)(C)C(=O)OCCN(CCCC)C(C)C ; *CC(*)(C)C(=O)OCCN(CC)CCC]

In some embodiments of the composition, R₃ is presented as a block and R₅ is presented as a block. In some embodiments of the composition, R₃ and R₅ are randomly incorporated into the polymer. In some embodiments of the composition, R₃ and R₅ occur in the order as described in Formula (I).

In some embodiments of the cell composition, the first population of micelles further comprises a second block copolymer, wherein the second block copolymer is a block copolymer of Formula I or a block copolymer of Formula II. In some embodiments of the cell composition, the first population of micelles further comprises a second block copolymer, wherein the second block copolymer is a block copolymer of Formula I. In some embodiments of the cell composition, the first population of micelles further comprises a second block copolymer, wherein the second block copolymer is a block copolymer of Formula II.

In some embodiments of the cell composition, the UPS nanoparticle solution further comprises a second population of micelles, wherein the second population of micelles comprises a block copolymer of Formula I or a block copolymer of Formula II. In some embodiments of the cell composition, the UPS nanoparticle solution further comprises a second population of micelles, wherein the second population of micelles comprises a block copolymer of Formula I. In some embodiments of the cell composition, the UPS nanoparticle solution further comprises a second population of micelles, wherein the second population of micelles comprises a block copolymer of Formula II.

In some embodiments of the composition, the molecule is covalently attached to the block copolymer. In some embodiments of the composition, the molecule is non-covalently attached to the block copolymer.

In some embodiments of the composition, the block copolymer comprises poly(acrylic acid) (PAA); poly(methyl acrylate) (PMA); polystyrene (PS); poly(ethylene oxide) (PEO) or poly(ethylene glycol); poly(butadiene) (PBD); poly(buylene oxide) (PBO); poly(2-methyloxazoline) (PMOXA); poly(dimethyl siloxane) (PDMS); poly(e-caprolactone) (PCL); poly(propylene sulpide) (PPS); poly(N-isopropylacrylamide) (PNIPAM); poly(2-vinylpyridine) (P2VP); poly(2-(diethylamino)ethyl methacrylate) (PDEA); poly(2-(diisopropylamino)ethyl methacrylate) (PDPA); poly(2-(methacryloyloxy)ethyl phosphorylcholine) (PMPC); poly(lactic acid)) (PLA); a derivative thereof, or a combination thereof. In some embodiments of the composition, the block copolymer comprises poly(ethylene oxide) (PEO) or poly(ethylene glycol). In some embodiments of the composition, the block copolymer comprises poly(lactic acid)) (PLA). In some embodiments of the composition, the block copolymer is PEG-PLA.

In some embodiments of the composition, the molecule is a small molecule compound. In some embodiments of the composition, the molecule is a protein or a peptide. In some embodiments of the composition, the molecule is a peptidomimetic. In some embodiments of the composition, the molecule is a polynucleotide.

In some embodiments of the composition, the molecule is digoxin (DG), proscillaridin A, digoxigenin, alexidine dihydrochloride (AD), cycloheximide, ikarugamycin (SW201073; IKA), or a derivative thereof. In some embodiments of the composition, the molecule is digoxin (DG), alexidine dihydrochloride (AD), ikarugamycin (SW201073; IKA), or a derivative thereof. In some embodiments of the composition, the molecule is digoxin (DG) or a derivative thereof. In some embodiments of the composition, the molecule is alexidine dihydrochloride (AD) or a derivative thereof. In some embodiments of the composition, the molecule is ikarugamycin (SW201073; IKA) or a derivative thereof.

### VII. METHODS OF SCREENING

Also disclosed herein in certain embodiments are methods of screening for an agonist of a basic helix-loop-helix leucine zipper transcriptional factor of the microphthalmia-associated transcription factor (MITF)/transcriptional factor E (TFE) family (MiT), comprising:

a) incubating a cell expressing a fluorescent-labeled autophagy-related polypeptide with a UPS nanoparticle solution for a first time period sufficient for an autophagy-associated organelle within the cell to uptake the UPS nanoparticle;

b) contacting the UPS nanoparticle-treated cell with a molecule for a second time period sufficient for the cell to uptake the molecule;

c) measuring a fluorescence signal of the fluorescent-labeled autophagy-related polypeptide; and

d) comparing the fluorescence signal with a control, wherein a decrease in fluorescence signal indicates the molecule is an agonist against a basic helix-loop-helix leucine zipper transcriptional factor of the MITF/TFE family.

Disclosed herein in certain embodiments are methods of screening for an agonist of a basic helix-loop-helix leucine zipper transcriptional factor of the microphthalmia-associated transcription factor (MITF)/transcriptional factor E (TFE) family (MiT), comprising:

a) incubating a cell expressing a fluorescent-labeled LC3 polypeptide with a UPS nanoparticle solution for a first time period sufficient for an autophagosome within the cell to uptake the UPS nanoparticle;

b) contacting the UPS nanoparticle-treated cell with a molecule for a second time period sufficient for the cell to uptake the molecule;

c) measuring a fluorescence signal of the fluorescent-labeled LC3 polypeptide; and

d) comparing the fluorescence signal with a control, wherein a decrease in fluorescence signal indicates the molecule has an agonist activity against a basic helix-loop-helix leucine zipper transcriptional factor of the MITF/TFE family.

In some embodiments of the methods of screening, the basic helix-loop-helix leucine zipper transcriptional factor of the MITF/TFE family is transcription factor EB (TFEB), transcription factor E3 (TFE3), transcription factor EC (TFEC), or microphthalmia-associated transcription factor (MITF). In some embodiment of the methods of screening, the basic helix-loop-helix leucine zipper transcriptional factor of the MITF/TFE family is transcription factor EB (TFEB). In some embodiment of the method of screening, the basic helix-loop-helix leucine zipper transcriptional factor of the MITF/TFE family is transcription factor E3 (TFE3).

In some embodiments of the methods of screening, the UPS nanoparticle solution has a buffering capacity of from about pH 4.4 to about pH 4.7. In some embodiments of the methods of screening, the buffering capacity is about pH 4.4, about pH 4.5, about pH 4.6, or about pH 4.7. In some embodiment of the method of screening, the buffering capacity is about 4.4. In some embodiments of the methods of screening, the buffering capacity is about 4.7.

In some embodiments of the methods of screening, the autophagy-associated organelle comprises autophagosome, amphisome, phagophore, endosome, or lysosome. In some embodiments of the methods of screening, the autophagy-associated organelle comprises autophagosome, amphisome, endosome, or lysosome. In some embodiments of the method of screening, the autophagy-associated organelle comprises autophagosome. In some embodiments of the method of screening, the autophagy-associated organelle comprises amphisome.

In some embodiments of a methods of screening, the UPS nanoparticle solution inhibits the formation of autolysosome by the autophagosome and/or amphisome. In some embodiments of a methods of screening, the UPS nanoparticle solution inhibits the formation of autolysosome by the autophagosome. In some embodiments of a methods of screening, the UPS nanoparticle solution inhibits the formation of autolysosome by the amphisome.

In some embodiments of the methods of screening, the molecule overrides the inhibitory activity of the UPS nanoparticle by inducing activation of TFEB and/or TFE3. In some embodiments of the methods of screening, the molecule overrides the inhibitory activity of the UPS nanoparticle by inducing activation of TFEB. In some embodiments of the methods of screening, the molecule overrides the inhibitory activity of the UPS nanoparticle by inducing activation of TFE3.

In some embodiments of the methods of screening, the molecule is a small molecule compound, a protein, a peptidomimetic, or a polynucleotide. In some embodiments of the methods of screening, the molecule is a small molecule compound. In some embodiments of the methods of screening, the molecule is a protein. In some embodiments of the methods of screening, the molecule is a peptidomimetic. In some embodiments of the methods of screening, the molecule is a polynucleotide.

In some embodiments of the methods of screening, the fluorescent-labeled autophagy-related polypeptide comprises LC3, p62, NBR1, or NDP52. In some embodiments of the methods of screening, the fluorescent-labeled autophagy-related polypeptide comprises LC3.

In some embodiments of the methods of screening, the fluorescent-labeled autophagy-related polypeptide comprises a fluorescent moiety. In some embodiments of the methods of screening, the fluorescent moiety comprises a fluorescent molecule or a fluorescent protein. In some embodiments of the methods of screening, the fluorescent-labeled autophagy-related polypeptide comprises a fluorescent protein. In some embodiments of the methods of screening, the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyanl, Midori-Ishi Cyan, TagCFP, mTFP1, enhanced yellow fluorescent protein (EYFP), Topaz, MCitrine, YPet, TagYFP, PhiYFP, ZsYellow 1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRed1, mRaspberry, dKeima-Tandem, mPlum, or AQ143.

In some embodiments of the methods of screening, the autophagy-related polypeptide is exogenously labeled with a fluorescent moiety. In some embodiments of the methods of screening, the autophagy-related polypeptide is labeled with a fluorescent protein.

In some embodiments of the methods of screening, the autophagy-related polypeptide is a fusion protein comprising a fluorescent protein. In some embodiments of the methods of screening, the autophagy-related polypeptide is a LC3 polypeptide. In some embodiments of the methods of screening, the LC3 polypeptide is labeled with a fluorescent moiety. In some embodiments of the methods of screening, the LC3 polypeptide is labeled with a fluorescent protein. In some embodiments of the methods of screening, the fluorescent-labeled LC3 polypeptide is a GFP-LC3 fusion polypeptide. In some embodiments of the method of screening, the GFP-LC3 fusion polypeptide comprises a LC3-II polypeptide. In some embodiments of the methods of screening, the GFP-LC3 fusion polypeptide comprises about 80%, 85%, 90%, 95%, 96%, 97%, 98%, or 99% sequence identity to a LC3 sequence as set forth in NCBI Accession number: NP 115903.1 In some embodiments of the methods of screening, the GFP-LC3 fusion polypeptide comprises about 95%, 96%, 97%, 98%, or 99% sequence identity to a LC3 sequence as set forth in NCBI Accession number: NP 115903.1

In some embodiments of the methods of screening, the first time period is from about 1 hour to about 36 hours, from about 2 hours to about 32 hours, from about 5 hours to about 24 hours, from about 8 hours to about 18 hours, from about 10 hours to about 15 hours, from about 8 hours to about 24 hours, or from about 12 hours to about 18 hours. In some embodiments of the method of screening, the first time period is at least 1 hour, 2 hours, 3 hours, 5 hours, 6 hours, 7 hours, 8 hours, 9 hours, 10 hours, 12 hours, 18 hours, 24 hours, 36 hours, or more. In some embodiments of the method of screening, the first time period is at least 1 hour. In some embodiments of the method of screening, the first time period is at least 2 hours. In some embodiments of the method of screening, the first time period is at least 3 hours. In some embodiments of the method of screening, the first time period is at least 5 hours. In some embodiments of the method of screening, the first time period is at least 6 hours. In some embodiments of the method of screening, the first time period is at least 7 hours. In some embodiments of the method of screening, the first time period is at least 8 hours. In some embodiments of the method of screening, the first time period is at least 9 hours. In some embodiments of the method of screening, the first time period is at least 10 hours. In some embodiments of the method of screening, the first time period is at least 12 hours. In some embodiments of the method of screening, the first time period is at least 18 hours. In some embodiments of the method of screening, the first time period is at least 24 hours. In some embodiments of the method of screening, the first time period is at least 36 hours. In some embodiments of the method of screening, the first time period is about 1 hour. In some embodiments of the method of screening, the first time period is about 2 hours. In some embodiments of the method of screening, the first time period is about 3 hours. In some embodiments of the method of screening, the first time period is about 5 hours. In some embodiments of the method of screening, the first time period is about 6 hours. In some embodiments of the method of screening, the first time period is about 7 hours. In some embodiments of the method of screening, the first time period is about 8 hours. In some embodiments of the method of screening, the first time period is about 9 hours. In some embodiments of the method of screening, the first time period is about 10 hours. In some embodiments of the method of screening, the first time period is about 12 hours. In some embodiments of the method of screening, the first time period is about 18 hours. In some embodiments of the method of screening, the first time period is about 24 hours. In some embodiments of the method of screening, the first time period is about 36 hours.

In some embodiments of the methods of screening, the second time period is at least 30 minutes, at least 1 hour, at least 2 hours, at least 3 hours, at least 4 hours, at least 5 hours, at least 6 hours, or more. In some embodiments of the methods of screening for transcription factor EB (TFEB) or E3 (TFE3) agonist, the second time period is at least 30 minutes. In some embodiments of the methods of screening for transcription factor EB (TFEB) or E3 (TFE3) agonist, the second time period is at least 1 hour. In some embodiments of the methods of screening for transcription factor EB (TFEB) or E3 (TFE3) agonist, the second time period is at least 2 hours. In some embodiments of the methods of screening for transcription factor EB (TFEB) or E3 (TFE3) agonist, the second time period is at least 3 hours. In some embodiments of the methods of screening for transcription factor EB (TFEB) or E3 (TFE3) agonist, the second time period is at least 4 hours. In some embodiments of the methods of screening for transcription factor EB (TFEB) or E3 (TFE3) agonist, the second time period is at least 5 hours. In some embodiments of the methods of screening for transcription factor EB (TFEB) or E3 (TFE3) agonist, the second time period is at least 6 hours. In some embodiments of the methods of screening for transcription factor EB (TFEB) or E3 (TFE3) agonist, the second time period is about 30 minutes. In some embodiments of the methods of screening for transcription factor EB (TFEB) or E3 (TFE3) agonist, the second time period is about 1 hour. In some embodiments of the methods of screening for transcription factor EB (TFEB) or E3 (TFE3) agonist, the second time period is about 2 hours. In some embodiments of the methods of screening for transcription factor EB (TFEB) or E3 (TFE3) agonist, the second time period is about 3 hours. In some embodiments of the methods of screening for transcription factor EB (TFEB) or E3 (TFE3) agonist, the second time period is about 4 hours. In some embodiments of the methods of screening for transcription factor EB (TFEB) or E3 (TFE3) agonist, the second time period is about 5 hours. In some embodiments of the methods of screening for transcription factor EB (TFEB) or E3 (TFE3) agonist, the second time period is about 6 hours.

In some embodiments of the methods of screening, the control is an equivalent cell comprising a fluorescent-labeled autophagy-related polypeptide incubated with a UPS nanoparticle solution in the absence of the molecule.

In some embodiments of the methods of screening, the cell is from a mammal. In some embodiments of the methods of screening, the cell is from a human.

In some embodiments of the methods of screening, the molecule is identified as an agonist if the molecule promotes dephosphorylation of TFEB and/or TFE3, optionally through the calcium/calmodulin-dependent dephosphorylation by calcineurin protein phosphatase. In some embodiments of the methods of screening, the molecule is identified as an agonist if the molecule inhibits mTORC1 or the mTORC1 pathway. In some embodiments of the methods of screening, the molecule is identified as an agonist if the molecule inhibits the 5′-adenosine monophosphate-activated protein kinase (AMPK)—mammalian target of rapamycin (mTOR) pathway. In some embodiments of the methods of screening, the molecule is identified as an agonist if the molecule induces lysosomal, mitochondrial and/or endoplasmic reticuli (ER)-specific release of Ca²⁺. In some embodiments of the methods of screening, the molecule is identified as an agonist if the molecule is an agonist of calcineurin protein phosphatase. In some embodiments of the methods of screening, the molecule is identified as an agonist if the molecule directly or indirectly activates TFEB. In some embodiments of the methods of screening, the molecule is identified as an agonist if the molecule directly or indirectly activates TFE3.

In certain embodiments, there are methods of screening for a transcription factor EB (TFEB) agonist, comprising:

a) incubating a cell expressing a fluorescent-labeled autophagy-related polypeptide with a UPS nanoparticle solution for a first time period sufficient for an autophagy-associated organelle within the cell to uptake the UPS nanoparticle;

b) contacting the UPS nanoparticle-treated cell with a molecule for a second time period sufficient for the cell to uptake the molecule;

c) measuring a fluorescence signal of the fluorescent-labeled autophagy-related polypeptide; and

d) comparing the fluorescence signal with a control, wherein a decrease in fluorescence signal indicates the molecule is a transcription factor EB (TFEB) agonist.

In certain embodiments there are methods of screening for a transcription factor E3 (TFE3) agonist, comprising:

a) incubating a cell expressing a fluorescent-labeled autophagy-related polypeptide with a UPS nanoparticle solution for a first time period sufficient for an autophagy-associated organelle within the cell to uptake the UPS nanoparticle;

b) contacting the UPS nanoparticle-treated cell with a molecule for a second time period sufficient for the cell to uptake the molecule;

c) measuring a fluorescence signal of the fluorescent-labeled autophagy-related polypeptide; and

d) comparing the fluorescence signal with a control, wherein a decrease in fluorescence signal indicates the molecule is a transcription factor E3 (TFE3) agonist.

In some embodiments of the methods of screening for transcription factor EB (TFEB) or E3 (TFE3) agonist, the UPS nanoparticle has a buffering capacity of from about pH 4.4 to about pH 4.7. In some embodiments of the methods of screening for transcription factor EB (TFEB) or E3 (TFE3) agonist, the UPS nanoparticle solution has a buffering capacity of about pH 4.4., about pH 4.5, about pH 4.6, or about pH 4.7. In some embodiments of the methods of screening for transcription factor EB (TFEB) or E3 (TFE3) agonist, the UPS nanoparticle solution has a buffering capacity of about pH 4.4. In some embodiments of the methods of screening for transcription factor EB (TFEB) or E3 (TFE3) agonist, the UPS nanoparticle solution has a buffering capacity of about pH 4.7.

In some embodiments of the methods of screening for transcription factor EB (TFEB) or E3 (TFE3) agonist, the autophagy-associated organelle comprises autophagosome, amphisome, phagophore, endosome, or lysosome. In some embodiments of the method of screening for transcription factor EB (TFEB) or E3 (TFE3) agonist, the autophagy-associated organelle comprises autophagosome. In some embodiments of the methods of screening for transcription factor EB (TFEB) or E3 (TFE3) agonist, the autophagy-associated organelle comprises amphisome.

In some embodiments of the methods of screening for transcription factor EB (TFEB) or E3 (TFE3) agonist, the UPS nanoparticle solution inhibits the formation of autolysosome by the autophagosome and/or the amphisome. In some embodiments of the methods of screen for transcription factor EB (TFEB) or E3 (TFE3) agonist, the molecule overrides the inhibitory activity of the UPS nanoparticle by inducing activation of TFEB. In some embodiments of the methods of screen for transcription factor EB (TFEB) or E3 (TFE3) agonist, the molecule overrides the inhibitory activity of the UPS nanoparticle by inducing activation of TFE3.

In some embodiments of the methods of screening for transcription factor EB (TFEB) or E3 (TFE3) agonist, the molecule is a small molecule compound, a protein, a peptide, a peptidomimetic, or a polynucleotide. In some embodiments of the methods of screen for transcription factor EB (TFEB) or E3 (TFE3) agonist, the molecule is a small molecule compound. In some embodiments of the methods of screen for transcription factor EB (TFEB) or E3 (TFE3) agonist, the molecule is a protein or a peptide. In some embodiments of the method of screen for transcription factor EB (TFEB) or E3 (TFE3) agonist, the molecule is a peptidomimetic. In some embodiments of the method of screen for transcription factor EB (TFEB) or E3 (TFE3) agonist, the molecule is a polynucleotide.

In some embodiments of the methods of screening for transcription factor EB (TFEB) or E3 (TFE3) agonist, the fluorescent-labeled autophagy-related polypeptide comprises LC3, p62, NBR1, or NDP52. In some embodiments of the method of screen for transcription factor EB (TFEB) or E3 (TFE3) agonist, the fluorescent-labeled autophagy-related polypeptide comprises a fluorescent moiety. In some embodiments of the method of screening for transcription factor EB (TFEB) or E3 (TFE3) agonist, the fluorescent moiety comprises a fluorescent molecule or a fluorescent protein.

In some embodiments of the method of screening for transcription factor EB (TFEB) or E3 (TFE3) agonist, the fluorescent-labeled autophagy-related polypeptide comprises a fluorescent protein.

In some embodiments of the methods of screening for transcription factor EB (TFEB) or E3 (TFE3) agonist, the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyanl, Midori-Ishi Cyan, TagCFP, mTFP1, enhanced yellow fluorescent protein (EYFP), Topaz, MCitrine, YPet, TagYFP, PhiYFP, ZsYellow 1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRed1, mRaspberry, dKeima-Tandem, mPlum, or AQ143.

In some embodiments of the methods of screening for transcription factor EB (TFEB) or E3 (TFE3) agonist, the autophagy-related polypeptide is exogenously labeled with a fluorescent moiety. In some embodiments of the method of screening for transcription factor EB (TFEB) or E3 (TFE3) agonist, the autophagy-related polypeptide is labeled with a fluorescent protein. In some embodiments of the method of screening for transcription factor EB (TFEB) or E3 (TFE3) agonist, the autophagy-related polypeptide is a fusion protein comprising a fluorescent protein.

In some embodiments of the methods of screening for transcription factor EB (TFEB) or E3 (TFE3) agonist, the autophagy-related polypeptide is a LC3 polypeptide. In some embodiments of the methods of screening for transcription factor EB (TFEB) or E3 (TFE3) agonist, the LC3 polypeptide is labeled with a fluorescent moiety. In some embodiments of the method of screening for transcription factor EB (TFEB) or E3 (TFE3) agonist, the LC3 polypeptide is labeled with a fluorescent protein.

In some embodiments of the methods of screening for transcription factor EB (TFEB) or E3 (TFE3) agonist, the fluorescent-labeled LC3 polypeptide is a GFP-LC3 fusion polypeptide. In some embodiments of the methods of screening for transcription factor EB (TFEB) or E3 (TFE3) agonist, the GFP-LC3 fusion polypeptide comprises a LC3-II polypeptide. In some embodiments of the methods of screening for transcription factor EB (TFEB) or E3 (TFE3) agonist, the GFP-LC3 fusion polypeptide comprises about 80%, 85%, 90%, 95%, 96%, 97%, 98%, or 99% sequence identity to a LC3 sequence as set forth in NCBI Accession number: NP 115903.1. In some embodiments of the methods of screening for transcription factor EB (TFEB) or E3 (TFE3) agonist, the GFP-LC3 fusion polypeptide comprises about 95%, 96%, 97%, 98%, or 99% sequence identity to a LC3 sequence as set forth in NCBI Accession number: NP 115903.1

In some embodiments of the methods of screening for transcription factor EB (TFEB) or E3 (TFE3) agonist, the first time period is from about 1 hour to about 36 hours, from about 2 hours to about 32 hours, from about 5 hours to about 24 hours, from about 8 hours to about 18 hours, from about 10 hours to about 15 hours, from about 8 hours to about 24 hours, or from about 12 hours to about 18 hours. In some embodiments of the methods of screen for transcription factor EB (TFEB) or E3 (TFE3) agonist, first time period is at least 1 hour 2 hours, 3 hours, 5 hours, 6 hours, 7 hours, 8 hours, 9 hours, 10 hours, 12 hours, 18 hours, 24 hours, 36 hours, or more. In some embodiments of the method of screening, the first time period is at least 1 hour. In some embodiments of the method of screening, the first time period is at least 2 hours. In some embodiments of the method of screening, the first time period is at least 3 hours. In some embodiments of the method of screening, the first time period is at least 5 hours. In some embodiments of the method of screening, the first time period is at least 6 hours. In some embodiments of the method of screening, the first time period is at least 7 hours. In some embodiments of the method of screening, the first time period is at least 8 hours. In some embodiments of the method of screening, the first time period is at least 9 hours. In some embodiments of the method of screening, the first time period is at least 10 hours. In some embodiments of the method of screening, the first time period is at least 12 hours. In some embodiments of the method of screening, the first time period is at least 18 hours. In some embodiments of the method of screening, the first time period is at least 24 hours. In some embodiments of the method of screening, the first time period is at least 36 hours. In some embodiments of the method of screening, the first time period is about 1 hour. In some embodiments of the method of screening, the first time period is about 2 hours. In some embodiments of the method of screening, the first time period is about 3 hours. In some embodiments of the method of screening, the first time period is about hours. In some embodiments of the method of screening, the first time period is about 6 hours. In some embodiments of the method of screening, the first time period is about 7 hours. In some embodiments of the method of screening, the first time period is about 8 hours. In some embodiments of the method of screening, the first time period is about 9 hours. In some embodiments of the method of screening, the first time period is about 10 hours. In some embodiments of the method of screening, the first time period is about 12 hours. In some embodiments of the method of screening, the first time period is about 18 hours. In some embodiments of the method of screening, the first time period is about 24 hours. In some embodiments of the method of screening, the first time period is about 36 hours.

In some embodiments of the methods of screening for transcription factor EB (TFEB) or E3 (TFE3) agonist, the second time period is at least 30 minutes, 1 hour, 2 hours, 3 hours, 4 hours, 5 hours, 6 hours, or more. In some embodiments of the methods of screening for transcription factor EB (TFEB) or E3 (TFE3) agonist, the second time period is at least 30 minutes. In some embodiments of the methods of screening for transcription factor EB (TFEB) or E3 (TFE3) agonist, the second time period is at least 1 hour. In some embodiments of the methods of screening for transcription factor EB (TFEB) or E3 (TFE3) agonist, the second time period is at least 2 hours. In some embodiments of the methods of screening for transcription factor EB (TFEB) or E3 (TFE3) agonist, the second time period is at least 3 hours. In some embodiments of the methods of screening for transcription factor EB (TFEB) or E3 (TFE3) agonist, the second time period is at least 4 hours. In some embodiments of the methods of screening for transcription factor EB (TFEB) or E3 (TFE3) agonist, the second time period is at least 5 hours. In some embodiments of the methods of screening for transcription factor EB (TFEB) or E3 (TFE3) agonist, the second time period is at least 6 hours. In some embodiments of the methods of screening for transcription factor EB (TFEB) or E3 (TFE3) agonist, the second time period is about 30 minutes. In some embodiments of the methods of screening for transcription factor EB (TFEB) or E3 (TFE3) agonist, the second time period is about 1 hour. In some embodiments of the methods of screening for transcription factor EB (TFEB) or E3 (TFE3) agonist, the second time period is about 2 hours. In some embodiments of the methods of screening for transcription factor EB (TFEB) or E3 (TFE3) agonist, the second time period is about 3 hours. In some embodiments of the methods of screening for transcription factor EB (TFEB) or E3 (TFE3) agonist, the second time period is about 4 hours. In some embodiments of the methods of screening for transcription factor EB (TFEB) or E3 (TFE3) agonist, the second time period is about 5 hours. In some embodiments of the methods of screening for transcription factor EB (TFEB) or E3 (TFE3) agonist, the second time period is about 6 hours.

In some embodiments of the methods of screening for transcription factor EB (TFEB) or E3 (TFE3) agonist, the control is an equivalent cell comprising a fluorescent-labeled autophagy-related polypeptide incubated with a UPS nanoparticle solution in the absence of the molecule.

In some embodiments of the methods of screening for transcription factor EB (TFEB) or E3 (TFE3) agonist, the cell is from a mammal. In some embodiments of the methods of screening for transcription factor EB (TFEB) or E3 (TFE3) agonist, the cell is from a human.

In some embodiments of the methods of screening for transcription factor EB (TFEB) or E3 (TFE3) agonist, the molecule is identified as an agonist if the molecule promotes nuclear localization of TFEB and/or TFE3. In some embodiments of the method of screening for transcription factor EB (TFEB) or E3 (TFE3) agonist, the molecule is identified as an agonist if the molecule promotes nuclear localization of TFEB. In some embodiments of the methods of screening for transcription factor EB (TFEB) or E3 (TFE3) agonist, the molecule is identified as an agonist if the molecule promotes nuclear localization of TFE3.

In some embodiments of the methods of screening for transcription factor EB (TFEB) or E3 (TFE3) agonist, the molecule is identified as an agonist if the molecule promotes dephosphorylation of TFEB and/or TFE3, optionally through the calcium/calmodulin-dependent dephosphorylation by calcineurin protein phosphatase. In some embodiments of the methods of screening for transcription factor EB (TFEB) or E3 (TFE3) agonist, the molecule is identified as an agonist if the molecule promotes dephosphorylation of TFEB, optionally through the calcium/calmodulin-dependent dephosphorylation by calcineurin protein phosphatase. In some embodiments of the methods of screening for transcription factor EB (TFEB) or E3 (TFE3) agonist, the molecule is identified as an agonist if the molecule promotes dephosphorylation of TFE3, optionally through the calcium/calmodulin-dependent dephosphorylation by calcineurin protein phosphatase.

In some embodiments of the methods of screening for transcription factor EB (TFEB) or E3 (TFE3) agonist, the molecule is identified as an agonist if the molecule inhibits mTORC1 or the mTORC1 pathway. In some embodiments of the methods of screening for transcription factor EB (TFEB) or E3 (TFE3) agonist, the molecule is identified as an agonist if the molecule inhibits mTORC1 pathway. In some embodiments of the methods of screening for transcription factor EB (TFEB) or E3 (TFE3) agonist, the molecule is identified as an agonist if the molecule inhibits mTORC1.

In some embodiments of the methods of screen for transcription factor EB (TFEB) or E3 (TFE3) agonist, the molecule is identified as an agonist if the molecule inhibits the 5′-adenosine monophosphate-activated protein kinase (AMPK)-mammalian target of rapamycin (mTOR) pathway.

In some embodiments of the methods of screening for transcription factor EB (TFEB) or E3 (TFE3) agonist, the molecule is identified as an agonist if the molecule induces lysosomal, mitochondrial and/or endoplasmic reticuli (ER)-specific release of Ca²⁺. In some embodiments of the methods of screening for transcription factor EB (TFEB) or E3 (TFE3) agonist, the molecule is identified as an agonist if the molecule induces lysosomal-specific release of Ca²⁺. In some embodiments of the methods of screening for transcription factor EB (TFEB) or E3 (TFE3) agonist, the molecule is identified as an agonist if the molecule induces mitochondrial-specific release of Ca²⁺. In some embodiments of the methods of screening for transcription factor EB (TFEB) or E3 (TFE3) agonist, the molecule is identified as an agonist if the molecule induces endoplasmic reticuli (ER)-specific release of Ca²⁺.

In some embodiments of the methods of screening for transcription factor EB (TFEB) or E3 (TFE3) agonist, the molecule is identified as an agonist if the molecule is an agonist of calcineurin protein phosphatase.

In some embodiments of the methods of screening for transcription factor EB (TFEB) or E3 (TFE3) agonist, the molecule is identified as an agonist if the molecule directly or indirectly activates TFEB. In some embodiments of the methods of screening for transcription factor EB (TFEB) or E3 (TFE3) agonist, the molecule is identified as an agonist if the molecule directly activates TFEB. In some embodiments of the methods of screening for transcription factor EB (TFEB) or E3 (TFE3) agonist, the molecule is identified as an agonist if the molecule indirectly activates TFEB.

In some embodiments of the methods of screening for transcription factor EB (TFEB) or E3 (TFE3) agonist, the molecule is identified as an agonist if the molecule directly or indirectly activates TFE3. In some embodiments of the methods of screening for transcription factor EB (TFEB) or E3 (TFE3) agonist, the molecule is identified as an agonist if the molecule directly activates TFE3. In some embodiments of the methods of screening for transcription factor EB (TFEB) or E3 (TFE3) agonist, the molecule is identified as an agonist if the molecule indirectly activates TFE3.

In some embodiments of the methods of screening, the UPS nanoparticle solution comprises a first and a second population of micelles. In some embodiments of the methods of screening, the first population of micelles comprises a first block copolymer.

In some embodiments of the methods of screening, the first block copolymer is a block copolymer of Formula (I):

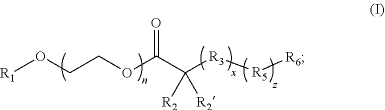

wherein:

R₁ is hydrogen, alkyl_{(c≤12)}, cyloalkyl_{(c≤12)}, substituted alkyl_{(c≤12)}, substituted cyloalkyl_{(c≤12)}, or

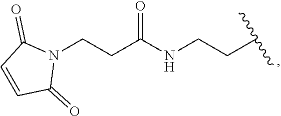 [SMILES: *CCNC(=O)CCN1C(=O)C=CC1=O]

or a metal chelating group;

R₂ and R₂′ are each independently selected from hydrogen, alkyl_{(c≤12)}, cyloalkyl_{(c≤12)}, substituted alkyl_{(c≤12)}, or substituted cyloalkyl_{(c≤12)};

n is an integer from 1 to 500;

R₃ is a group of the Formula (Ia):

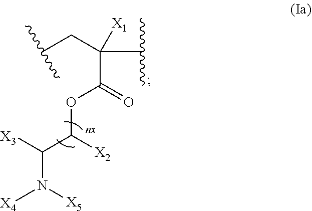

wherein:

nₓ is 1-10;

X₁, X₂, and X₃ are each independently selected from hydrogen, alkyl_{(c≤12)}, cycloalkyl_{(c≤12)}, substituted alkyl_{(c≤12)}, or substituted cycloalkyl_{(c≤12)}; and

X₄ and X₅ are each independently selected from alkyles_{(c≤12)}, cycloalkyl_{(c≤12)}, aryl_{(c≤12)}, heteroaryl_{(c≤12)}, substituted alkyl_{(c≤12)}, substituted cycloalkyl_{(c≤12)}, substituted aryl_{(c≤12)}, or substituted heteroaryle_{(c≤12)}; or X₄ and X₅ are taken together and are alkanediyl_{(c≤12)}, alkoxydiyl_{(c≤12)}, alkylaminodiyl_{(c≤12)}, substituted alkanediyl_{(c≤12)}, substituted alkoxydiyl_{(c≤12)}, or substituted alkylaminodiyl_{(c≤12)};

x is an integer from 1 to 150;

R₅ is a group of the Formula (Ib):

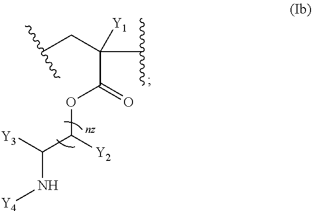

wherein:

n_{z} is 1-10;

Y₁, Y₂, and Y₃ are each independently selected from hydrogen, alkyl_{(c≤12)}, cycloalkyl_{(c≤12)}, substituted alkyl_{(c≤12)}, or substituted cycloalkyl_{(c≤12)}; and

Y₄ is hydrogen, alkyl_{(c≤12)}, acyl_{(c≤12)}, substituted alkyl_{(c≤12)}, substituted acyl_{(c≤12)}, a dye, or a fluorescence quencher;

z is an integer from 0-6; and

R₆ is hydrogen, halo, hydroxy, alkyl_{(c≤12)}, or substituted alkyl_{(c≤12)},

wherein R₃ and R₅ can occur in any order within the polymer.

In some embodiments of the methods of screening, the first block copolymer is a block copolymer of Formula II:

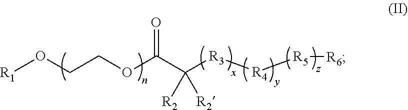

wherein:

R₁ is hydrogen, alkyl_{(c≤12)}, cycloalkyl_{(c≤12)}, substituted alkyl_{(c≤12)}, substituted cycloalkyl_{(c≤12)}, or

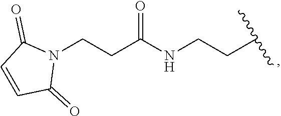 [SMILES: *CCNC(=O)CCN1C(=O)C=CC1=O]

or a metal chelating group;

n is an integer from 1 to 500;

R₂ and R₂′ are each independently selected from hydrogen, alkyl_{(c≤12)}, cycloalkyl_{(c≤12)}, substituted alkyl_{(c≤12)}, or substituted cycloalkyl_{(c≤12)};

R₃ is a group of the Formula (IIa):

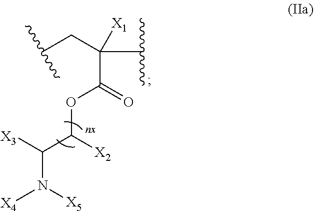

wherein:

nₓ is 1-10;

X₁, X₂, and X₃ are each independently selected from hydrogen, alkyl_{(c≤12)}, cycloalkyl_{(c≤12)}, substituted alkyl_{(c≤12)}, or substituted cycloalkyl_{(c≤12)}; and X₄ and X₅ are each independently selected from alkyl_{(c≤12)}, cycloalkyl_{(c≤12)}, aryl_{(c≤12)}, heteroaryl_{(c≤12)}, substituted alkyl_{(c≤12)}, substituted cycloalkyl_{(c≤12)}, substituted aryl_{(c≤12)}, or substituted heteroaryl_{(c≤12)}; or X₄ and X₅ are taken together and are alkanediyl_{(c≤12)}, alkoxydiyl_{(c≤12)}, alkylaminodiyl_{(c≤12)}, substituted alkanediyl_{(c≤12)}, substituted alkoxydiyl_{(c≤12)}, or substituted alkylaminodiyl_{(c≤12)};

x is an integer from 1 to 150;

R₄ is a group of the Formula (IIb):

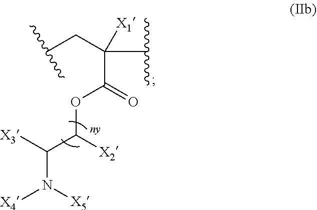

wherein:

n_{y} is 1-10;

X₁′, X₂′, and X₃′ are each independently selected from hydrogen, alkyl_{(c≤12)}, cycloalkyl_{(c≤12)}, substituted alkyl_{(c≤12)}, or substituted cycloalkyl_{(c≤12)}; and

X₄′ and X₅′ are each independently selected from alkyl_{(c≤12)}, cycloalkyl_{(c≤12)}, aryl_{(c≤12)}, heteroaryl_{(c≤12)}, substituted alkyl_{(c≤12)}, substituted cycloalkyl_{(c≤12)}, substituted aryl_{(c≤12)}, or substituted heteroaryl_{(c≤12)}; or X₄′ and X₅′ are taken together and are alkanediyl_{(c≤12)}, alkoxydiyl_{(c≤12)}, alkylaminodiyl_{(c≤12)}, substituted alkanediyl_{(c≤12)}, substituted alkoxydiyl_{(c≤12)}, or substituted alkylaminodiyl_{(c≤12)};

y is an integer from 1 to 150;

R₅ is a group of the Formula (IIc):

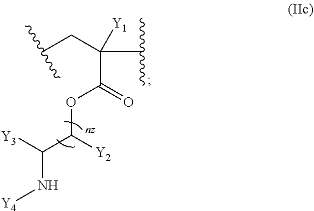

wherein:

n_{z} is 1-10;

Y₁, Y₂, and Y₃ are each independently selected from hydrogen, alkyl_{(c≤12)}, cycloalkyl_{(c≤12)}, substituted alkyl_{(c≤12)}, or substituted cycloalkyl_{(c≤12)}; and Y₄ is hydrogen, alkyl_{(c≤12)}, acyl_{(c≤12)}, substituted alkyl_{(c≤12)}, substituted acyl_{(c≤12)}, a dye, or a fluorescence quencher;

z is an integer from 0-6; and

R₆ is hydrogen, halo, hydroxy, alkyl_{(c≤12)}, or substituted alkyl_{(c≤12)},

wherein R₃, R₄, and R₅ can occur in any order within the polymer, provided that R₃ and R₄ are not the same group.

In some embodiments of the methods of screening, R₁ is alkyl_{(c≤6)}. In some embodiments of the methods of screening, R₁ is methyl, ethyl, propyl, butyl, or pentyl. In some embodiments of the methods of screening, R₁ is methyl.

In some embodiments of the methods of screening, R₂ is alkyl_{(c≤6)}. In some embodiments of the method of screening, R₂ is methyl.

In some embodiments of the methods of screening, R₂′ is alkyl_{(c≤6)}. In some embodiments of the method of screening, R₂′ is methyl.

In some embodiments of the methods of screening, R₃ is

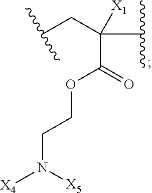

wherein X₁ is selected from hydrogen, alkyl_{(c≤8)}, or substituted alkyl_{(c≤8)}; and X₄ and X₅ are each independently selected from alkyl_{(c≤12)}, aryl_{(c≤12)}, heteroaryl_{(c≤12)}, substituted alkyl_{(c≤12)}, substituted aryl_{(c≤12)}, or substituted heteroaryl_{(c≤12)}; or X₄ and X₅ are taken together and are alkanediyl_{(c≤8)} or substituted alkanediyl_{(c≤8)}. In some embodiments of the methods of screening, X₁ is alkyl_{(c≤6)}. In some embodiments of the methods of screening, X₁ is methyl. In some embodiments of the methods of screening, X₄ is alkyl_{(c≤8)}. In some embodiments of the methods of screening, X₄ is methyl, ethyl, propyl, butyl, or pentyl. In some embodiments, X₅ is alkyl_{(c≤8)}. In some embodiments of the methods of screening, X₅ is methyl, ethyl, propyl, butyl, or pentyl. In some embodiments of the methods of screening, X₄ and X₅ are taken together and are alkanediyl_{(c≤8)} or substituted alkanediyl_{(c≤8)}.

In some embodiments of the methods of screening, R₄ is

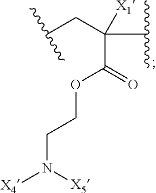

wherein X₁′ is selected from hydrogen, alkyl_{(c≤8)}, or substituted alkyl_{(c≤8)}; and X₄′ and X₅′ are each independently selected from alkyl_{(c≤12)}, aryl_{(c≤12)}, heteroaryl_{(c≤12)}, substituted alkyl_{(c≤12)}, substituted aryl_{(c≤12)}, or substituted heteroaryl_{(c≤12)}; or X₄′ and X₅′ are taken together and are alkanediyl_{(c<8)} or substituted alkanediyl_{(c<8)}. In some embodiments of the method of screening, X₁′ is alkyl_{(c≤6)}. In some embodiments of the methods of screening, X₁ is methyl. In some embodiments of the methods of screening, X₄′ is alkyl_{(C≤8)}. In some embodiments of the methods of screening, X₄′ is methyl, ethyl, propyl, butyl, or pentyl. In some embodiments of the methods of screening, X₅′ is alkyl_{(c≤8)}. In some embodiments of the methods of screening, X₅′ is methyl, ethyl, propyl, butyl, or pentyl. In some embodiments of the methods of screening, X₄′ and X₅′ are taken together and are alkanediyl_{(c≤8)} or substituted alkanediyl_{(c≤8)}.

In some embodiments of the methods of screening, each R₃ is incorporated consecutively to form a block. In some embodiments of the methods of screening, each R₄ is incorporated consecutively to form a block. In some embodiments of the methods of screening, R₃ is present as a block and R₄ is present as a block. In some embodiments of the methods of screening, R₃ and R₄ are randomly incorporated within the polymer.

In some embodiments of the methods of screening, R₅ is

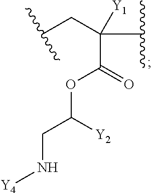

wherein Y₁ is selected from hydrogen, alkyl_{(c≤8)}, substituted alkyl_{(c≤8)}; and Y₄ is hydrogen, a dye, or a fluorescence quencher. In some embodiments of the methods of screening, Y₁ is alkyl_{(c≤8)}, alkyl_{(c≤7)}, alkyl_{(c≤6)}, alkyl_{(c≤5)}, alkyl_{(c≤4)}, alkyl_{(c≤3)}, or alkyl_{(c≤2)}. In some embodiments of the methods of screening, Y₁ is methyl.

In some embodiments of the methods of screening, Y₄ is hydrogen or a dye. In some embodiments of the method of screening, Y₄ is hydrogen. In some embodiments of the methods of screening, Y₄ is a dye. In some embodiments of the methods of screening, Y₄ is a fluorescent dye. In some embodiments of the methods of screening, the fluorescent dye is a fluorescein, rhodamine, xanthene, BODIPY®, Alexa Fluor®, or a cyanine dye. In some embodiments of the methods of screening, the fluorescent dye of Y₄ is indocyanine green, AMCA-x, Marina Blue, PyMPO, Rhodamine Green™, Tetramethylrhodamine, 5-carboxy-X-rhodamine, Bodipy493, Bodipy TMR-x, Bodipy630, Cyanine3.5, Cyanine5, Cyanine5.5, or Cyanine7.5. In some embodiments of the methods of screening, the fluorescent dye is indocyanine green.

In some embodiments of the methods of screening, Y₄ is a fluorescence quencher. In some embodiments of the methods of screening, the fluorescence quencher is QSY7, QSY21, QSY35, BHQ1, BHQ2, BHQ3, TQ1, TQ2, TQ3, TQ4, TQ5, TQ6, or TQ7.

In some embodiments of the methods of screening, n is an integer from 75 to 150. In some embodiments of the methods of screening, n is an integer from 100 to 125.

In some embodiments of the methods of screening, x is 1-120. In some embodiments of the methods of screening, x is from 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, or any range derivable therein.

In some embodiments of the methods of screening, y is 1-120. In some embodiments of the methods of screening, y is from 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, or any range derivable therein.

In some embodiments of the methods of screening, z is 0-6. In some embodiments of the methods of screening, z is 1-6. In some embodiments of the methods of screening, z is from 0-2, 2-4, 4-6, or any range derivable therein.

In some embodiments of the methods of screening; R₃, R₄, and R₅ occur in any order within the polymer. In some embodiments of the methods of screening; R₃, R₄, and R₅ occur in any order described in Formula (II).

In some embodiments of the methods of screening, the first block copolymer further comprises a targeting moiety. In some embodiments of the methods of screening, the targeting moiety is a small molecule, an antibody fragment, or a signaling peptide. In some embodiments of the methods of screening, the targeting moiety is a small molecule. In some embodiments of the methods of screening, the targeting moiety is an antibody fragment. In some embodiments of the methods of screening, the targeting moiety is a signaling peptide.

In some embodiments of the methods of screening, R₃ is

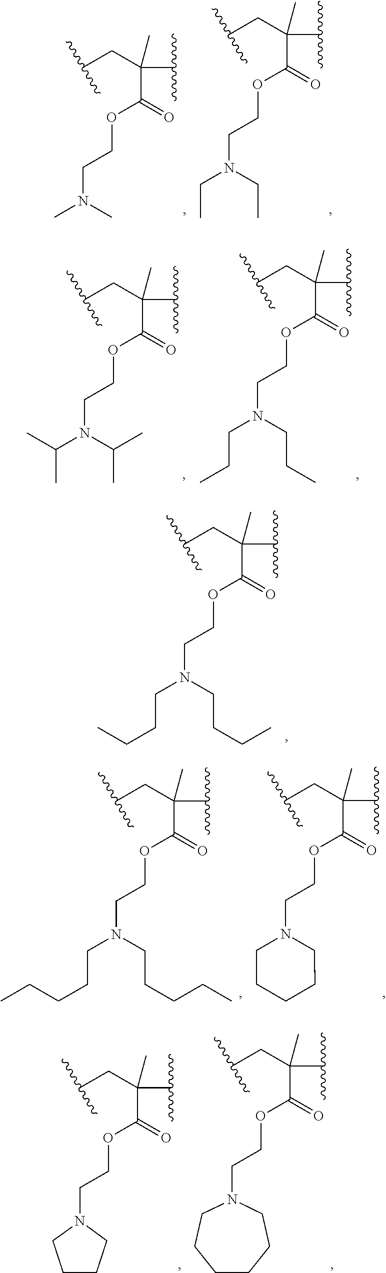

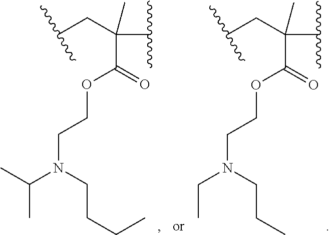 [SMILES: *CC(*)(C)C(=O)OCCN(CCCC)C(C)C ; *CC(*)(C)C(=O)OCCN(CC)CCC]

In some embodiments of the methods of screening, R₄ is

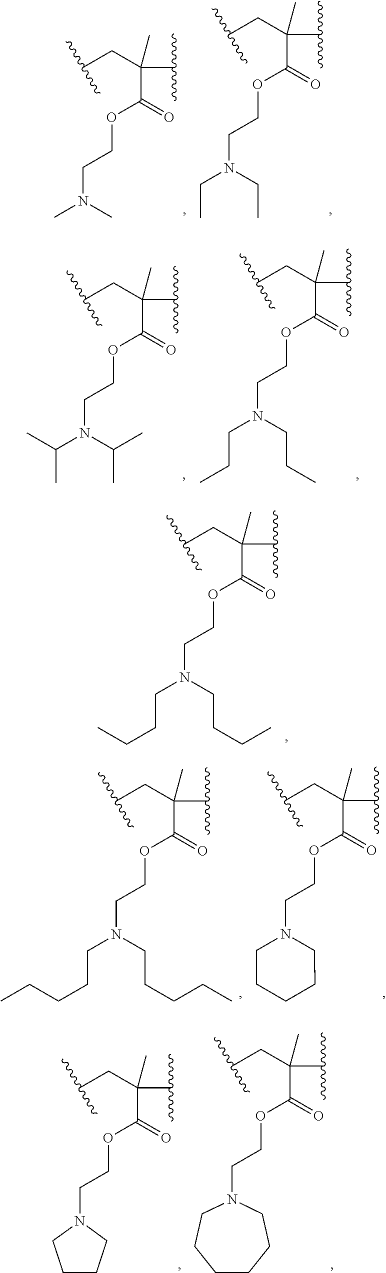

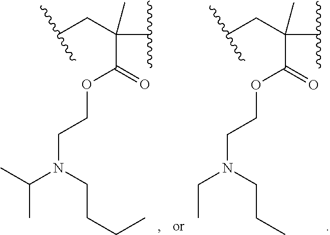 [SMILES: *CC(*)(C)C(=O)OCCN(CCCC)C(C)C ; *CC(*)(C)C(=O)OCCN(CC)CCC]

In some embodiments of the methods of screening, R₃ is presented as a block and R₅ is presented as a block. In some embodiments of the methods of screening, R₃ and R₅ are randomly incorporated into the polymer. In some embodiments of the methods of screening, R₃ and R₅ occur in the order as described in Formula (I).

In another embodiment of the methods of screening, the first population of micelles further comprises a second block copolymer. In some embodiments of the methods of screening, the second block copolymer is a copolymer of Formula (I) or a block copolymer of Formula (II). In some embodiments of the methods of screening, the second block copolymer is a copolymer of Formula (I). In some embodiments of the methods of screening, the second block copolymer is a block copolymer of Formula (II).

In some embodiments of the methods of screening, the first population of micelles has a pH response (ΔpH_{10-90%}) of less than about 1 pH unit. In some embodiments of the methods of screening, the pH response is less than about 0.30 pH units. In some embodiments of the methods of screening, the pH response is less than about 0.25 pH units. In some embodiments of the methods of screening, the pH response is less than about 0.15 pH units. In some embodiments of the methods of screening, the pH response is less than about 0.10 pH units.

In some embodiments of the methods of screening, the first population of micelles has a pH transition point of from about 3 to about 9. In some embodiments of the methods of screening, the first population of micelles has a pH transition point of from about 3 to about 8. In some embodiments of the methods of screening, the first population of micelles has a pH transition point of from about 4 to about 7. In some embodiments of the methods of screening, the first population of micelles has a pH transition point of from about 4 to about 6. In some embodiments of the methods of screening, the pH transition point is from about 4 to about 5.

In some embodiments of the methods of screening, the first population of micelles has a fluorescence signal activation ratio greater than 8. In some embodiments of the methods of screening, the first population of micelles has a fluorescence signal activation ratio greater than 9. In some embodiments of the methods of screening, the first population of micelles has a fluorescence signal activation ratio greater than 10.

In another embodiment of the methods of screening, the UPS nanoparticle solution further comprises a second population of micelles. In some embodiments of the methods of screening, the second population of micelles comprises a block copolymer of Formula (I) or a block copolymer of Formula (II). In some embodiments of the methods of screening, the second population of micelles comprises a block copolymer of Formula (I). In some embodiments of the methods of screening, the second population of micelles comprises a block copolymer of Formula (II).

### VIII. KITS

The present disclosure also provides kits. Any of the components disclosed herein may be combined in a kit. In certain embodiments the kits comprise a pH-responsive system or composition as described above.

The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the micelle populations in a series are combined in a single container. In other embodiments, some or all of the micelle population in a series are provided in separate containers.

The kits of the present disclosure also will typically include packaging for containing the various containers in close confinement for commercial sale. Such packaging may include cardboard or injection or blow molded plastic packaging into which the desired containers are retained. A kit may also include instructions for employing the kit components. Instructions may include variations that can be implemented.

### IX. METHODS OF TREATMENT

In certain embodiments, the methods, molecules, and compositions disclosed herein, or the molecules detected by the methods disclosed herein, are useful in the treatment of diseases and disorders associated with autophagosome-lysosome biogenesis.

Autophagosome-lysosome biogenesis is a catabolic process that both generates nutrients and energy during starvation and maintains homeostasis under nutrient-rich conditions. Impairment of this process is associated with metabolic disorders and ageing. In metabolic syndromes such as obesity and fatty liver disease, excess nutrients increase demand for degradative autophagy-lysosome machinery and challenge the adaptive response capacity. During ageing and within age-related disorders, a steady decline in productive autophagy impairs clearance of defective organelles leading pathological accumulation of pro-apoptotic factors and reactive oxygen species (ROS). Additionally, the autophagy-lysosome system plays an essential role in activating macrophages and other cells of the immune system in response to pathogen exposure. Macrophage activation results in drastic changes in the expression of a number of gene sets, including those responsible for inflammatory, chemoattractant, and antimicrobial effectors, among others.

As used herein, a “metabolic disease or disorder” refers to any pathological condition resulting from an alteration in a subject's metabolism. Such disorders include those resulting from an alteration in glucose homeostasis and/or insulin dysfunction. Metabolic disorders, include but are not limited to, metabolic syndrome, elevated blood glucose levels, insulin resistance, glucose intolerance, type 2 diabetes, type 1 diabetes, pre-diabetes, non-alcoholic fatty liver disease (NAFLD), nonalcoholic steatohepatitis (NASH), and obesity.

In some embodiments, the molecule that is discovered by the methods provided herein, is for the treatment of a metabolic disorder. In some embodiments, the molecule that is discovered by the methods provided herein, is for the treatment of a diabetes-related disease or disorder. In some embodiments, the molecule that is discovered by the methods provided herein, is for the treatment of type 1 diabetes, type 2 diabetes, or prediabetes.

In some embodiments, the molecule that is discovered by the methods provided herein, is for the treatment of metabolic related obesity.

In some embodiments, the molecule that is discovered by the methods provided herein, is for the treatment of nonalcoholic fatty liver disease (NAFLD). In some embodiments, the molecule that is discovered by the methods provided herein, is for the treatment of nonalcoholic steatohepatitis (NASH).

In some embodiments, there is provided a method of administering a molecule, a composition, or a method described herein to an individual with a metabolic disorder has a variety of desirable outcomes which include, but are not limited to, reducing blood glucose levels, decreasing plasma lysophosphatidic acid levels, improving insulin sensitivity, increasing insulin secretion, improving glucose tolerance, and decreasing adipose tissue expansion. Any of these outcomes can treat, delay or prevent the onset of a metabolic disorder, wherein such metabolic disorders include, but are not limited to, metabolic syndrome, elevated blood glucose levels, insulin resistance, glucose intolerance, type 2 diabetes, type 1 diabetes, pre-diabetes, non-alcoholic fatty liver disease, nonalcoholic steatohepatitis, obesity, and metabolism associated obesity.

In some embodiments, the molecule, composition, or methods disclosed herein are used to treat an underlying metabolic disorder. In some embodiments, the metabolic disorder is treated by reducing blood glucose levels, decreasing plasma lysophosphatidic acid levels, improving insulin sensitivity, increasing insulin secretion, and/or improving glucose tolerance. In some embodiments, the subject is overweight or obese. In some embodiments, the subject has type 1 diabetes, type 2 diabetes, or prediabetes. In some embodiments, the subject has non-alcoholic fatty liver disease and/or nonalcoholic steatohepatitis. In some embodiments, the subject does not have a metabolic disorder. In some embodiments, the molecule, composition, or methods disclosed herein are used to delay or prevent the onset of the metabolic disorder by reducing elevated blood glucose levels, decreasing plasma lysophosphatidic acid levels, improving insulin sensitivity, increasing insulin secretion, and/or improving glucose tolerance.

In some embodiments, the molecule, composition, or methods disclosed herein, are used for the treatment, prevention, or amelioration of an age-related disease or disorder.

During ageing and within age-related disorders, a steady decline in productive autophagy impairs clearance of defective organelles leading pathological accumulation of pro-apoptotic factors and reactive oxygen species (ROS). In some embodiments, administering a molecule, a composition, or a method described herein to an individual with an age-related disease has a variety of desirable outcomes which include, but are not limited to, reducing reactive oxygen species, enhancing lysosome function, and/or reducing pro-apoptotic factors.

In some embodiments, the molecules, composition, or methods disclosed herein, are used in used in modulating an immune response due to a pathogenic infection. In some embodiments, the pathogenic infection can result from a viral or bacterial infection.

In some embodiments, inducing secretion of key mediators of the inflammatory response, inducing macrophage differentiation, and/or inducing macrophage migration to sites of inflammation can be used to modulate an immune response.

In some embodiments, the molecules, composition, or methods disclosed herein, are used in improving the removal of defective organelles, macromolecules, and/or misfolded protein by improving lysosome function. In some embodiments, improving lysosome function will treat the metabolic disease. In some embodiments, improving lysosome function will treat the age-related disease. In dome embodiments, improving lysosome function will modulate an immune response due to a pathological infection.

In some embodiments, the molecule disclosed here is a small molecule compound, a peptide, a peptidomimetic, or a polynucleotide. In some embodiments, the molecule is a small molecule compound. In some embodiments, the molecule is a peptide or a peptidomimetic. In some embodiments, the molecule is a polynucleotide. In some embodiments, the molecule is a TFEB agonist. In some embodiments, the molecule is a TFE3 agonist.

In some embodiments, the molecules detected by the methods disclosed herein, is a small molecule compound, a peptide, a peptidomimetic, or a polynucleotide. In some embodiments, the molecules detected by the methods disclosed herein, is a small molecule compound. In some embodiments, the molecule detected by the methods disclosed herein, is a peptide or a peptidomimetic. In some embodiments, the molecules detected by the methods disclosed herein, is a polynucleotide. In some embodiments, the molecules detected by the methods disclosed herein, is a TFEB agonist.

In some embodiments, the molecule is digoxin (DG), proscillaridin A, digoxigenin, alexidine dihydrochloride (AD), cycloheximide, ikarugamycin (SW201073), or a derivative thereof. In some embodiments, the molecule is digoxin (DG), alexidine dihydrochloride (AD), ikarugamycin (SW201073; IKA), or a derivative thereof. In some embodiments, the molecule is digoxin (DG) or a derivative thereof. In some embodiments, the molecule is alexidine dihydrochloride (AD) or a derivative thereof. In some embodiments, the molecule is ikarugamycin (SW201073; IKA) or a derivative thereof.

In some embodiments, the molecule is a composition of any of the methods disclosed herein.

### X. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Chemical Reagents.

The TMR-NHS, Cy5-NHS, BODIPY-NHS and Cy3.5-NHS esters were purchased from Lumiprobe Corp. (FL, USA). 2-Aminoethyl methacrylate (AMA) was purchased from Polyscience Company. Monomers 2-(dibutylamino) ethyl methacrylate (DBA-MA) and 2-(dipentylamino) ethyl methacrylate (D5A-MA) were prepared according to the method described in the inventors' previous work, as well as the PEO macroinitiator (MeO-PEO114-Br). N,N,N′,N″,N′″-Pentamethyldiethylenetriamine (PMDETA) and poly(ethylene glycol)-b-poly(D,L-lactide) (PEG-PLA, Mn-5,000 Da for each segment) were purchased from Sigma-Aldrich. (2-Hydroxypropyl)-β-cyclodextrin (HPβCD) was purchased from Fisher Scientific Inc. Amicon ultra-15 centrifugal filter tubes (MWCO=100 K) were obtained from Millipore (MA). Other reagents and organic solvents were analytical grade from Sigma-Aldrich or Fisher Scientific Inc. Digoxin, proscillaridin A, alexdine dihydrochloride, ikarugamycin, bafilomycin A1, FK506, cyclosporine A, dorsomorphin (compound C), AICAR, metformin, STO-609, thapsigarigin, N,N,N′,N′-Tetrakis(2-pyridylmethyl)ethylenediamine (TPEN) and oleic acid were purchased from Sigma-Aldrich; Torin 1 was from Tocris Bioscience; Xestosporin C was from Cayman Chemical; BAPTA-AM, Fura-2-AM, calcium calibration buffer kit (with 0 and 10 mM EGTA), Hoechst 33342, CellROX green, tert-Butyl hydroperoxide (TBHP) and N-acytl cysteine (NAC) were from Invitrogen; Gly-Phe β-naphthylamide (GPN) from Santa Cruz Biotechnology; Magic Red™-(RR)₂Cathepsin B assay kit from Marker Gene Technologies, Inc.

Antibodies.

The following antibodies were used for immunoblot: GAPDH (cat. 5174, 1:1,000), SQSTM1/p62 (cat. 5114, 1:1,000), TFEB (cat. 4240, 1:1,000), LAMIN A/C (cat. 2023, 1:1000), AMPKα-pThr172 (cat. 2535, 1:1,000), total AMPKα (cat. 5831, 1:1,000), ACC-pS79 (cat. 1:1000), total ACC (cat. 3676, 1:1,000), S6-pS235/236 (cat. 4858, 1:1,000), total S6 (cat. 2217, 1:1,000), Na+, K+-ATPase α1 (cat. 3010, 1:1,000), p70-S6K-pThr389 (cat. 9234, 1:1,000), total p70-S6K (cat. 2708, 1:1,000), TSC2-pThr1462 (cat. 3611, 1:1,000), total TSC2 (cat. 3635, 1:1,000), AKT-pThr308 (cat. 2965, 1:1,000), pan AKT (cat. 4691, 1:1,000), IP3R1 (cat. 3763, 1:1,000) and TFE3 (cat. 14779, 1:1000) were from Cell Signaling Technology and PPP3CB (cat. ab191374, 1:1000) was from Abcam; the following antibody was used for immunofluorescence: TFEB (cat. sc-48784, 1:100) from Santa Cruz Biotechnology and NFAT1 (cat. 5861, 1:100) and LAMP1 (cat. 9091, 1:200) from Cell Signaling Technology; the following antibody was used for immunohistochemistry: p62/SQSTM1 (cat. ab91526, 1:200) from Abcam.

Cell Culture and siRNA Transfection.

HeLa cells and MEFs were purchased from ATCC and were cultured in DMEM (Invitrogen) with 10% FBS and 1% antibiotics (Invitrogen). Earle's Balanced Salt Solution (EBSS, 10×, Sigma) was diluted to 1× with Milli-Q water supplemented with 2.2 g L-1 sodium bicarbonate (Sigma). HeLa cells that stably express GFP-TFEB and GFP-LC3, and p53^{-/-} and p53^{-/-} and TSC2^{-/-} MEFs and HepG2 cells were generous gifts from Dr. Shawn Ferguson (Yale University, USA), Dr. Beth Levine (UT Southwestern Medical Center, USA), Dr. James Brugarolas and Dr. Yihong Wan (UT Southwestern Medical Center, USA), and were cultured under the same conditions as described above. In the GFP-LC3 chemical screen, 2 mM NH₄C (Sigma) was supplemented in DMEM. All cell-based studies were performed with 25 mM HEPES buffer in a humidified chamber with 5% CO2. All cell lines have been tested for mycoplasma contamination using a MycoFluor™ Mycoplasma Detection Kit (Invitrogen). RNAi was performed by transfecting siRNA oligos (Dharmacon, Inc.) via reverse transfection using RNAiMax (Life Technologies) according to the manufacturer's instructions. A pool of four siRNA oligos targeting each gene was used to dilute off-target effects. Pools of four siRNAs targeting LONRF1 were used for transfection controls.

Synthesis of PEO-b-PR Block Copolymers.

In a typical procedure using PEO-b-PDBA80 (UPS_{5.3}) as an example, DBA-MA (1.92 g, 8 mmol), PMDETA (21 μl, 0.1 mmol) and MeO-PEO114-Br (0.5 g, 0.1 mmol) were charged into a polymerization tube. The monomer and initiator were dissolved in a mixture of 2-propanol (2 ml) and dimethylformamide (DMF) (2 ml). Three cycles of freeze-pump-thaw were performed to remove the oxygen, then CuBr (14 mg, 0.1 mmol) was added into the tube protected by nitrogen, and the tube was sealed in vacuo. After 8 h polymerization at 40° C., the reaction mixture was diluted in 10 ml tetrahydrofuran (THF), and the mixture was passed through a neutral Al2O3column to remove the catalyst. The organic solvent was removed by rotovap. The residue was dialyzed in distilled water and lyophilized to obtain a white powder.

Preparation of UPS Nanoparticle Solutions.

In a typical procedure, 10 mg UPS polymer was dissolved in 500 μL THF (UPS_{4.4}) or methanol (always-on/OFF-ON UPS_{5.3}). For always-on/OFF-ON UPS_{5.3} nanoprobes, BODIPY-conjugated polymer and Cy3.5-conjugated polymer was mixed with a 3:2 weight ratio. The solution was added to 10 mL Milli-Q water drop by drop. Four to five filtrations through a micro-ultrafiltration system (<100 kDa, Amicon Ultra filter units, Millipore) were used to remove the organic solvent. The aqueous solution of UPS nanoparticles was sterilized with a 0.22 μm filter unit (Millex-GP syringe filter unit, Millipore).

High-Throughput GFP-LC3 Chemical Screen.

GFP-LC3 HeLa cells were seeded in 384-well plates. UPS_{4.4} nanobuffer solutions were added the following day, and compounds (2.5 μM) were added for 4 hr on the third day. UPS_{4.4}-only cells were used as positive controls and wild-type HeLa cells were used as negative controls. Cells were then fixed with 4% formaldehyde, stained with 0.01% Hoechst 33324, and then sealed and read on with PHERAstar FS HTS microplate reader (BMG LABTECH). A saline-only plate was used to control background signals. Genedata Screener software (GeneData, Inc. Basel, Switzerland) was used to process and analyze the results. For each plate, the raw fluorescence GFP values were normalized with corresponding Hoechst signals after background-correction for all wells. The converted data was then normalized using Equation 1. Normalized well values were then corrected for position artifacts based on GeneData proprietary pattern detection algorithms. Finally, robust Z scores were calculated using Equation 2.

Normalized Data=Converted Dataₛₐₘₚₗₑₛ−Median of Converted-Data_{DMSO}/[Median of Converted Data_{positive control}−Median of Converted Data_{DMSO}]×100 (1)

Robust Z score=Converted Dataₛₐₘₚₗₑₛ−Median of Converted Data_{all sample}/Converted Robust Stand Deviation_{all sample} (2)

For the primary screen, each compound was tested as N=1, and primary hits were selected with robust Z scores less than −3. For the validation screen, the primary hits were assayed in triplicate. For each compound, the normalized activity values were condensed to a single value (condensed activity score) using the “Robust Condensing” method in Genedata Screener. The condensed activity is the most representative single value of the triplicates. Thirty compounds with lowest condensed activity values and robust Z score values were selected as the final hits.

High-Content GFP-TFEB Chemical Screen.

GFP-TFEB HeLa cells were seeded in 384-well plates. Compounds were added for 4 hr the following day. Bafilomycin A1 (250 nM) treated cells were used as positive controls and DMSO-treated cells as negative controls. Cells were fixed with 4% formaldehyde, stained with 0.01% Hoechst 33324, and then imaged on a GE IN Cell 6000 automated microscope with a OX objective. Images were collected using 405 nm and 488 nm laser lines with DAPI and FITC emission filters. Images were analyzed using the GE IN Cell Analyzer Workstation software. Briefly, nuclei were segmented using the Hoechst channel and the cytoplasm was segmented using the GFP channel. For each cell, the mean GFP intensity in each compartment was measured and used to calculate the nuclear to cytoplasmic (N/C) TFEB-GFP ratio. The same method as mentioned above was used to generate the top 30 compounds with highest condensed activity values and robust Z score values.

Isolation and Purification of Ikarugamycin.

SW201073 was extracted from marine-derived bacterium strain SNB-040 isolated from a sediment sample collected from Sweetings Cay, Bahamas. Bacterial spores were collected via a stepwise centrifugation as follows: 2 g of sediment was dried over 24 hr in an incubator at 35° C. and the resulting sediment added to 10 mL sH₂O containing 0.05% Tween 20. After vigorous vortex for 10 min, the sediment was centrifuged at 18000 rpm for 25 min (4° C.) and the resulting spore pellet collected. The resuspended spore pellet (4 mL sH₂O) was plated on an acidified JMA media, giving rise to individual colonies of SNB-040 after 2 weeks. Analysis of the 16S rRNA sequence of SNB-040 revealed 99% identity to Streptomyces phaeochromigenes. Bacterium SNB-040 was cultured in 20×2.8 L Fernbach flasks each containing 1 L of seawater-based medium (10 g starch, 4 g yeast extract, 2 g peptone, 1 g CaCO₃, 40 mg Fe₂(SO₄)3.4H₂O, 100 mg KBr) and shaken at 200 rpm at 27° C. After seven days of cultivation, sterilized XAD-7-HP resin (20 g L-1) was added to absorb the organic products, and the culture and resin were shaken at 200 rpm for 2 h. The resin was filtered through cheesecloth, washed with deionized water, and eluted with acetone. The acetone-soluble fraction was dried in vacuo to yield 4.5 g of extract. Crude extract of SNB-040 was fractionated using reverse phase flash column chromatography (C18) with a stepwise gradient (20%-100%) MeOH/H2O. Fractions were analyzed by LC-MS using an analytical C18 column and gradient from 10-100% acetonitrile/water (0.1% formic acid) over 17 minutes (0.7 mL min-1), followed by 100% acetonitrile for 5 minutes. Ikarugamycin elutes at 21 minutes on this LC-MS method. Fractions containing ikarugamycin were combined, dried and purified using reverse phase HPLC (phenyl-hexyl column, Phenomenex Luna, 250 mm×10.0 mm, 5 μm) at 80% acetonitrile/water (0.1% formic acid) and ikarugamycin tR=12.5 minutes with a strong UV absorbance at 254 nm. Ultimately a white amorphous solid (ikarugamycin) with a λmax absorption of 250 nm and 325 nm with m/z [M+H] of 479.2 was purified. 1H NMR (600 MHz, DMSO-d6) δ: 7.69 (dd, J=5.7 Hz, 1H), 7.46 (d, J=15.3 Hz, 1H), 6.45 (br s, 1H), 6.05 (dd, J1=15.3 Hz, J2=9.9 Hz, 1H), 5.94 (dt, J1=15.5 Hz, J2=14.4 Hz, 1H), 5.86 (d, J=9.6 Hz, 1H), 5.82 (dd, J1=14.4 Hz, J2=2.0 Hz, 1H), 5.72 (dd, J1=9.6 Hz, J2=2.0 Hz, 1H), 3.38 (m, 1H), 3.30 (m, 1H), 3.28 (m, 1H), 2.49 (m, 1H), 2.39 (m, 1H), 2.25 (m, 2H), 2.10-2.09 (m, 2H), 2.02 (m, 1H), 1.99 (m, 1H), 1.71 (m, 1H), 1.63 (m, 1H), 1.53 (m, 1H), 1.45 (m, 1H), 1.33-1.31 (m, 3H), 1.26 (m, 1H), 1.16-1.09 (m, 3H), 0.91 (dd, J1=8.3 Hz, J2=7.2 Hz, 3H), 0.86 (d, J=7.2 Hz, 2H), 0.66 (m, 1H). 13C NMR (100 MHz, DMSO-d6) δ: 195.3, 181.3, 176.6, 165.6, 140.1, 137.8, 132.0, 130.2, 129.1, 125.0, 101.9, 58.4, 48.6, 48.3, 47.3, 46.7, 46.5, 42.1, 41.1, 38.3, 37.9, 36.9, 33.7, 27.2, 24.8, 21.6, 21.1, 17.7, 13.1.

Dose-Response Assays.

Wild-type, GFP-LC3 and GFP-TFEB HeLa cells were seeded in 96-well plates and treated with half log dilutions of compounds in triplicates. Treatment, data acquisition and analysis was identical to that described for the HTS chemical screens. Cathepsin B activity was measured using the Magic Red™-(RR)₂ Cathepsin B assay kit following a 4 hr treatment with compounds at indicated doses. Raw data was background-corrected, log-transformed and fit with the dose-response function with Graphpad Prism (v6.0) software.

Confocal Imaging.

All confocal imaging was performed on a Zeiss LSM 700 laser scanning confocal microscope using a 40×/1.3DIC objective. Cells were plated on 4- or 8-well Nunc™ Lab-Tek™ II Chambered Coverglass (Thermo Scientific) and allowed to grow for 24 h. After treatment, cells were fixed in 4% formaldehyde and imaged at excitation wavelengths of 488 nm (GFP, LysoSensor Green or BODIPY) and 560 nm (Cy3.5). ImageJ software (NIH) was used to process and analyze the images.

Endosomal Maturation Rates.

HeLa cells were plated on 4- or 8-well Nunc™ Lab-Tek™ II Chambered Coverglass and allowed to grow for 24 h. After 4 hr treatment with compounds or DMSO, cells were incubated with always-ON/OFF-ON UPS_{5.3} nanoprobes for 5 min in serum-free medium, then washed three times with PBS before imaging. The FIOFF-ON (BODIPY)/FIAlways-ON (Cy3.5) ratio was quantitated with ImageJ. For each cell, a region of interest was defined as the punctae in cytosol that emitted fluorescent signals from both BODIPY and Cy3.5 channels. Fluorescent intensity ratio was calculated for each intracellular punctate as R=(F1−B1)/(F2−B2) where F1 and F2 are the fluorescence intensities from BODIPY and Cy3.5 channels respectively, and B1 and B2 are the corresponding background values determined from a region on the same images that was near the punctae in the cytosol. All the ratios of each nanoprobe were normalized to their end time-point ratio, and the curves were fit with Graphpad Prism (v6.0) software.

Fura-2 Ca²⁺ Imaging.

HeLa cells treated with compounds or DMSO for 4 hr were loaded with Fura-2-AM (3 μM) in cell culture medium for 60 min at 37° C. Cells were washed twice then incubated in fresh medium for 30 min to allow complete de-esterification of intracellular AM esters. Imaging was performed at 37° C. with 5% CO₂ on an epifluorescent microscope (Deltavision, Applied Precision) equipped with a digital monochrome Coolsnap HQ2 camera (Roper Scientific, Tucson, Ariz.). Fluorescence images were collected using SoftWoRx v3.4.5 (Universal Imaging, Downingtown, Pa.). Data were recorded at excitation/emission wavelengths of 340/510 nm (Fura-340 filter) and 387/510 nm (Fura-380 filter). The single band pass excitation filter for Fura-340 and Fura-380 is 26 nm and 11 nm, respectively, and the band pass of emission filters for Fura-340 and Fura-380 is 84 nm. Intracellular calibration of Fura-2 was accomplished by manipulating the Ca²⁺ levels inside cells using the ionphore ionomycin (20 μM, Sigma-Aldrich) and by incubating cells in buffers with various Ca²⁺ concentrations (calcium calibration buffer kit—Invitrogen). Intracellular fluorescence ratios were determined using ImageJ software. Images were background-corrected by subtracting the mean pixel values of a cell-free region near the region of interest. Fluorescent intensity ratio R=F340/F380, and Ca²⁺ concentration can be calculated from Equation 3, where Kd can be obtained from intracellular calibration:

[Ca²⁺]K_{d}×F_{380,min}/F_{380,max}×(R−Rₘᵢₙ)/(Rₘₐₓ−R) (3)

GFP-TFEB Nuclear-Translocation.

GFP-TFEB HeLa cells treated with compounds or DMSO were fixed and stained with Hoechst. Images from at least 3 different fields per sample were acquired using a 40× objective on a Zeiss confocal microscope and analyzed with ImageJ. 20-30 cells were evaluated from each image for each sample, and 3 independent experiments were performed to generate the graphed values.

RNA Extraction and qRT-PCR.

Total RNA was isolated from MEFs or mouse tissues using RNeasy minipreps (QIAGEN). Complementary DNA (cDNA) was synthesized with the High capacity RNA-to-cDNA kit (Applied biosystems), and qRT-PCR was performed using TaqMan® Gene Expression Assays (Applied biosystems) for the indicated genes on the LightCycler System (Roche Applied Science). Gapdh was used to normalize RNA input. The mouse probes used in this study were: Tfeb (Mm00448968_m1), Ctsa (Mm00447197_m1), Mcoln 1 (Mm00522550_m1), Ppara (Mm00440939_m1), Ppargc1a (Mm01208835_m1), Fgf21 (Mm00840165_g1) and Gapdh (Mm99999915_g1), and the human probes used were TFEB (Hs00292981_m1), CTSA (Hs00264902_m1), MCOLN1 (Hs01100653_m1), PPARGC1A (Hs00173304_m1), PPARA (Hs00947536_m1), UVRAG (Hs01075434_m1), GAPDH (Hs02758991_g1) and p62/SQSTM1 (Hs01061917_g1). All probes were from Thermo Fisher Scientific Inc.

RNA Interference (RNAi).

Transfection of siRNA duplexes was used to silence indicated genes. In brief, cells grown in six-well plates were transfected with Lipofectamine® RNAiMAX transfection reagent (Thermo Fisher Scientific) and 100 nM siRNA duplexes targeted against Na⁺—K⁺-ATPase α1 subunit (MQ-006111-02), PTPMT (MQ-029988-02), PPP3CB (MQ-009704-01), MCOLN1 (MQ-006281-00), TFEB (MQ-009798-02), and TFE3 (MQ-009363-03, Dharmacon). Treated cells were analyzed 48-72 hours after transfection.

Nanoparticle Formulation.

AD or IKA (1 mg) together with PEG-PLA polymer (9 mg) were first dissolved in 1 mL methanol. The solution was added drop-wise to 10 mL Milli-Q water. Four to five filtrations through a micro-ultrafiltration system (<100 kDa, Amicon Ultra filter units, Millipore) were used to remove the organic solvent and unencapsulated free drugs. The aqueous solution of UPS nanoparticles was sterilized with a 0.22 μm filter unit (Millex-GP syringe filter unit, Millipore). Micelle solutions were then lyophilized and the resulting freeze-dried powder was weighed, dissolved in a mixture of methanol and deionized water (v/v=9/1), and analyzed using a Shimadzu UV-1800 UV-Vis spectrophotometer (k=240 nm, extinction coefficient=2.0×104 M-1 cm-1) to calculate the total amount of micelle encapsulated drug. Nanoparticles were also characterized by dynamic light scattering (DLS) to evaluate particle size.

Mouse Model for In Vivo Compound Delivery.

All mouse experiments were approved and carried out following the ethical guidelines established by the Institutional Animal Care and Use Committee at UT Southwestern Medical Center. The investigators were not blinded to allocation during experiments and outcome assessment. Four to six weeks old male C57BL/6J mice were randomly divided into groups fed a regular diet (Harlan Teklad) or a high-fat diet (HFD) containing 60% fat (Research Diets). After one month, the HFD mice were grouped so that the average body weights of mice in each group were similar. The grouped mice were orally administered DG (2.5 mg kg-1) or its solvent 50% HPβCD solution, or intravenously injected with AD (1 mg kg-1) or IKA (0.5 mg kg-1) encapsulated in the PEG-PLA nanoparticles or empty nanoparticles, three times a week for three weeks. Body weight and food intake were measured twice a week before treatment and every day after treatment in the middle of the light period. The cage tops containing food pellets were weighed, as well as the spilled food in the bottom of the cage. The food intake was corrected for spillage.

In Vitro Compound Release from PEG-PLA Micelle.

AD and IKA release from PEG5000-PLA5000 micelle was measured using a dialysis method. In a typical procedure, AD or IKA micelle solution (0.5 mL, 10 mg mL-1) was added to the upper chamber of a 15 mL mini dialysis tube (3.5 k molecular weight cutoff, Fisher Scientific Inc.) with 1×PBS with 1% Tween 80 (Sigma-Aldrich). At different time points, 1 mL solution was removed from the tube and replaced with 1 mL 1×PBS with 1% Tween 80. The released AD or IKA was determined by measuring the UV-Vis absorbance of the obtained solution based on the standard curves of AD and IKA. Percentage of compound release was plotted as a function of time to show the release kinetics.

Body Composition Analysis.

At the end of the treatment, the body composition of each mouse was analyzed by EchoMRI (Echo Medical Systems LLC) according to the manufacturer's instructions.

Glucose and Insulin Tolerance Tests.

For glucose tolerance tests, the mice were orally administered 1 mg g-1 glucose (Sigma-Aldrich) after a 4 hr fast. For insulin tolerance tests, the mice were intraperitoneally injected with 0.75 milliunit g-1 insulin (Humulin R, Eli Lily) after a 4 hr fast. Blood was drawn from tail veins at indicated time points after injection. Experiments were performed during light period. Serum glucose levels were analyzed using commercial glucose reagents (Sigma).

Serum Chemistry Analysis.

At the end of the treatment, blood was collected from the orbital plexus under anesthesia. Serum was frozen in aliquots and stored at −20° C. for further analysis. Specific enzyme kits were used to detect serum levels of triglyceride (Fisher Scientific), cholesterol (Fisher Scientific) and glucose (Sigma-Aldrich).

Histology.

Livers and other organs were dissected and embedded in OCT. Cryostat sections were cut at 10 μm. The sections were stored at −80° C. and subjected to haematoxylin/eosin and oil red O staining following standard protocol. The immunohistochemistry (IHC) staining of p62 was performed following the protocol of Cell Signaling Technology. The primary antibody was from Abcam, and the SignalStain® boost IHC detection reagent and DAB substrate kit were from Cell Signaling Technology. All the sections were imaged using a NanoZoomer 2.0-HT Digital slide scanner (Hamamatzu) and processed using NDP viewer software.

Cytotoxicity.

HeLa cells were plated in a 96-well plate with a white wall and a clear bottom. After 24 hours, cells were treated with various doses of DG, AD and IKA for 4 hours. Cells were then washed with PBS 3 times, and viability was determined immediately or after 72 hours using CellTiter-Glo® Luminescent Cell Viability Assay (Promega).

HLH-30::GFP Nuclear Localization Assay.

Adult TX1941 dal-1(dt2300); sqIs19 [hlh-30p::hlh-30::GFP rol-6(+)] worms were placed on NGM plates with either a test chemical or 5% DMSO. GFP was scored at various intervals on live worms without mounting using a Zeiss Axio Zoom. V16 fluorescence dissecting microscope equipped with Axiocam 503. No difference was observed between 2 hour and overnight treatment.

C. elegans Lifespan Analysis.

The C. elegans mutant strain, fem-1(hc17ts) IV; dal-1(dt2300), obtained from the Caenorhabditis Genetics Center at the University of Minnesota, was cultured on Nematode Growth Medium (NGM) plates seeded with the E. coli strain OP50 and consistently maintained at 15° C. For lifespan assays, a total of 12 age-synchronized nematode adults were transferred to eight replicate NGM plates and grown at 25° C. to ensure sterility. Age synchronization was achieved through standard hypochlorite treatments. Eggs were placed on NGM plates supplemented with Streptomycin (100 μg ml-1) and seeded with the E. coli strain, OP50. For compound testing, 200 μl of 10 μM Ikarugamycin in 5% DMSO was spotted directly onto OP50 seeded NGM plates. Control plates were prepared by spotting 200 μl of 5% DMSO directly onto OP50 seeded NGM plates. For the first 10 days of adulthood, C. elegans were scored once a day as dead or alive by touch stimulation with a platinum wire. After day 10, animals were scored every other day. Nematodes which crawling off the agar plates were censored from subsequent lifespan analysis. Kaplan-Meier statistical analysis was performed using Prism 7 software. Lifespan experiments were done on two separate occasions.

Statistics.

Sample sizes and reproducibility for each figure are denoted in the figure legends. Data were presented as the mean±s.d unless specified. Analysis of variance (ANOVA) approaches were used for comparisons among experimental groups that met the normality distribution assumption. If not, the data was log-transformed or a non-parametric t-test was used. One-way ANOVA and two-way ANOVA were used for comparison within groups with single or two variables.

### Example 2—A UPS-Enabled High-Throughput Screen for TFEB Agonists

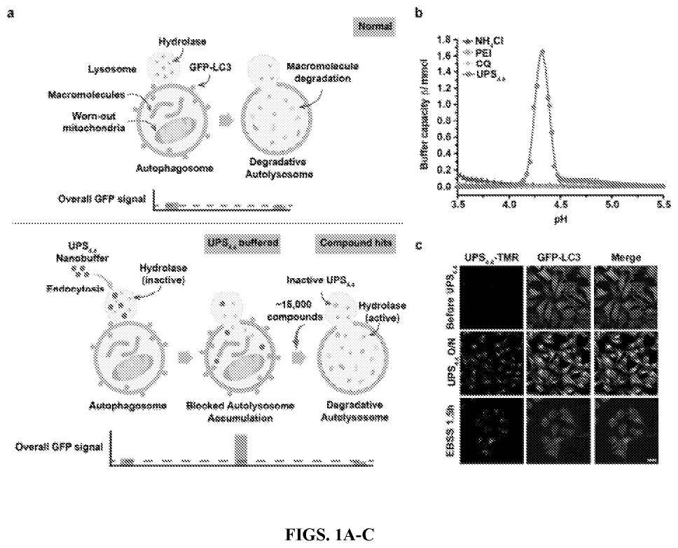

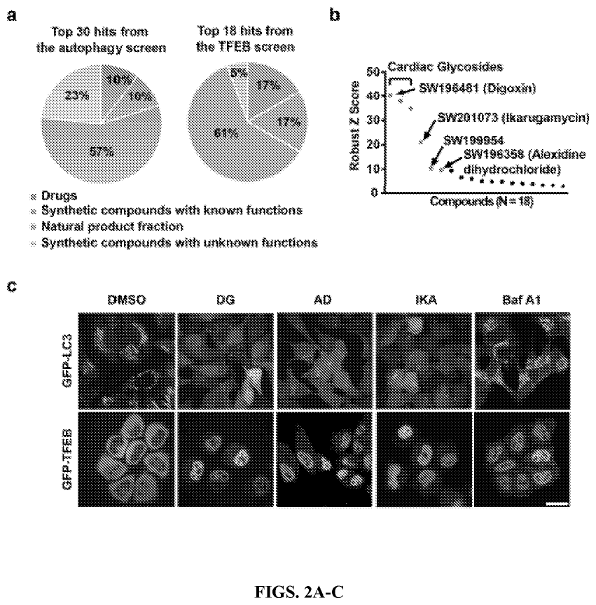

A key functional consequence of TFEB activation is enhanced clearance of deleterious macromolecules and organelles through autophagic and lysosomal degradation. In order to identify new chemical probes that promote TFEB activity, a quantitative high-throughput cell-based assay for agents that promote maturation of autophagosomes to degradative autolysosomes (FIG. A) was designed. This was enabled by a fine-scale UPS nanobuffer library²¹, wherein each micelle nanoparticle is composed of ˜800 copolymer chains with a total of about 60,000 ionizable tertiary amine groups²³. At specific transition pH, each micelle undergoes a phase transition, or de-micellization, which renders a strong buffering capacity within 0.3-pH range. This pH cooperative buffer effect was implemented to clamp the luminal pH of endocytic organelles at distinct maturation stages²². Among the UPS nanoprobes, UPS_{4.4} arrests lysosomal acidification at pH ˜4.4 (FIG. 1B and SFIG. TA), thereby inhibiting lysosomal/autolysosomal hydrolysis of macromolecules without inhibiting the regulation of mammalian target of rapamycin complex 1 (mTORC) on lysosomes²². Turnover of microtubule-associated protein A/1B light chain 3 (LC3), the ortholog of yeast autophagy-related protein 8 (ATG8), was selected as a quantitative measure of lysosome maturation. LC3-II (lipid-modified form of LC3) coats the double membrane structures that encapsulate material that is delivered to autolysosomes and is itself degraded within those compartments²⁴. GFP-LC3 fusion proteins were employed as live-cell markers for monitoring autophagic flux. UPS_{4.4} exposure was sufficient to induce accumulation of cytoplasmic GFP-LC3 puncta (FIG. 1C and SFIG. 1B) that colocalized with fluorescently labeled UPS_{4.4} nanoprobes as well as lysosomal marker LAMP1 (UPS_{4.4}-TMR, SFIGS. 1C-D). Moreover, nutrient restriction was sufficient to promote vacuolar ATPase-dependent clearance of these puncta within 90 minutes, with consequent reduction of GFP fluorescence intensity (FIG. 1C and SFIGS. 1E-F), resulting in an almost binary ON/OFF signal that can be accurately measured by a microplate reader in a high throughput screen setting. Blocking autolysosomal functions by UPS_{4.4} resulted in an increase in GFP-LC3 puncta accumulation and fluorescence intensity (6-fold) over starvation-induced autophagic degradation. This is in contrast to a maximal 1.5-fold fed versus starvation signal in the absence of UPS_{4.4} (SFIGS. 1G-H). The clearance of UPS_{4.4}-induced accumulation of GFP-LC3 indicated the presence of a dynamic cell biological system, which promotes induction of autophagosome maturation in response to nutrient starvation and is amenable to chemical interrogation. This system was leveraged to evaluate 15,000 chemical entities for cellular activity that mimics nutrient starvation (SFIGS. 2A-D). Thirty (out of 80) primary hits were confirmed by independent analyses (FIG. 2A and SFIGS. 2B and 2E). Hits were evaluated for effects on TFEB activity under nutrient replete culture conditions. Transcriptional competence of TFEB is modulated by physical compartmentalization in the cytoplasm (off-state) versus the nucleus (on-state). Chemically induced TFEB nuclear translocation was monitored using the fluorescent intensity ratio of nuclear versus cytoplasmic GFP-TFEB (SFIGS. 2F-H). The quantitative robustness of these assays was indicated by a low coefficient of variance (% CV) and a high Z-factor calculated for the neutral control condition (SFIG. 2I). The top scoring hits included 3 cardiac glycosides (digoxin, proscillaridin A and digoxigenin), two natural-product fractions (SW201073 and SW199954), and the synthetic small molecule alexidine dihydrochloride (FIGS. 2A-B and SFIG. 2J). Structure determination revealed the bioactive component of SW201073 is identical to ikarugamycin (SFIGS. 2K-L), a macrocyclic antibiotic first isolated from Streptomyces phaeochromogenes²⁵. Mechanism of action studies were further pursued with digoxin (DG), alexidine dihydrochloride (AD) and ikarugamycin (IKA). Consistent with the primary screen results, all three compounds promoted autophagic flux and activated TFEB in a dose-dependent manner as indicated by clearance of UPS_{4.4}-dependent accumulation of GFP-LC3 puncta; increased autophagic flux (conversion of LC3-I to LC3-II); turnover of the long-lived autophagy adaptor protein p62/SQSTM1²⁶; and translocation of GFP-TFEB from cytosol to nucleus (FIG. 2C and SFIGS. 2M-R). The activity of DG was consistent with isolation of this compound as a hit in a Prestwick-library-focused screen for compounds that promote bulk autophagy²⁷. The EC₅₀ of these compounds for promotion of p62/SQSTM1 clearance was generally higher than corresponding TFEB nuclear accumulation EC_{50S} consistent with time and signal delta between TFEB activation and transcription-dependent autophagy induction (SFIGS. 2M, 2P and 2Q). Activation of TFEB induces the expression of numerous lysosomal and autophagic genes that promote lysosomal biogenesis and maturation^{9,11}. All three compounds induced the expression of TFEB target genes⁷ (SFIG. 2S), and accelerated cellular endosomal maturation rates as indicated by accumulation of acidified organelles (SFIG. 2T) and activation of cathepsin B (SFIG. 2U). Furthermore, siRNA-mediated TFEB depletion was sufficient to inhibit the capacity of the compounds to induce autophagic flux or activate TFEB target genes (SFIGS. 2V and 2X). Depletion of both TFEB and its homolog, TFE3, further hindered cellular responses to all three compounds (SFIGS. 2W and 2X).

### Example 3—Engagement of mTORC1 by TFEB Agonists

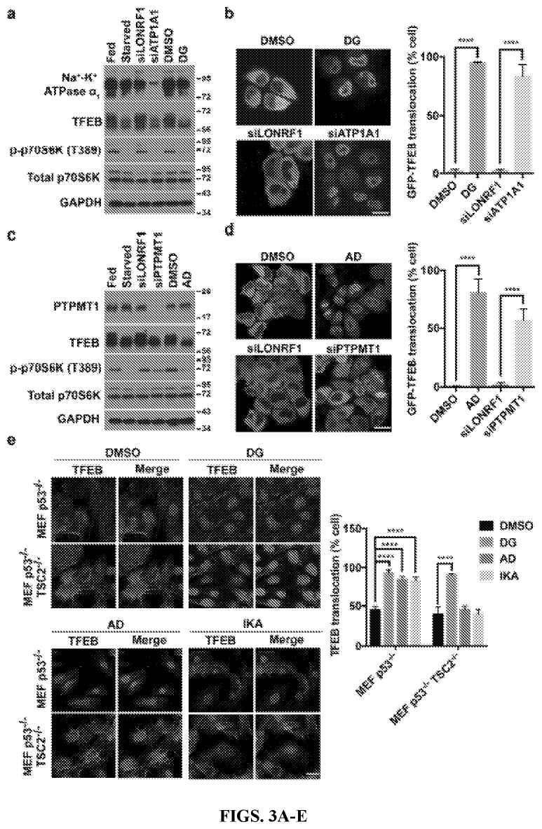

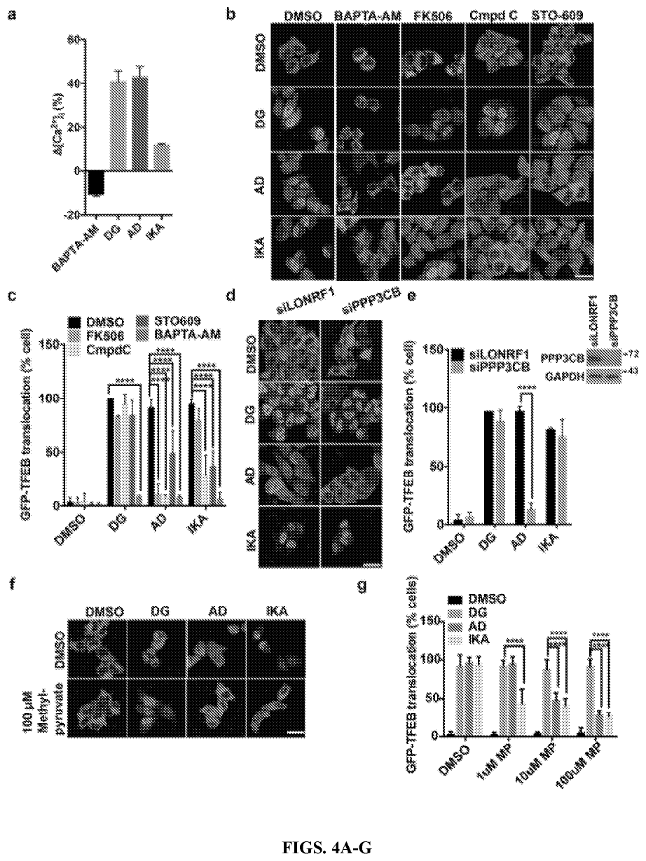

Drect molecular targets of DG and AD in cells are the Na⁺—K⁺ ATPase α1 subunit (encoded by ATP1A1)²⁸ and the protein tyrosine phosphatase mitochondrial 1 (PTPMT1)²⁹, respectively. Short interfering RNA (siRNA) mediated depletion of these targets recapitulated GFP-TFEB nuclear translocation in nutrient replete culture conditions, suggesting DG (FIGS. 3A-B) and AD (FIGS. 3C-D) engage TFEB through their reported cellular targets. The activity of TFEB is regulated by the kinase mTORC1 and the phosphatase calcineurin, where mTORC1 directly phosphorylates TFEB S142 and S211 to promote cytosolic sequestration via phospho-serine-dependent interaction with 14-3-3 proteins^{11,13,17} while calcineurin dephosphorylates TFEB and promotes its nuclear localization. To begin to parse how these targets engage TFEB, mTOR pathway activity was examined. Exposure to all three compounds as well as depletion of the known compound targets, Na⁺—K⁺ ATPase α1 subunit or PTPMT1, resulted in detectable dephosphorylation of the mTORC1 substrate p70 S6 kinase (p70S6K) under nutrient replete culture conditions, consistent with an inhibition of mTORC1 activity that would occur in response to nutrient starvation (FIGS. 3A and 3C). However, inhibition of mTORC1 and activation of TFEB by DG and proscillaridin A (PA, one of the cardiac glycoside hits) was independent of the nutrient responsive mTOR inhibitory component tuberous sclerosis 2 (TSC2) (FIG. 3E and SFIGS. 3A-C). By contrast, the impact of AD and IKA on mTORC1 pathway activity was TSC2-dependent (FIG. 3E and SFIG. 3D). This suggested distinct mechanisms of engagement of mTORC and TFEB by DG, AD and IKA.

### Example 4—Disparate Ca²⁺-Dependent Mechanisms Mediate AD, DG and IKA Induction of TFEB

A TFEB activation mechanism is calcium/calmodulin-dependent dephosphorylation of TFEB (S142) by the calcineurin protein phosphatase¹². DG, AD, or IKA exposure at ˜TFEB_{EC90} was sufficient to induce accumulation of cytosolic calcium as indicated by the quantification of Fura-2 imaging (FIG. 4A), and Ca²⁺ chelation by BAPTA-AM was sufficient to block TFEB activation and reverse mTORC1 inhibition by all 3 compounds (FIG. 4B and SFIG. 4B). Direct inhibition of calcineurin with FK506^{30,31}, cyclosporine A (CsA)^{30,31}, or RNAi-mediated depletion of the calcineurin catalytic subunit (PPP3CB) was also sufficient to block TFEB activation by AD, suggesting small molecule inhibition of PTPMT1 activates TFEB via mobilization of calcineurin catalytic activity (FIGS. 4B-E and SFIGS. 4B-C). DG induced TFEB activation was resistant to calcineurin perturbation at EC₉₀, but was inhibited to some extent at EC₅₀ (FIGS. 4B-E and SFIG. 4C). In contrast, IKA-induced TFEB activation was calcineurin independent (FIGS. 4B-E and SFIG. 4C). Consistent with that, AD, but not DG and IKA, induced dose-dependent nuclear translocation of nuclear factor of activated T cells (NFAT)³², which depend on the activity of calcineurin (SFIG. 4D).

Primary response to both elevated cytosolic Ca²⁺ and nutrient starvation is activation of AMP-activated protein kinase (AMPK)³³. AMPK mediates biological responses to caloric restriction through both mTOR-dependent and mTOR-independent mechanisms and is engaged directly by calcium/calmodulin-dependent protein kinase beta (CaMKKβ) upon elevation of cytosolic Ca²⁺. Chemical activation of AMPK either directly with 5-aminoimidazole-4-carboxamide ribonucleotide (AICAR)³⁴ or indirectly with metformin³⁵ was sufficient to induce TFEB nuclear accumulation (SFIG. 4E). Dorsomorphin (Compound C), an AMPK inhibitor and ST609, a CaMKKβinhibitor³⁶, both inhibited TFEB nuclear translocation induced by IKA and AD, but not DG. Furthermore, IKA and AD, but not DG, induced activating phosphorylation of AMPK and its downstream substrate acetyl-CoA carboxylase (ACC) in a dose-dependent manner (SFIGS. 4F-G). Finally, the effects of AD and IKA, but not DG, on TFEB can be reversed by addition of cell-permeable pyruvate, which increases intracellular ATP level and promotes inactivation of AMPK (FIGS. 4F-G). These observations indicate that distinct Ca²⁺-dependent mechanisms mediate AD, DG and IKA induction of TFEB. AD activity is calcinuerin- and AMPK-dependent, IKA is calcinuerin-independent but AMPK-dependent, and DG is relatively independent of both calcinuerin and AMPK.

### Example 5—Different Ca²⁺ Stores Contribute to TFEB Activation by DG and AD Versus IKA

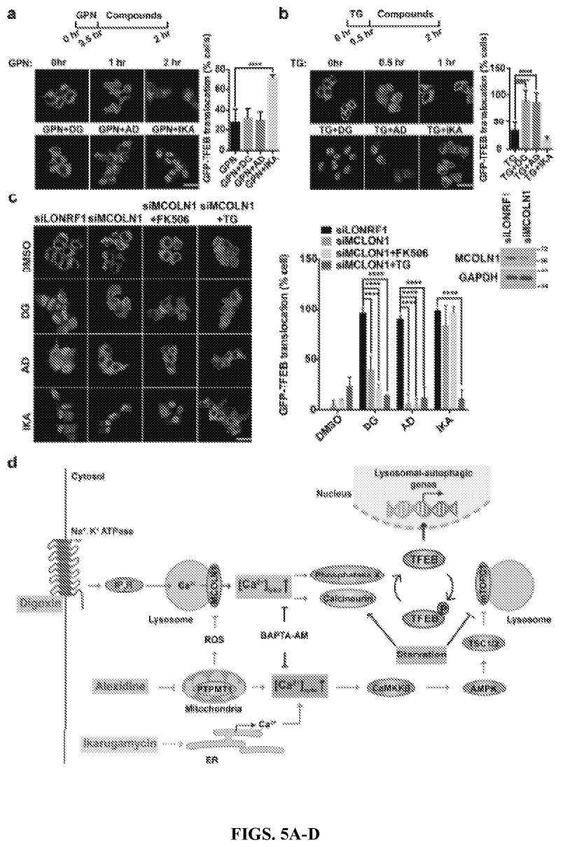

Lysosomes, mitochondria and endoplasmic reticuli (ER) are the major compartmentalized Ca²⁺ stores in cells³⁷. Glycyl-L-phenylalanine 2-napthylamide (GPN) is a lysosome-disrupting agent that is used to deplete lysosome-specific Ca²⁺ stores³⁸. A 30 min pretreatment of GPN was sufficient to disrupt TFEB nuclear translocation induced by DG and AD but had almost no effect on IKA-treated cells (FIG. 5A). In contrast, ER-specific depletion of Ca²⁺ by acute treatment with thapsigargin (TG), a specific inhibitor of ER Ca²⁺ ATPase SERCA pump³⁹, selectively decreased TFEB nuclear translocation induced by IKA (FIG. 5B). This suggests selective perturbation of lysosomal calcium pools by DG and AD versus ER calcium pools by IKA. RNAi-mediated depletion of the principal Ca²⁺ channel in lysosomes, mucolipin 1 (MCOLN1), suppressed TFEB nuclear translocation induced by DG and AD, but not IKA (FIG. 5C). MCOLN1 can be activated by reactive oxygen species (ROS) in the cells and thus activate TFEB in a lysosomal Ca²⁺/calcineurin-dependent manner⁴⁰. As AD impairs mitochondrial function through targeting PTPMT1, intracellular ROS levels were monitored as a potential explanation for the consequences of AD on TFEB activation. Consistent with this, tert-Butyl hydroperoxide (TBHP) and AD induced comparable ROS production and TFEB nuclear translocation. The effects of both compounds on these phenotypes were abolished by the membrane-permeable antioxidant n-acetyl-cysteine (NAC) (SFIGS. 5A-C). With respect to DG, the inventors noted that cardiac glycosides have been reported to promote the binding of Na⁺—K⁺ ATPase to IP3R, which can in turn induce downstream Ca²⁺ release through IP3R⁴¹ and refuel lysosomal Ca²⁺ store. A relatively specific IP3R inhibitor Xestosporin C (Xesto), as well as a siRNA-mediated depletion of type 1 IP3R (IP3R1), attenuated TFEB nuclear translocation induced by IKA, AD and DG in a time-dependent manner (SFIGS. 5D-I) consistent with the direct engagement of ER-versus lysosome-specific (indirectly through ER) Ca²⁺ by these compounds. Taken together, these observations indicate that distinct calcium stores mediate TFEB activation by DG and AD versus IKA (FIG. 5D).

### Example 6—TFEB Agonists Mitigate Metabolic Syndromes and Extend Lifespan In Vivo

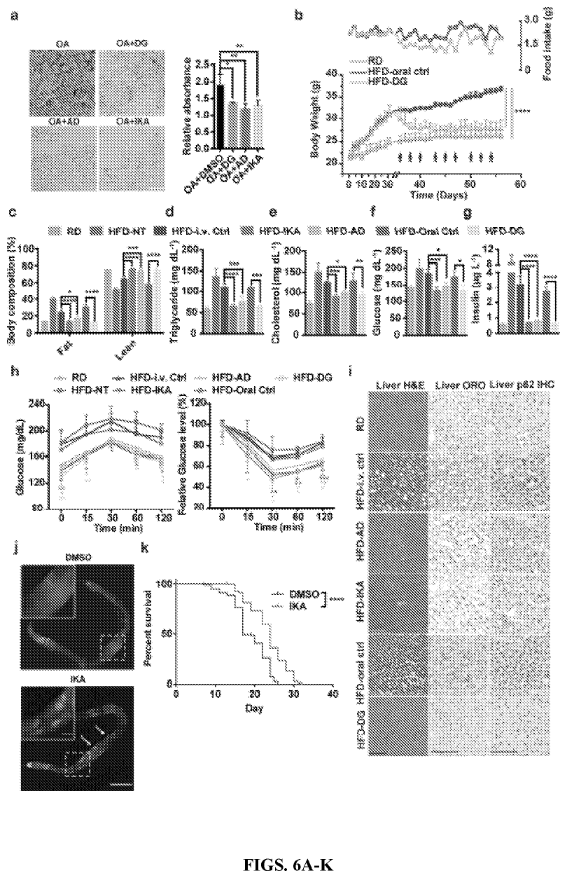

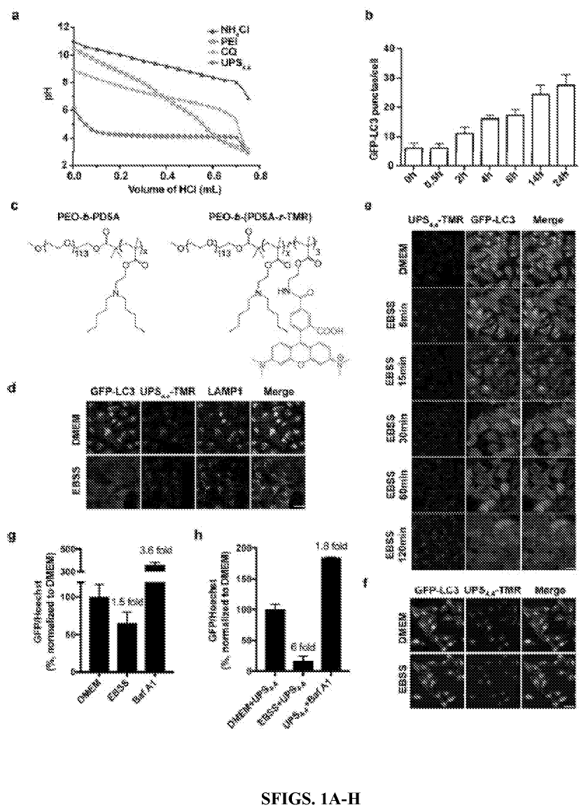

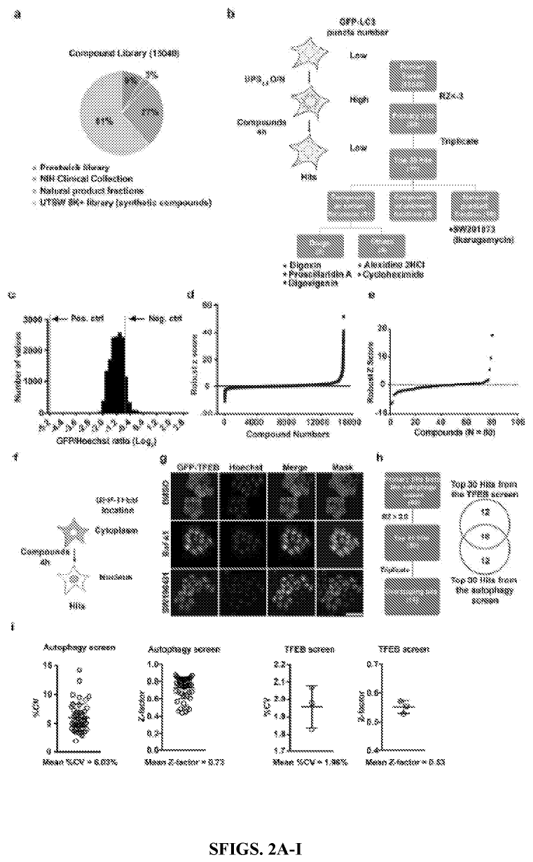

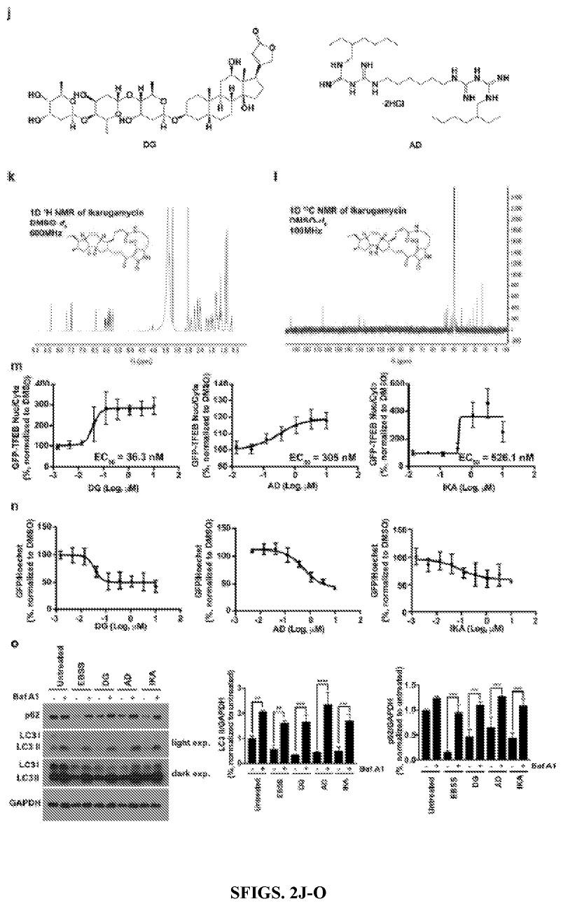

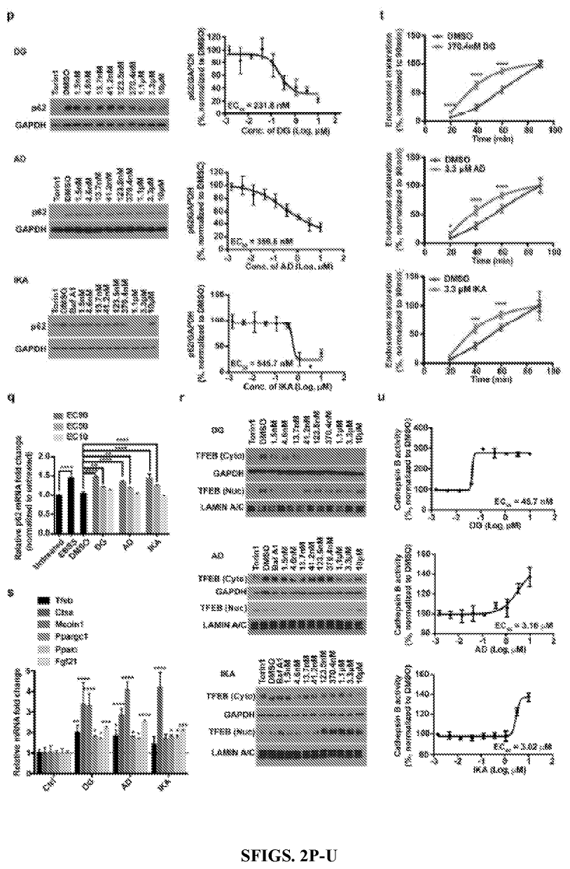

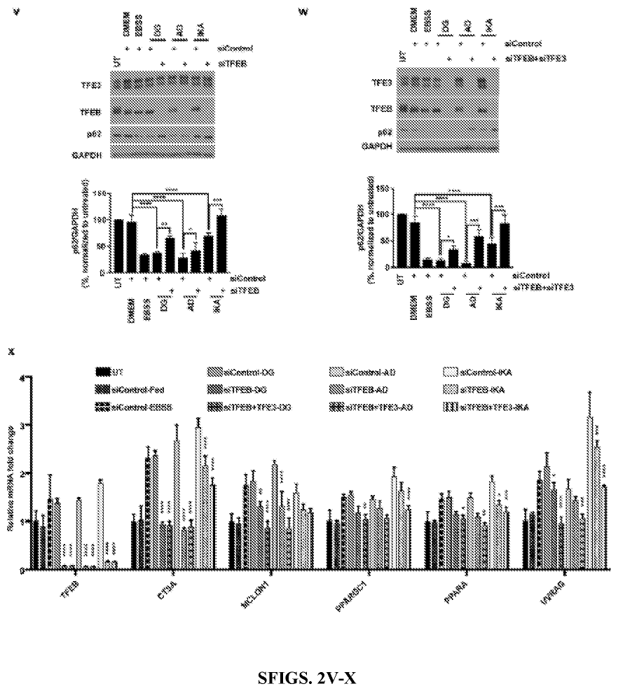

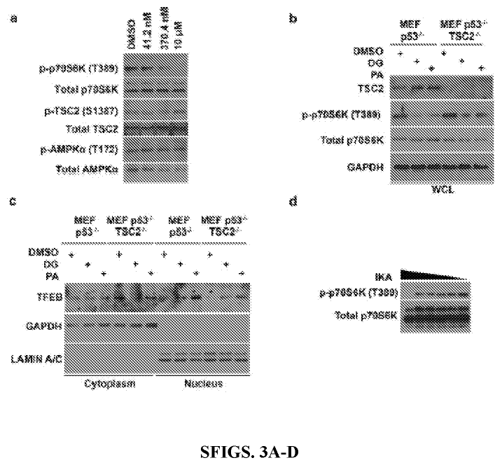

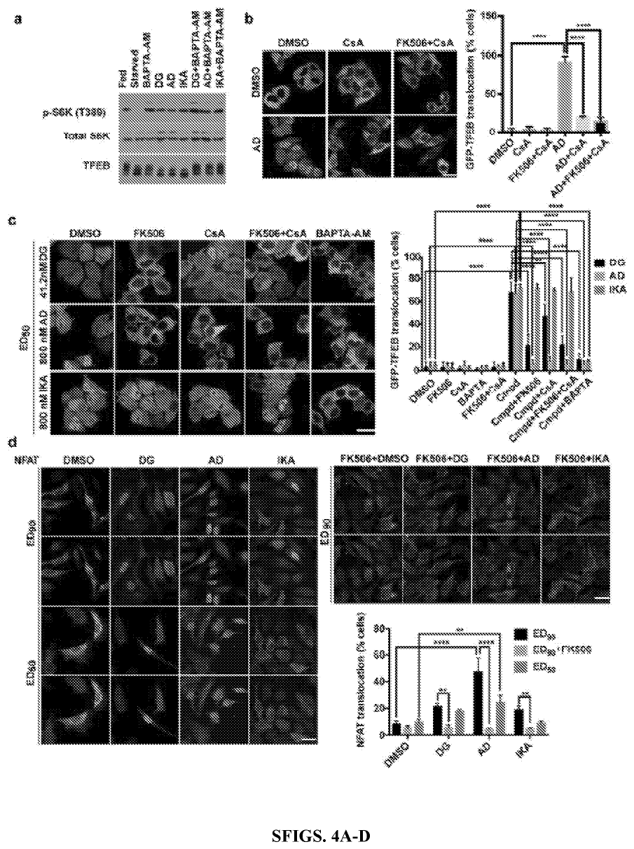

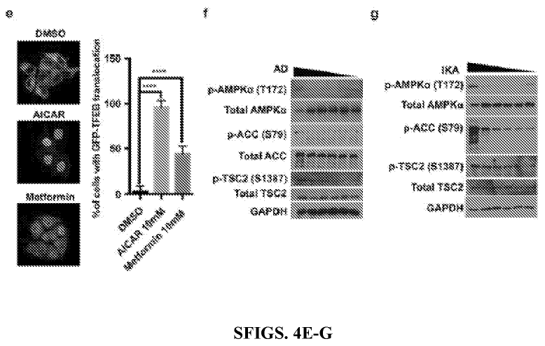

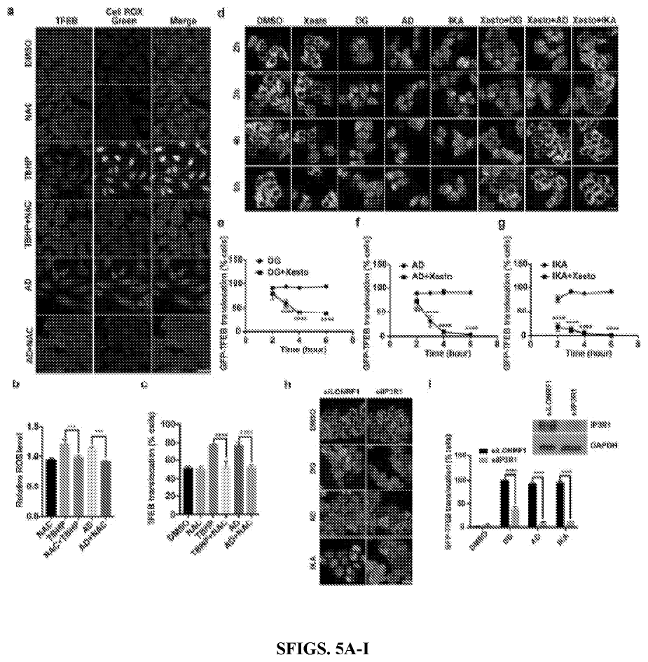

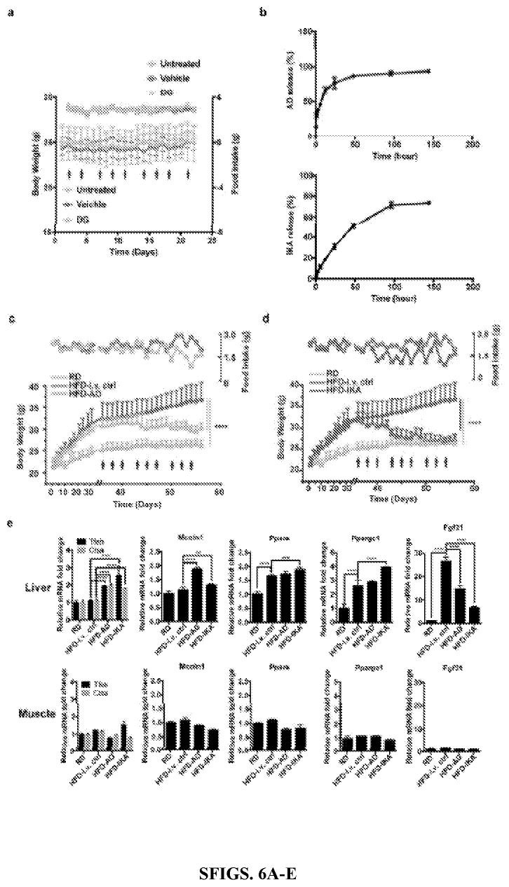

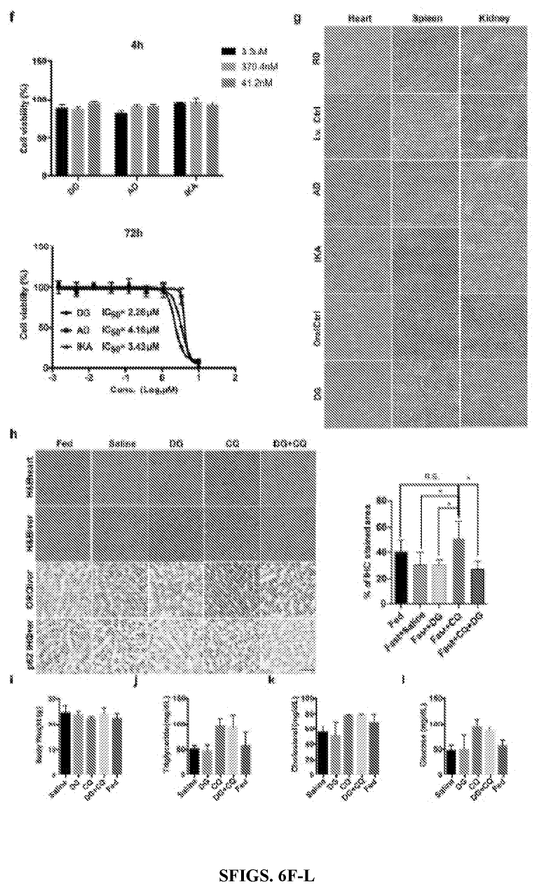

In animals, TFEB plays a key role in promoting lipid metabolism during starvation, at least in part through global transcriptional activation of peroxisome proliferator-activated receptor γ coactivator 1 α (Ppargc1α) and peroxisome proliferator-activated receptor α (Ppar1α)^{7,42}. Consistent with physiologically pertinent TFEB activation, DG, AD and IKA significantly ameliorated oleic acid-induced lipid accumulation in human hepatocytes (FIG. 6A). Oral administration of DG normalized body weight, body composition and circulating cholesterol, triglycerides, glucose and insulin levels in mice challenged with a high fat diet (FIGS. 6B-G). In contrast, DG did not alter the body weight of the lean mice (SFIG. 6A). For in vivo analysis of AD and IKA, compounds were encapsulated into biocompatible, biodegradable polyethylene glycol-b-poly (D, L-lactic acid) (PEG-PLA) nanoparticles that are liver-trophic^{43,44} (Supplementary Table 1). Controlled and sustained release of AD and IKA persisted for more than 2 days in an in vitro setting that mimics the in vivo environment (SFIG. 6B), supporting the 3-times-a-week treatment regimen. Like DG, significant normalization of body weight/composition and blood chemistry was observed upon high fat challenge relative to control groups (FIGS. 6C-G and SFIGS. 6C-D). Moreover, compound-treated mice also displayed improved glucose and insulin tolerance relative to control animals (FIG. 6H). Liver histology revealed amelioration of high fat diet-induced steatosis, which corresponded to upregulation of Ppargc1α, Ppar1α and Fgf21^{45,47} by DG, AD and IKA (FIG. 6I and SFIG. 6E). Compound treatment also reversed p62/SQSTM1 accumulation in hepatocytes, suggesting enhanced autophagic flux in these mice (FIG. 6I). No obvious toxicity to major organs was observed in any treatment group, nor was it observed in the in vitro experiments (SFIGS. 6F-G). An overnight fast in mice is sufficient to induce a transient increase in hepatic lipid accumulation as a consequence of adipose tissue lipolysis⁴⁸, a phenotype that is exacerbated by chloroquine (CQ) (SFIGS. 6H-I). This effect can be improved by co-administration of DG, consistent with an enhanced endolysosomal function engaged by TFEB in hepatocytes (SFIGS. 6J-L); an effect also consistent with DG-dependent reduction in p62/SQSTM1 accumulation (SFIG. 6H). These observations indicate that TFEB activation induced by DG, AD, and IKA engages lipid catabolism and can revert physiologically pertinent metabolic syndromes.

TFEB is required for the lifespan extension induced by starvation/calorie restriction and autophagy in vivo⁸. Thus, it was investigated if chemically activated TFEB was sufficient to modulate lifespan. The nematode C. elegans was selected as a relevant animal model, as the C. elegans TFEB ortholog HLH-30 engages the CLEAR motif to induce the expression of orthologous TFEB targets and autophagy in vivo^{7,8}. A sterile fem-1 (hc17ts) and dal-1 (dt2300) background was chosen to facilitate the lifespan assay and accumulation of xenobiotics⁴⁹, respectively. fem-1(−) animals have temperature-sensitive fertility defects, which simplifies lifespan analysis as mothers do not need to be continuously separated from progeny. dal-1(−) worms are healthy but have permissive oral chemical bioavailability via enhanced intestinal absorption. Among the 3 compounds, IKA-treatment was found to induce nuclear accumulation of GFP-tagged HLH30 within intestinal cells in adults (FIG. 6J). IKA significantly extended the lifespan of the fem-1(−);dal-1(−) animals (FIG. 6K and Supplementary Table 2).

SUPPLEMENTARY TABLE 1
Characterization of AD- and IKA-loaded
PEG₅₀₀₀-PLA₅₀₀₀ nanoparticles.

| | PEG-PLA | PEG-PLA + AD | PEG-PLA + IKA |
|---|---|---|---|
| D [symbol] ( [symbol] m)^{a} | 67.9 | 68.1 | 68.2 |
| PDI^{b} | 0.168 | 0.172 | 0.15 |
| Loading content (% wt) | N/A | 5.4 | 3.5 |

^{a,b}The hydrodynamic diameter (D [symbol] ) and polydispersity index (PDI) were analyzed by dynamic light scattering analysis. [symbol]
indicates data missing or illegible when filed

SUPPLEMENTARY TABLE 2
Statistical analysis of the lifespan experiments.

| | | Mean Lifespan ± | | Observed/ | Lifespan | |
|---|---|---|---|---|---|---|
| Compound | | s.e.m (Median | 75th % | Total | increase | |
| treatment | | lifespan in days) | (Day) | events | (%) | p-vlaues |
| 1st | DMSO | 18.5 ± 0.5 (18) | 22 | 98/100 | N/A | N/A |
| | IKA | 21.8 ± 0.6 (22) | 16 | 97/100 | 22.2 | >0.001 |
| 2nd | DMSO | 18.8 ± 0.5 (17) | 22.5 | 80/80 | N/A | N/A |
| | IKA | 23.0 ± 0.5 (24) | [symbol] | 79/80 | 22.3 | <0.001 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
WO 2013/152059
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22(3):659-661, 2008.
Smith, March's Advanced Organic Chemistry: Reactions, Mechanisms, and Structure, 7^{th} Ed., Wiley, 2013.
1. Yang, L., Li, P., Fu, S., Calay, E. S. & Hotamisligil, G. S. Defective hepatic autophagy in obesity promotes ER stress and causes insulin resistance. Cell Metab. 11, 467-478 (2010).
2. Singh, R. et al. Autophagy regulates lipid metabolism. Nature 458, 1131-1135 (2009).
3. Ding, W. X. et al. Autophagy reduces acute ethanol-induced hepatotoxicity and steatosis in mice. Gastroenterology 139, 1740-1752 (2010).
4. Hansen, M. et al. A role for autophagy in the extension of lifespan by dietary restriction in C. elegans. PLoS Genet. 4, e24 (2008).
5. Pyo, J.-O. et al. Overexpression of Atg5 in mice activates autophagy and extends lifespan. Nature communications 4 (2013).
6. Rubinsztein, D. C., Mariño, G. & Kroemer, G. Autophagy and aging. Cell 146, 682-695 (2011).
7. Settembre, C. et al. TFEB controls cellular lipid metabolism through a starvation-induced autoregulatory loop. Nat. Cell Biol. 15, 647-658 (2013).
8. Lapierre, L. R. et al. The TFEB orthologue HLH-30 regulates autophagy and modulates longevity in Caenorhabditis elegans. Nat. Comm. 4 (2013).
9. Sardiello, M. et al. A gene network regulating lysosomal biogenesis and function. Science 325, 473-477 (2009).
10. Settembre, C. et al. TFEB links autophagy to lysosomal biogenesis. Science 332, 1429-1433 (2011).
11. Settembre, C. et al. A lysosome-to-nucleus signalling mechanism senses and regulates the lysosome via mTOR and TFEB. The EMBO journal 31, 1095-1108 (2012).
12. Medina, D. L. et al. Lysosomal calcium signalling regulates autophagy through calcineurin and TFEB. Nat. Cell Biol. 17, 288-299 (2015).
13. Martina, J. A., Chen, Y., Gucek, M. & Puertollano, R. MTORC1 functions as a transcriptional regulator of autophagy by preventing nuclear transport of TFEB. Autophagy 8, 903-914 (2012).
14. Carr, C. S. & Sharp, P. A. A helix-loop-helix protein related to the immunoglobulin E box-binding proteins. Mol. Cell. Biol. 10, 4384-4388 (1990).
15. Hodgkinson, C. A. et al. Mutations at the mouse microphthalmia locus are associated with defects in a gene encoding a novel basic-helix-loop-helix-zipper protein. Cell 74, 395-404 (1993).
16. Zhao, G.-Q., Zhao, Q., Zhou, X., Mattei, M. & De Crombrugghe, B. TFEC, a basic helix-loop-helix protein, forms heterodimers with TFE3 and inhibits TFE3-dependent transcription activation. Mol. Cell. Biol. 13, 4505-4512 (1993).
17. Roczniak-Ferguson, A. et al. The transcription factor TFEB links mTORC1 signaling to transcriptional control of lysosome homeostasis. Science signaling 5, ra42 (2012).
18. Martina, J. A. et al. The Nutrient-Responsive Transcription Factor TFE3, Promotes Autophagy, Lysosomal Biogenesis, and Clearance of Cellular Debris. Science signaling 7, ra9 (2014).
19. Martina, J. A., Diab, H. I., Brady, O. A. & Puertollano, R. TFEB and TFE3 are novel components of the integrated stress response. The EMBO journal 35, 479-495 (2016).
20. Pastore, N. et al. TFE3 regulates whole-body energy metabolism in cooperation with TFEB. EMBO Molecular Medicine, e201607204 (2017).
21. Li, Y. et al. Molecular basis of cooperativity in pH-triggered supramolecular self-assembly. Nature communications 7 (2016).
22. Wang, C. et al. A nanobuffer reporter library for fine-scale imaging and perturbation of endocytic organelles. Nature communications 6 (2015).
23. Ma, X. et al. Ultra-pH-sensitive nanoprobe library with broad pH tunability and fluorescence emissions. J Am. Chem. Soc. 136, 11085-11092 (2014).
24. Kabeya, Y. et al. LC3, a mammalian homologue of yeast Apg8p, is localized in autophagosome membranes after processing. The EMBO journal 19, 5720-5728 (2000).
25. Jomon, K., Kuroda, Y., AJISAKA, M. & SAKAI, H. A new antibiotic, ikarugamycin. The Journal of antibiotics 25, 271-280 (1972).
26. Pankiv, S. et al. p62/SQSTM1 binds directly to Atg8/LC3 to facilitate degradation of ubiquitinated protein aggregates by autophagy. J. Biol. Chem. 282, 24131-24145 (2007).
27. Hundeshagen, P., Hamacher-Brady, A., Eils, R. & Brady, N. R. Concurrent detection of autolysosome formation and lysosomal degradation by flow cytometry in a high-content screen for inducers of autophagy. BMC Biol. 9, 38 (2011).
28. Glynn, I. The action of cardiac glycosides on sodium and potassium movements in human red cells. The Journal of physiology 136, 148 (1957).
29. Doughty-Shenton, D. et al. Pharmacological targeting of the mitochondrial phosphatase PTPMT1. J Pharmacol. Exp. Ther. 333, 584-592 (2010).
30. Mukai, H. et al. FKBP12-FK506 Complex Inhibits Phosphatase Activity of Two Mammalian Isoforms of Calcineurin Irrespective of Their Substrates or Activation Mechanisms1. J Biochem. (Tokyo) 113, 292-298 (1993).
31. Kissinger, C. R. et al. Crystal structures of human calcineurin and the human FKBP12-FK506-calcineurin complex. Nature (1995).
32. Hogan, P. G., Chen, L., Nardone, J. & Rao, A. Transcriptional regulation by calcium, calcineurin, and NFAT. Genes Dev. 17, 2205-2232 (2003).
33. Hoyer-Hansen, M. et al. Control of macroautophagy by calcium, calmodulin-dependent kinase kinase-β, and Bcl-2. Mol. Cell 25, 193-205 (2007).
34. Sullivan, J. E. et al. Inhibition of lipolysis and lipogenesis in isolated rat adipocytes with AICAR, a cell-permeable activator of AMP-activated protein kinase. FEBS Lett. 353, 33-36 (1994).
35. Zhou, G. et al. Role of AMP-activated protein kinase in mechanism of metformin action. J. Clin. Invest. 108, 1167 (2001).
36. Tokumitsu, H. et al. STO-609, a specific inhibitor of the Ca2+/calmodulin-dependent protein kinase kinase. J Biol. Chem. 277, 15813-15818 (2002).
37. Clapham, D. E. Calcium signaling. Cell 131, 1047-1058 (2007).
38. Berg, T., Stromhaug, P., Lovdal, T., Seglen, P. & Berg, T. Use of glycyl-L-phenylalanine 2-naphthylamide, a lysosome-disrupting cathepsin C substrate, to distinguish between lysosomes and prelysosomal endocytic vacuoles. Biochem. J. 300, 229-236 (1994).
39. Lytton, J., Westlin, M. & Hanley, M. R. Thapsigargin inhibits the sarcoplasmic or endoplasmic reticulum Ca-ATPase family of calcium pumps. J Biol. Chem. 266, 17067-17071 (1991).
40. Zhang, X. et al. MCOLN1 is a ROS sensor in lysosomes that regulates autophagy. Nature communications 7 (2016). 41. Yuan, Z. et al. Na/K-ATPase tethers phospholipase C and IP3 receptor into a calcium-regulatory complex. Mol. Biol. Cell 16, 4034-4045 (2005).
42. Finck, B. N. & Kelly, D. P. PGC-1 coactivators: inducible regulators of energy metabolism in health and disease. J. Clin. Invest. 116, 615-622 (2006).
43. Zhang, Y.-N., Poon, W., Tavares, A. J., McGilvray, I. D. & Chan, W. C. Nanoparticle-liver interactions: Cellular uptake and hepatobiliary elimination. J Controlled Release (2016).
44. Klibanov, A. L., Maruyama, K., Torchilin, V. P. & Huang, L. Amphipathic polyethyleneglycols effectively prolong the circulation time of liposomes. FEBS Lett. 268, 235-237 (1990).
45. Kharitonenkov, A. et al. FGF-21 as a novel metabolic regulator. J Clin. Invest. 115, 1627-1635 (2005).
46. Markan, K. R. et al. Circulating FGF21 is liver derived and enhances glucose uptake during refeeding and overfeeding. Diabetes 63, 4057-4063 (2014).
47. Lundasen, T. et al. PPARu is a key regulator of hepatic FGF21. Biochem. Biophys. Res. Commun. 360, 437-440 (2007).
48. Renquist, B. J. et al. Melanocortin-3 receptor regulates the normal fasting response. Proc. Natl. Acad. Sci. U.S.A 109, E1489-E1498 (2012).
49. Paulson, C. C. (SOUTHERN METHODIST UNIVERSITY, 2009).
50. Miyakawa-Naito, A. et al. Cell signaling microdomain with Na, K-ATPase and inositol 1, 4, 5-trisphosphate receptor generates calcium oscillations. J Biol. Chem. 278, 50355-50361 (2003).
51. Maron, D. J., Fazio, S. & Linton, M. F. Current perspectives on statins. Circulation 101, 207-213 (2000).
52. Thomas, C., Pellicciari, R., Pruzanski, M., Auwerx, J. & Schoonjans, K. Targeting bile-acid signalling for metabolic diseases. Nat. Rev. Drug Discov. 7, 678-693 (2008).
53. Sudhop, T. et al. Inhibition of intestinal cholesterol absorption by ezetimibe in humans. Circulation 106, 1943-1948 (2002).
54. Chang, G.-R. et al. Rapamycin protects against high fat diet-induced obesity in C57BL/6J mice. J. Pharmacol. Sci. 109, 496-503 (2009).
55. Wilkinson, J. E. et al. Rapamycin slows aging in mice. Aging Cell 11, 675-682 (2012).
56. Cabreiro, F. et al. Metformin retards aging in C. elegans by altering microbial folate and methionine metabolism. Cell 153, 228-239 (2013).
57. Giugliano, D. et al. Metformin improves glucose, lipid metabolism, and reduces blood pressure in hypertensive, obese women. Diabetes Care 16, 1387-1390 (1993).

1. A method of screening for an agonist of a basic helix-loop-helix leucine zipper transcriptional factor of the microphthalmia-associated transcription factor (MITF)/transcriptional factor E (TFE) family (MiT), comprising:
a) incubating a cell expressing a fluorescent-labeled autophagy-related polypeptide with a UPS nanoparticle solution for a first time period sufficient for an autophagy-associated organelle within the cell to uptake the UPS nanoparticle;
b) contacting the UPS nanoparticle-treated cell with a molecule for a second time period sufficient for the cell to uptake the molecule;
c) measuring a fluorescence signal of the fluorescent-labeled autophagy-related polypeptide; and
d) comparing the fluorescence signal with a control, wherein a decrease in fluorescence signal indicates the molecule is an agonist against a basic helix-loop-helix leucine zipper transcriptional factor of the MITF/TFE family.

2. The method of claim 1, wherein the basic helix-loop-helix leucine zipper transcriptional factor of the MITF/TFE family is transcription factor EB (TFEB), transcription factor E3 (TFE3), transcription factor EC (TFEC), or microphthalmia-associated transcription factor (MITF).

3. The method of claim 1, wherein the basic helix-loop-helix leucine zipper transcriptional factor of the MITF/TFE family is transcription factor EB (TFEB).

4. The method of claim 1, wherein the basic helix-loop-helix leucine zipper transcriptional factor of the MITF/TFE family is transcription factor E3 (TFE3).

5. The method of claim 1, wherein the UPS nanoparticle solution has a buffering capacity of between about pH 4.4 and about pH 4.7.

6. The method of claim 1, wherein the UPS nanoparticle solution has a buffering capacity of about pH 4.7.

7. The method of claim 1, wherein the UPS nanoparticle solution has a buffering capacity of about pH 4.4.

8. The method of claim 1, wherein the autophagy-associated organelle comprises autophagosome, amphisome, phagophore, endosome, or lysosome.

9. The method of claim 1, wherein the autophagy-associated organelle comprises autophagosome.

10. The method of claim 1, wherein the UPS nanoparticle solution inhibits the formation of autolysosome by the autophagosome and/or amphisome.

11. The method of claim 1, wherein the molecule overrides the inhibitory activity of the UPS nanoparticle by inducing activation of TFEB and/or TFE3.

12. The method of claim 1, wherein the molecule is a small molecule compound.

13. The method of claim 1, wherein the molecule is a protein or a peptide.

14. The method of claim 1, wherein the molecule is a peptidomimetic.

15. The method of claim 1, wherein the molecule is a polynucleotide.

16. The method of claim 1, wherein the fluorescent-labeled autophagy-related polypeptide comprises LC3, p62, NBR1, or NDP52.

17. The method of claim 1, wherein the fluorescent-labeled autophagy-related polypeptide comprises a fluorescent moiety.

18. The method of claim 17, where the fluorescent moiety comprises a fluorescent molecule or a fluorescent protein.

19. The method of claim 1, wherein the fluorescent-labeled autophagy-related polypeptide comprises a fluorescent protein.

20. The method of claim 19, wherein the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyanl, Midori-Ishi Cyan, TagCFP, mTFP1, enhanced yellow fluorescent protein (EYFP), Topaz, MCitrine, YPet, TagYFP, PhiYFP, ZsYellow1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRed1, mRaspberry, dKeima-Tandem, mPlum, or AQ143.

21. The method of claim 1, wherein the autophagy-related polypeptide is exogenously labeled with a fluorescent moiety.

22. The method of claim 1, wherein the autophagy-related polypeptide is labeled with a fluorescent protein.

23. The method of claim 1, wherein the autophagy-related polypeptide is a fusion protein comprising a fluorescent protein.

24. The method of claim 1, wherein the autophagy-related polypeptide is a LC3 polypeptide.

25. The method of claim 24, wherein the LC3 polypeptide is labeled with a fluorescent moiety.

26. The method of claim 24, wherein the LC3 polypeptide is labeled with a fluorescent protein.

27. The method of claim 24, wherein the fluorescent-labeled LC3 polypeptide is a GFP-LC3 fusion polypeptide.

28. The method of claim 24, wherein the GFP-LC3 fusion polypeptide comprises a LC3-II polypeptide.

29. The method of claim 24, wherein the GFP-LC3 fusion polypeptide comprises about 80%, 85%, 90%, 95%, 96%, 97%, 98%, or 99% sequence identity to a LC3 sequence as set forth in NCBI Accession number: NP_115903.1.

30. The method of claim 1, wherein the first time period is between about 1 hour and about 36 hours, about 2 hours and about 32 hours, about 5 hours and about 24 hours, about 8 hours and about 18 hours, about 10 hours and about 15 hours, about 8 hours and about 24 hours, or about 12 hours and about 18 hours.

31. The method of claim 1, wherein the first time period is at least 1 hour, 2 hours, 3 hours, 5 hours, 6 hours, 7 hours, 8 hours, 9 hours, hours, 12 hours, 18 hours, 24 hours, 36 hours, or more.

32. The method of claim 1, wherein the second time period is at least 30 minutes, 1 hour, 2 hours, 3 hours, 4 hours, 5 hours, 6 hours, or more.

33. The method of claim 1, wherein the control is an equivalent cell comprising a fluorescent-labeled autophagy-related polypeptide incubated with a UPS nanoparticle solution in the absence of the molecule.

34. The method of claim 1, wherein the cell is from a human.

35. The method of claim 1, wherein the molecule is identified as an agonist if the molecule promotes nuclear localization of TFEB and/or TFE3.

36. The method of claim 1, wherein the molecule is identified as an agonist if the molecule promotes dephosphorylation of TFEB and/or TFE3, optionally through the calcium/calmodulin-dependent dephosphorylation by calcineurin protein phosphatase.

37. The method of claim 1, wherein the molecule is identified as an agonist if the molecule inhibits mTORC1 or the mTORC1 pathway.

38. The method of claim 1, wherein the molecule is identified as an agonist if the molecule inhibits the 5′-adenosine monophosphate-activated protein kinase (AMPK)-mammalian target of rapamycin (mTOR) pathway.

39. The method of claim 1, wherein the molecule is identified as an agonist if the molecule induces lysosomal, mitochondrial and/or endoplasmic reticuli (ER)-specific release of Ca²⁺.

40. The method of claim 1, wherein the molecule is identified as an agonist if the molecule is an agonist of calcineurin protein phosphatase.

41. The method of claim 1, wherein the molecule is identified as an agonist if the molecule directly or indirectly activates TFEB.

42. The method of claim 1, wherein the molecule is identified as an agonist if the molecule directly or indirectly activates TFE3.

43. A method of screening for an agonist of a basic helix-loop-helix leucine zipper transcriptional factor of the microphthalmia-associated transcription factor (MITF)/transcriptional factor E (TFE) family (MiT), comprising:
a) incubating a cell expressing a fluorescent-labeled LC3 polypeptide with a UPS nanoparticle solution for a first time period sufficient for an autophagosome within the cell to uptake the UPS nanoparticle;
b) contacting the UPS nanoparticle-treated cell with a molecule for a second time period sufficient for the cell to uptake the molecule;
c) measuring a fluorescence signal of the fluorescent-labeled LC3 polypeptide; and
d) comparing the fluorescence signal with a control, wherein a decrease in fluorescence signal indicates the molecule has an agonist activity against a basic helix-loop-helix leucine zipper transcriptional factor of the MITF/TFE family.

44. A method of screening for a transcription factor EB (TFEB) agonist, comprising:
a) incubating a cell expressing a fluorescent-labeled autophagy-related polypeptide with a UPS nanoparticle solution for a first time period sufficient for an autophagy-associated organelle within the cell to uptake the UPS nanoparticle;
b) contacting the UPS nanoparticle-treated cell with a molecule for a second time period sufficient for the cell to uptake the molecule;
c) measuring a fluorescence signal of the fluorescent-labeled autophagy-related polypeptide; and
d) comparing the fluorescence signal with a control, wherein a decrease in fluorescence signal indicates the molecule is a transcription factor EB (TFEB) agonist.

45. A method of screening for a transcription factor E3 (TFE3) agonist, comprising:
a) incubating a cell expressing a fluorescent-labeled autophagy-related polypeptide with a UPS nanoparticle solution for a first time period sufficient for an autophagy-associated organelle within the cell to uptake the UPS nanoparticle;
b) contacting the UPS nanoparticle-treated cell with a molecule for a second time period sufficient for the cell to uptake the molecule;
c) measuring a fluorescence signal of the fluorescent-labeled autophagy-related polypeptide; and
d) comparing the fluorescence signal with a control, wherein a decrease in fluorescence signal indicates the molecule is a transcription factor E3 (TFE3) agonist.

46-151. (canceled)

152. A cell composition comprising:
an engineered cell expressing a fluorescent-labeled autophagy-related polypeptide;
a UPS nanoparticle solution that buffers an autophagy-associated organelle within the engineered cell to a pH range of between about pH 4.4 and pH 4.7; and
a molecule incubated with the engineered cell, wherein the molecule is incubated with the engineered cell to determine whether it is an agonist against a basic helix-loop-helix leucine zipper transcriptional factor of the microphthalmia-associated transcription factor (MITF)/transcriptional factor E (TFE) family (MiT) expressed in the engineered cell.

153-181. (canceled)

182. A cell composition comprising:
an engineered cell expressing a fluorescent-labeled LC3 polypeptide;
a UPS nanoparticle solution that buffers an autophagosome within the engineered cell to a pH range of between about pH 4.4 and pH 4.7; and
a molecule incubated with the engineered cell, wherein the molecule is incubated with the engineered cell to determine whether it is capable of an agonist activity against a basic helix-loop-helix leucine zipper transcriptional factor of the microphthalmia-associated transcription factor (MITF)/transcriptional factor E (TFE) family (MiT) expressed in the engineered cell.

183-195. (canceled)

196. A method of treating a metabolic disease or indication, diabetes or a diabetes-related disease, metabolic-related obesity, non-alcoholic fatty liver disease, non-alcoholic steatohepatitis, or an aging-related disease or disorder in a subject in need thereof comprising administering a therapeutically effective amount of a molecule identified by the method of claim 1.

197-201. (canceled)

202. A method of modulating an immune response due to a pathogenic infection in a subject in need thereof comprising administering a therapeutically effective amount of a molecule identified by the method of claim 1.

203-208. (canceled)

209. A composition comprising a molecule identified by the method of claim 1 and a block copolymer capable of forming a micelle.

210-231. (canceled)
